SEQUENCE LISTING (17 sequences)

SEQ ID NO: 1 - DNA, 32767 bp, unidentified
  source 1..32767: mol_type = other DNA; note = Acutalibacter nov. genus
gcgccgtctgatttttgctcgtcggctcgtggtctaaaatgcccgtcttgccaacaaacagcttcgcaagctcctcgagtgtctctttcgtaaagcactcaccgtctctatccacctcgttgtcgcacaaaacgaccgaaaacgtgtacacctcctccggcttataggcgcggcgcgtgtaaaggttgatcttctccatcgccgctttatccggctgtaccgccgttaccgtttcgctcaagcttccccctcctttaaaagctgctccgtcttcgccatgagatacgccgcgttcgcgtgatcgacttcgtcctgcatcgtgatctcgttccaatcgatttccaccgtgtcgtcaagaccatttagccgcagccacgttttgcagatcttcaaaaccgccggctccaaaattctgcggtacgccgtcagctcgctcgtcagcatgtccgcctgctggctcgacatgcgctccgtcgaagaccacgaaagccccagcaaaaacggcggaatgcccattttcgccacaatctgctccagcatctgccgcacgggcacttcgctgtccatctgctgcccgtccgcgccgatggctttaatttgcacgtcgcccaccgccacaaagtcactcacgccggggccgtgcatcgcacggctccactcctttgcaagctgccccgcgcggtcacccgcgttcatgtagccctcgtttttgcacgtcaccgcaaagcgcacattgcccatacgctcccagttgatgccaacggtgcggtaaattttcaaaagcacctcgctcacaaacggcaggccctttaaaatggagcttcccaccgcgctgcccggcatcgggtttaacgccgaagtcaaaatgagctccgggtagcggcacggctccgcacgcccgtcccttacgacgaacacgcccgtttcaagcggcgagatgagccgaatctcgaggtcttcgagcgacgcattatacagcgcccgaatttccccgtttgcgagtacgatctccccaattgcattgccatacgttaaaagctgctcaaaataggccgataaaaagttatcgatgcccatgttgcagccgtttacgggcacggtttttaaaaacccgcgcagcgcgttttccgtgtgcttttcggggcaatacacctcaaacccgccgacaagacgcaccagcttgtaaattgccgcgtcgatgatcggcacctgctcgcgcaaggcacggtacagtgcacgctcgcctgccgtcaccggcagcaccggcgggtacccgccctgcgtctgcaccgaaacggccgtgcgtttctcctttttcaaaaatcccaaatgctaactcctttccccgtctccctttgcaccgcgcaggcaaagctttcgcccgcgccgagcaccgtggagacaaaatagcggatgtcgtccatggcatggtcattctccttcacaggcacatcccgcgcgccgtcgtctgcccagcggtagagggaaaactcccgcaccgcatccacgcagccctttgtcaccttgatctgccccgtgcgcagcgccgttgccgtcatgcggatgccgtttaaaacgtcgtttttcgccgccgtcactgcaaaaccacggctctgcaaaagtgtgataaagctcgccgccgaggggtcgcatacgatgacattgggcgttctgccgcaaatgagctccaaaaagcccttaaagtgctcctcgtccgtgcgccgcgtgttttcaagccgcgcgtcaaagtaatactcgtctgcgcggtaccacacgccgcacaaaagcccccaaaggcccatggaggtcgggtttaccgtgccgtaatcgatggacaccgcgtaccgctcaaaatccgctttcggcacttcgcacaggaggtcaagcgcaaacgggtaaatcagtcctgtcgccgccgtccacttgccctcgatgaaccggctgtaaaacgcgccggaaaacatcgttcggtagcgcttgcgcacgtcttcactcagcgacggattatcctccatcgtaaagtgtaaatacagcgcgtttcggctctcggctttttgtatccactcgcggtaaaaccagtgctgcgggtgctccgggttgcagttgaaccacagcttcgcgttttttagcgagcagcgcgcaatgccctgctccacaaaagagcgcggcatcagcgccacctcgtcaaacaaaacgccgccgagcgtcatgccctgaatgagcgccgcgctcgattcatctcgtccgccgaaaaggtaaaagcggttttctctgccctgatacgataggataatctcgttctctgcctgccgcacacgcatggaaaagcccatagattttagctttggtataagctccgcgaccacatttctccgcacagaacggatcgttttgccgcacaccgcaaagctcgtattttgaaagcgtgccatcgcccacagaaaaaaggatatccccatacaggtggttttgccgctgcgcaccgcgccgtcgcagatgacggcaagcttatcccggtgcgggcttttttcgccccaccagcataagacctgcatctgcttttcggaaaacggcgtcacggcttatccccgtctttcagcgccgccgcaccgctttcaatggcggagagcagcgcctgtgcacccgtcccgctcgtctcgagctcactgaggagccgcagcgcttcgatccggtcgaaaaaccgaatttccatgccgccgtttttcggtctgcgaatttcggagacggcgaacagatccatgtttttcagcgtgcgcgggttcacgtcctcgcaaaagagcagccttacggcatcatgaatgccgccgaacgccaatctgcgaaagccctccttcgccgtttcgttcaccatgttccacatcctctctgaaagttttgaagcgccaaacggcgttcgttttttcgcccgcctgtttgtctcttcacccatacccccaaaacgcagaaaagtttccctcaaaagggaaacttttcaaattcatgtcctccgatgtcctgcaatgtcctacaatgtcctctgatgtccggcgatgtccggcgatgtccggcggtttttattcaatttcaggctccctctgattgaggaaaaggaagagataacgcgcatcaacgcatgctcacagctatataaaaagggcggcatggagccgccctttcagggaacctatgcaactgggtttcctatggctaaacagtcccccggactgttcaagcaatgcaaagcattgctttctaccattcttagttttttgttaggcaataagatttcgctttctgcggaaagcgacttagggcgctgccctaagaacccgcaagttttctgaagaaaacttgaccaaagactttatttgtcgttctccgtgagcacatcgtgcatcaaaaacggcagacatttttcgaatttcacgccccaaatggtgttgtcaccgcacgcttttctcgtttcttctatgcactttgcggtgtaccgcacggcggcggcgcaggcttcctttaccgttttgccgtttaacatcgcccccagcatcacggccgcgaaaatatcccccgtgccgtggtatttgcccggcacgcggtcggaaagcaccagcgccgtctcgtctttttccctatcgtagcacgccgcgccgattttctccccgtccgtatacgcacccgtcaagatgacgaccttcggcccaaagtcggaaagggctttcattactcttttaagctccttttcgtcatacggcccgtccttaaacggctcgtgcaaaaggcaatacgcctctgtcatgttcggcgttaaaatgtccgcttttttgcaaagcctcgtcatgctctgcgccgtttctttattaaaaatgccgtacagcgtgccgttgtcgcccatcacggggtctacaaggcgcagcgggcggctgtgttcgtccgtaaacagctcgaagatttcccgcacagtgtcaatctgctcgcggctgctgaggtatccggagcagatgccctcaaacgcggccccctcgcgcttccagtgctgtgccgttttcagcatatcgtccgtcaaatcgcggtacacgggatctttaaagccgccgtgtgtggaaagcaccgccgtcggcagcacggatgtctcgcaccctaaggcggacagcaccggcagcatcaccgtaagcgagcatttccccgcaccggaaagatcgttgatgaccgccgcgcggcgcggtttttcttctgtatttaaaaactgcatcgggtttttccctcatttcaaaagtatttccgcttttattgtaaccgcggcggaaaataaagtcaattagcggtttacaggtgaaaaaaagaatggtaaaataaagggtacacgtacaggcatgccctgcacccgcgaaaggagctttctatgaaaaccgaaaagcaacgcaagctcatcgttcgcatcggcgcggcgattttggccgggctgatgatcctctcggcactttccgccgtactattcagttgaatacattcaaccgtagaaaaaattaaccccttgaaaatacattgtaaaggaatgatccacccatgagtaaaccgaagtattatatttcgacggctatcgcctacacctccagccgcccgcacatcggcaacacgtatgagatcgtgctcgccgacagcattgcgcgctataagcgcatggagggctacgatgtctacttccagaccggcacggacgagcacggccagaagattgaagaaaaggccaatgccgccggcatcaccccgaaggaatatgtcgacaacgttgcgaagattgtcaaggcgaactgggacgtgatgaacacctcctacgataaattcatccgcaccacggacgattaccacgaaaagcaggtacaaaagatctttaaaaagctctacgagcagggcgatatttataagggccactacgagggcatgtactgcacgccgtgtgagtccttcttcaccgagtcccagcttgtcgacggcaagtgcccggactgcggcggcgaggtcaagcccgcgaaggaagaggcatacttcttcaagatgagcaaatatgcagaccgcctcatcgaccacatcaataagcacccggagttcattcagccgccgcagcgcaaaacggaaatgatgaacaacttccttctgcccggcctgcaggatctctgcgtttccagaacctccttcaagtggggcattccggtcgattttgacgacaagcacgtcgtgtatgtttggctcgacgcgctcacaaactacatcaccggcatcggttacgactgcgacggcaactccaccgagcagttcaagcgtgattggccggcagacctgcacctcatcggcaaggacatcatccgtttccacaccatctactggccgattttcctcatggcgctcgacctgccgctgccgaagcaggtgttcggtcacccgtggctcctgatgaacggcggcaaaatgagcaaatcgaagggcaacgtcatttacgcagaagaccttgtagacctcttcggcgtagatgcggtgcgctatttcgtcttgcacgagatgccgtttgaatccgacggcaccatcacgtgggatctgatgatcgaacgcacgaactccgaccttgcaaacacgctcggtaaccttgtaaaccgtaccatttccatgacaaacaagtacttcggcggcgttgtagagaataaaaacgtcgtagaagatgtcgataacgatttaaagagcgttgtgctcgccacaaagaacagcgtgcagacgaagatggatcagctgcgcgtggcagatgccatttccgaaatcttcacgctctttaagcgctgcaataagtacattgacgaaacgatgccgtgggcgcttgcaaaggacgaagcgaaagccgacagactttccaccgttctctataatcttgtggaaagcatcacgatcggcgcggcgctcctcacaccgtttatgccggacaccgcagagaagatcgtcgcacagcttggcacgaccttgcgcgcatacgatacgctcgatacgttcggcgtttacccgaatgggggtaaagtaacagacaagccggagattctcttcgcccgtatcgatccgaaggagatggacgagaagattgccgctgtggaagcgaagtacgctccgaaggaagagccgaagaaggaaattgagggtcttgcccagatcaatattgaagactttgcaaaggttgagctgcgcgccgcaaaggtcgttgcctgcgagccgatcaagcgtgcaaagaagctcttgaagctcacgctcgacgacggcacctccacaccccgcacggtcgcgtccggcatcgcgaagtggtataagccggaagacctcatcggtcacaccgtcatcgtcgtcgcgaacctgaagcccgccgtactctgcggcatggaaagccagggcatgatccttgcggcagacgcaggcgaggacgatgtgcgcgtcgtatttgtggacggcaccccggcaggctccaaaattcgctaaggaaaatccctatgatatttgattcccacgcacattacgacagcgagcagttcgacatcgaccgtgacgagctgctcggctccgtactgccgcaaaagggcgtcgttgcggtcatcaatatggcagccgattatgaaagcctcaaaaaaaccgtggcgctctgtgaaaaatacgattatatctacggtgcagtcggcatccacccggagtgcgcaaacgacctgcccgataactggctttcggacgtggaagcgctgctttcccacccgaagatcgtcgccgtcggcgagatcggcctcgactaccactgggaggacgagtgcccgcgcgaaaagcagaaagaggtgcttatcgcccagctggagctcgcgaaaaaacacgacctgcccgttgtcatccacgaccgcgaggcccacggcgatatgatggacattctgcggaaatacaagccgaagggcgtcgtgcactgcttttccggcagtgcggaattagcacaggagaccgtaaagctcggcatgtacatcggccttggcggtgttgtgacgtttaaaaacgcgcgccacagcgtggaggtggcaaagaccatcccgctcgaccgccttttgaccgaaacggacgcgccctacatggcgcctgtgccattccgcggcaagcgctgcgacagctccatgatcccctacgacgcggcaaagatcgccgaagtgcgcggcatcacggcgcaggaggttttggaaaccgcctgcgaaaacgcctgccgccttttcggtatcacgctgccgagcgccgcaaaataaaaggagcgatcatcttgaccatcacgtatgaatacggcgataacctctacatcaacgtcacgaaccagtgcccgaacgcatgcgacttctgcctgcgcaacaacagcacgggcagcctgtacgcagataacctctggtacgagggcgcggagccgacaaaagaggaaatcttagccgaccttaaaaaacgcgacctcaattcatacgcggagattgtcttctgcggctacggcgagcctgcctgccgttgggacgacatgctttggcttgccgcgcgcttaaaagaaatgggcaattacaccctgcgcgtcaacacaaacggccttgcagactgcatcaccgggcgcaacgccgcgccggaaatgcacgggcttttggacattgtgtccgtcagcttaaacgcgccgaccgctgaaaagtacgatgcggtatgtcactcgaaatacggccccgaggcgtttgatgccatgctgcattttacggcagaagccgtaaaaaacgtgccgcatgtggtgatgacggtcgtcagcaccatgcccgcagaggacattgaagcctgccgcaagctgtgcgaaaacaccggcgccaagttccgcgtacgcgagtatatctccgaataactgcataaaataacgaaagcagagtgctcaatccgaacactctgctttttgttttgtgattttagtttaatttaatcttgaatgtgaccggcttaccggacgtgtagttcgtcttgatatgcagcccattttcgtcacgcgtccacgcgggtgcatcgtctgtgccgagcaccgaaacgctctcgactaaaccgtggtacaccgtaccggcggtgtgctcctgcatacgcagggagtgaatgtggtattcgccgttgtcgttcgccttcagcgcgatggcaaagagcgtatctccctttgccgtaaagcggaaatcctcgcccgtaaagttctttttcacgccgtcggaaaagccgccgtcctgcacctttgtggggccttcgccgaacgtcctccacacgtgcgtgtcatagattgcttcgccgtttacctgcatccagtcgccgatggcagtcagcactgcggtgtcctcatcggagatggtgccgtccgctttcgggcctacgtttaaaagcagcgcaccgtttttgctgacgatgtccacaaggtcgcagatgatgtcccccgccgggcggaagtcgttgttttccgtgtagcaccaggaattcagcgcaatggcggtatcggtctgccagaaatacggcttgatgtccgccatttgcccgcgctcaatatccggcaccgccgtgccgaacatgtacgcatcgaatttataatcgatggcaacctcctcgccccattccgcagcgcgattatagtagtacgccgccgcttttttgaggtacggttttgccgcctcctgctgaatccaccagtcaaaatagagaattttcgggtgataattgtcgatgagttcgcacgtgcgtacaagccagtcttccatgaattcttcagagggcttgccgtcgatggcatcgaaattctccgggtcgggcatggagggccaatagaggtcgtcgcggtccgggttttgcggcatatcgctctcaaactttttgccgttgctcatgaagaaccagtgctcaatgcggtgcgaggataccccaagcgtcatgccctgcgcttccacggccgctttcagctcgccggctatatcgcgctttgggcccatctcataggcgttccaatgtgaaatatttgagcggtacatctggaaaccgtcatggtgctccgccaccggtacaacgtattttgcgcccgcctttttaaaaagcgcagcccatgcgttcgggtcgaatttctcggccttaaacatcggaatgaaatccttgtagccgaaatccttgtgtgcaccataggttttgatatggtgctcgtagaccttggagccctctatgtacatgttgcgcgggtaccattcgctgtcaaacgccggcacgctgtacacgccccagtggatgaaaatgccgaacttcgccttttgataccacttcggcgtctgccagcttgaaagggacgcccacgtgtctttgtacggtcccttttcgatcacgtcgtcaatgctctgcaatgcttttttcatatcgatcatactattttcctcccaaaaggaacttttctctaagagatttttctctttcccattataccagcacatcagaaatttgcaagacaaaagccgacggaaacccgccggccttttgctgttatttgtttctgtaatagacatcgtttaattctttgagccactttttgtcgatgccgtcgagttgctcctgcgtgacgcggaacgcccagttgccgttcggcacgccgggcacgttcatttttgtgtccttgccgaagccgcagaggtcctgcaccggcacaatgaccgtattcgcggggctctgccacagcgtgcggatgacggcgtggcaagaagcactttcgctgccgccgaccgcccagtcaccgccggggaacgcgcagtatttcagcgcatgctcgcgctcatacggctgtgcctcccacagccagccgagcacggtgttgttatcgtgcgtgccggtataggccacgcagtttttcgggtagttgtgcggcatgtgcatgttgtctttgccttccataaacgcaaactgcatcacgcgcatgcacggcagctgcgttttttccagcaggtcgatcacaccgtcgtcgatgatgccgaggtcctcggcaatgatctttggatttttgaacgtgcggttcacggcgtcgaagaaatccgtcttcgggccgtcgagccactgcccttctttggcggtctccgcttcggcgggcacggcccagtaggcggaaaaagcgcggaagtggtcgatgcgcacggcgtcaaacagcttaaagcacgcgccgaggcgatccatccaccaacggtagccgtcttttttcatctctgcccagttataaagcgggttgccccacttctggccgaactcgctgaaataatccggcggcacgcccgccacattcttcggctcgccgtcctctgtcagatcgaacagatggcggttcgcccacacgtcggcactgtttctcgcaacgtacatcggcagatcgccgatgatctccacccctttgctgtttgcatattttttgatcttcttccactgctcgaagaacgtgtactggatgaactccatgaaaagcacggtctcggcctgctcctccatcgctttcgcgacggcttcggggcgatgaagcttcaggtcttcgtcagcccagtcataccactctgcgccgatattcgcttctttcaagatgatatacagcgcgtagtccggcagccagctcttgttttcctcggcgaacgcgcgtaccttatcgcggtaggcttcgtccgcacgggtaaatgcgcggtataagaccggaatgcggcgcagctccagaaaatcgaacgcggcggaatacgggtttgcatacatctgctcgcgcagctcgtcgtctgtcagtaagccctcgtcgcgcaattgctcgaggtcgatgaaattgcggttgcccgcaaacgccgaaatgctggcatacggcgaattgtacgcgtcggtcggcgtgaacggcaacacctgccaataggtgcagccggtgtcactcaaaaagtcgatgaactctttcgcttccttaccaaagcagccgatgccgtaggcaccgggcagggaggaaacgtgcatcagcacaccgcttgctcttttattcatggacttctcccttcttttgcataaattgctcaaatattgagacattatatctcataaacttgtataagtataccatactttataaggctattcaatggtaatcttacacagatttcagggagaacttttgaattgtacgtcattcgatcaggagcaccacggcgcttacgtcgtcctcgtgtccgtctgtgcggtcttttacggcgcgctccacaatgcgctcggaaagcgcctgcgggtcgtcgtcctcatagtttgcaatgaggtcgcatagccactccgtgccggaagccgtcacgccgtccgagaccatgacgagcatatcgcccgcatcgaggtctttttcgctgcgggcaaaggcgatttccggcagaatgcccacgggcacgcttttggcttccagatactccgtcttcttacgcctgcgcagcaccgtgcccgccgcgcccgccttgcgcagggagacgtgccctgtaaagaggtcgatgcacgcaacatccagcgttgcaagcgactcctccccgccctttgcgagcatggcggagttgatcatcttcagtgtgctctcaaatccgataccggctttgagaagcgactgcgccatacccgcggtcattgtgccgtccacagctgcccggccgcccgtgcccatgccgtcgcttaaaaccgagatataccgcccgctgccgtccgtgaacgcctcggcgcaatcgccgcagagagacgcgccgtttgctgcgtgctgcgcaaagccgcgctgcacggcataaacgggcttctggcacatttgcagctttaaggtatcgcccgccgccgtcacgctcggctgcgtgaacgtgcgcccgcacgcgccggagatctcgcgtgtcagctgccctttgttgatgtgcaccccgtgtgtgcggcaaatgtttgcttcaatcgtcatgcggccgaatttatcgatgcggcagcagacttcaatcggcgtgagctcgcggtggcgcaagacctcgcgcacctgctccgcactgtcctcatcaaagcgctggtactgctcgaattcctccgccatgtcgccgagcatctgcgccgtggagcggaaatgttcttcgacgatggcgcgcacctgttcggcgcgcatacgggcgctttcttttgccaaaagctcgcggtattcgccgtttacggcgtctctcagctccatggcacggttgcagcgcgtcaaaaacggcacatcaaagtcgctgcgttcgattttgccgcgctcccgcagcggatacgaaagccgcgagaacgttttttgcgtctcgtcgcgatttttgcgccagcacactgcgcattggctgcacccggtgcaaaccttctccgccgcgcggccgtacacatgcggcaggtcagccgccgcggtcgtctccagcttttccgagatgcgatctacggaatccgatgcgctgcgcatggcttccgccgtataccgcagacgcaaaataacgttttttctaagagaatcgcccggcagcctgtccccgtgcgcggtgaataaattggaaagccgtcggctctgcggcaaaagcatgtataaaatggaagccgcggccacctccaccgcgccgtaaaacacctgcgaagtactgttcacctgcaaggatgcgatgccgtttgaaatgacgaacgccaccgccacggcgaatttgccgagtggcgagaacacgccggccatcagcccgccgagaccgtacgcgccggagagatacgaaagccccgccgtggagagcccctgcactgtgcccgccgcaatgcccgccaccgcgccgcccgcaagcccgccaatgcgcgcgcagacgagtaccgtcagcacgatgagcacgcgcccgacggaaatgtgcgagacggtcacgtttgaaaacgagagcagcaaaacgccgagcgagacgataacagccgccacgtcgccgctgtcaaacacggcggtctcgcggtgggtctccaaaagctgcacggtgcggcgcagaaaatacgccgtgcccgccgccaaaaacgactccgcaatgtacatggcgagcacatttgaaagcgagccgtttaaaaacaccatggcgatgcccgtcgaaagcagcggcaaaaacgccgtgaccggtgcgaagaccgggtggtcgcggatggactttaactccgacagcgaccagcggatggcccccacggcgagcaaagccgcgatgtagcgcgcggggatatccacgggcgacgggaacaggtagcccaccgcgccgccgaggatgaccgcccacatggttgaaaacggcgctgccgccgcgcctgccgccgcaaacggcgcgtatttgccgaagacgagcccgcgcgccgttaaaaaacccgcaaggccgcccgccatcagccacaccgccctgcgcacatacggcacggtaagggccgcgcttatgcggtcccgcactcttaaaactgctttttcctccattgtcttttccgccctttcctgcactttgtcttttctgtgctaccattatacgaagccttgcccgcaggaaaacacgaaacaggcaggcgggttttaggattgttttgtgaaaagcacaaggtttctgtcgccgcgcctttcatgcgcattttccttgtcggtttctcttgcttcccacgtggaaatttacagttttccggctgtgccgacttgacaaaccgcaaaaaagggcttagaatagcggtaatatcaaaactgatgattggaaatagtacccgatgcaaaacggcaaagagagcccctttcggtgagacggggtgcgggctgcgcggcgaatggctccatgagggcgggcgggaaaatcgcccgacgtttggccgcgttagaagccaagccgtaactacggctgtaaagcgggcgtttatgcgccaatttgggtggtaccgcaggaatttgacctcttgtcccttagagtttgggacaaggggttttactttttggggtgcatctgaaaagtcaaaaacttgtctattccacaaatacggacggtttctgcgttgaaaatcctcggcagccggaaggcttcctgcggttttcgccttgaacccgcctcgtctttgcgaaatatcctgcgtttcctttgttttcagatacacccgtaaaagcctgaaagtgcaaaatcaaatgaatcatatagaaaggtgacgttacgatgaaatacaaattttcagaccgcgtgctcacgttaaagccctcggcaatccgtgaaatctttaaatacgccgctgacccgagctatgtttcgctttccgccggcaaccctgcgccggaagcgttcccgtctaaacagcttgccgccatttccgcaaagctgatggaagaagagccgattttggcgctccagtacagcacaacggagggctacccgccgcttttacagcatcttaaggcgtacatgaagtcctccaaaaacatcggcaccgaggatgatggcgtgctcgtcacaagcggcgcacagcagatcatggatctgttcacaaaatccatcttaaacgaaggtgaaaccgttatttgtgaagcgccgagcttcatcggctctttaaacgacttccgttcctacaaagcaaagctcgtcggcatcccgatggacacggacggcatgaacatggaagcactggaaaaggcgcttgagaccgaaaagaacgtgaagttcatctatacgattccaaactttcaaaacccctccggcatcacgatgagccttgaaaagcgtcaccgcctgtatgagcttgccaaagcgtacgacgtaatgattctcgaagacaacccctacggcgacctttactatgaaggcgagccgctgccgagcatcaagtcgtttgatacggacggcatcgtcctctacgccggttcgttctctaaggtcatctcccccggtatgcgcgtgggctacgccatcggcccaaagccggttctcgccaaaatgaccgtctgcaagcaggggcaggacgtgcacacgaatatctggtcgcaggttctgtgctaccgcttcatgacggaatatgactttgacgcgcaccttgcggggcttagaaaaatctacacgaaaaagcgcgctttgctccttgacctcatggaaaagcaccttgcgccgtacatcacgtgggacccgttcaacggcggtctgttcgcatggtgccacctgccgaagggcgttgacatgatggaattcgtacagaaagcgcttgaaaagaaggtttgcgttgtgccgggcaccgcatttttgacggatgaaaacgagccgtgcgacgcgttccgcatcaatttctccaccccgaccgatgagcaactcacaaagggcatcacgctgcttggcgagaccatccgcgagatggtggaaaaataaaggatatcagagaggaaaatcaccatggaaaataccgtaaacaccacacccgaaacaaaaaagaagcgcgttttgagcatgattcagccgacgggcatgttcacgctcggcaactacctcggtgcgctgaagaacttcgtcgcgctgcagaacgactatgagtgcgtgtacgcgcttgccgacctgcacgccatcaccgtgcgccagaacccggcggaattccgcaaaaacacgctttccgcctatgcgatgatgctcgcgctcggcatcgacccggaaaagagcattttcttcatccagagtcaggtgccggagcacgcacagcttgcatgggtattgaattgctacacccagttcggcgagctctcccgcatgacgcagtttaaggataaatccgcaaagcacgccgataacgtaaacgcggggctgttcacttacccgagcctgatggcggcggatattctgctttataacgccgattacgtgccgatcggtgctgaccagaagcagcaccttgagctttgccgcaacattgcggagcgctttaacgggctttactccccgacattcgtcgtgccggagggtcttatcccgaagaccggcgcgcgcatcatgtctttgcaggacccgacgaagaagatgagcaaatccgatgaaaacacggcgggcttcatcactatgctcgataccccggcggagatcatgaaaaagttcaaacgtgctgtcacggacagtgaagcccgcgtctactacggcgaaaacaaagccggcatcaataacctgatgggcatttacagctgcattacgggcaaaagctatgacgagattgaaaaggaattcgagggccgcggctacggtgatttcaagaccgccgtgggcgaagccgtcgtcaaggagctcgagcccattcaggcgcgctataacgagctcatcaaggacaaggcataccttgaaaagtgctacagcgaggccgcgcctcgtgcagaggccattgcgcgccgtactttgcaaaaggtcatgaagaaagtggggtatttgccggtcgcacactgagcatacgggaattgacaatttctttctaagccagtataatggacatggaaatcgaaaagccgcagcggtctgtttccgcgcggcttttttcttgagaaaataaacgaccctttttacagaaaggatggatccaatggcaaagcacatggccggcaaaaatcagcgcccgtattctgccgaagacagagcgcgccgcgccgcaaaggctgacccgcgcagcacggcttccgagctgacgccggaaacgctgtacgcaaagcaatcgtcacaaagcgaaaagcgcgtcccctcctccatagaaaatctgcattttaccccgaagccgccgaaaagcggtcccgagggctttgaagaaacacgcgaatatcccgtaccggcagcgccgcaacaggaagcgccccgctcggagcgccccgcgcctgcacccgctccacgaccggagccgtatgaggacgaagacgactacgaagagtacgaagaatatgaggaaggctacgacgagaagccgcgccgaaagcgctcgcccgtcgtgcccatcgtcatcgtgatgatcgttttggccatcggcattttggggtacattgcgtacagcctcggcgcgttcggcaagctcttacccgctgccgccacacctacgccggagccgacaccggaagccgccgcaacggcagagccgacacctacccccaccgcagagccgaccgccacgccggagccgacaccgccgccgatctatgacgacggcacggagggctacatgtcctcgggcattctcatctacaataacaagggctttgaaatgttctacggcagcgacagcatggcgaccgcctacgccgaaacgctgaatgactgtgcaagccagctttccggcacaaaggtgtacagcatggtcatcccgaatcactcggaattcggcgtgccggagcgcgtgcgcaattactatgaggaagcttcccagcgtgagaacaccatggcggtcaacaatacgctttccgcaagcatcaccgcggtggatatttacgatgcgctgaacctgcacaacaacgaagacatttattttgataccgatacccactgggcaccgctcggcgcgttttacgcgtataaaaagttctgcgaagcggcaggtgtaacgcccacgacgctcgacagcttcacgaaaacgtcctctgacttcacgggctaccttgcctacgttacgagcgaagacgtgctgaacaacaaccccgatacactcgatctctacgacccgaagtacaactacacatgcgaaatttcgtatgacgggcagtattttgaggaaaccgacagcctcaatagccacgacgagagcctcggctacgccatgtacctgcacggcgatatgggctgcgtgcgcatcacgaaccacgacctttccacgggccgcaagctgcttgtggtgaaggactcctacggcaacgccatgggtccgttcctcggcgcaagctttgacgaggtgcacgtggtggacttccgctactttgaaggcgacctgccgacctactgcacggagcacggcatcacggacgtgcttttcgccgtcaacgaaatggccgtcaataccgagcagcaccagaattcaatccgcgctctgtttgattgaatcagcgcctccgcaccccgaaaaatcgcataaaaatcgcaccgcacgctcaaaaacgagtgtgcggtgcttttttgtttgaagcgctccgttaaatccaaaattcaccgcaagagctccgggtttttttccagaaacgcctgcaagctgccgtcttcgtagcggtaactcatcacaacgccgccggtctgcgtcttttcgcgcgcttgtcccattgcggcgccgaagtgatttacccataaacccacaaaacgcaaagcctgtacacatgcgcgcatagaaaaacggcaccgttacaaaaacgcggtgccgtttgcttttattgggttagatcgacttatattcgtccaaaaggctttgggcgagcttggagttgacgacggcgaaatcgtccggggatttgtaatcgagcgtgcgcaaaacctcctgcgactggcggtacacggccattttggtggtctggtacacgggctcctgacgacccttgacgaacagtgcgaaacgcagttcctcctccgttgcggcgcagaaataggcgtgcacattgccgtccagatttttgcgcaccacggtgacgatggcgtttttcacaagctccacggcggcatcatcgaggccggcctcgaaaatgaggattttctcctttaactcctgcgggtttttcacgctgcgctttttcagcgcgcgcacggcggggataggctccgcgccggataccgcgccggaggggcgcaggcagatcataaacgcgttcatcgggtcatggtagatcatggggtacgccatctgcgcggtgtagccgcagtggaggcattcccagtcaaacatggattcgcgcagaatgtcgcggcgcagctcggggttttcctccgcattgacgcttgtgtacatctctaatttctgcgtctggccacaggcggggcagacaatatctttctgtatcttcgtagacatcaaattcactcactttctgcaaacggaacctgccccggcgcaaaaggtgggcgtttttccgcgcaaggaatcaagcgggcctcaggtccgccttttaacgttcattttaaagcgcattattcgtagggaacttcttcgctttcgtctttttcgggctcagtcgtttccggctgctcggcggcaggctctgccgcttccacggcttcgtctacggtaccgtccggcacgaatgcggcttcttcggcttccttttcctcttccggcgtgggagtgtactcctgcacgtcgtcaagggtgatctcctcttcttccgcttcgctgtcaccctcacgggtcagggcttccagcttctccacagcggcatcgatgcgcttctgggcggcttcaatgtcgatgcagtacggctccgtaacggtgttctctgcatcgcgcaggttgtggtagtagtagcggccgagagcgagataggcgcgctcctctgccttttcctcacagcggataacggtgcgcaggcgattcatgagcgcagtgcggcggttcttttcctctaccactttcacagcgccctgtacggcgccgccggcggtcttaaatgcggccgagatattcttcaaaaagttcatggtaaaactcctttcggtcaaaacgccaaggcgttcggtcatttcaagtttaaatacacctttattatatatcaaaaaagcgcaaaataacagccccaaaatgtaaataattccatataaatatgcagaaaagcggacatcgcaggacattataggacatcgtaggacatcttttgaacatttgttccgtgttcgattgacatttcggaaatttttgtttatgatgagagtcaacgaacacatgttctatttcgaaacgaaaggatcgtgactgctagtgaacgattacatttcctggggacaggagtatttggatgaggcggagcgcatcaaacggcggctgctcgtcatccgcgccgaacgcgactatgacattgcaggcgagcaggcccagcgtgtgcagatgctttacaccatgtatttggagtgccttgtaacaggccatgcgctgcaaaagcgcggaaggagggcggtttcgtgagcggatataagcccctttccgccggtgcacggcaggcgaagctactcagccgcatcggcgcagagacaaacaaaaatgagcttttagaagcgcaggttttgctgcggcgcgccatcggcaatgagctgactgaccgccagcgcgactgcgtgcagctttactacggcgagcggctgacaatgcaggagattgcggacgagctgcacctgacgcgcggcacggtgtgcaagcacctgcaaaaggcttcggcacgactttcgcacgctttgcagtattctgcgctcggcatgcgtgcggagcaaagccgcgaccgataacccccttgaaaagcctctccgattgtgctactatttgtacagacaactcagcatagatcacagcagatttatctgcggaaaggcttatactatggatttgattcaaattgacgaaaacgggctgcatctcgtttttgaaaagacggaagatttcggctgcaagctgctgcacttctctgcgctcccctttgacgagcgcagtctgctgcacgtgcacgaccgcgagacgggcggcttgcgcctgaacaccgagcgatacaatctcttggaaattatgatctccgggcaaaaccgaccgggcgagcgccacggcaacaaatacatttccacaagccccggcgaccgcatgaagctcgtctattttgcagacaagcacaacgagttcggccgcaagctggaaatccacatggaagataagacctgcggtattgaaacgatttcgcatatgcagttttataacggcgttcaggctgtacgctgctggacggacgtgaaaaatgtcggaacagagccgcaggggctggaatacgtttcctcctttgccctcaccggcatcgaaaaagagggtatgctttcgaccgacgacaaaatgcgcgtctggtacccgcacaacggctggcagcgcgaaatgcagtgggaaagctacactttcccgcagctcggtctcgcccgctgccagcccgaggaaattcagcgctcctctaaggtgattggtgcgacgaacatcggcaactggtctacaaaggaatacctgccgatgggactgcttgaaaacacggaaacgggctccaatttattctggcagatcgagcacaacggctcgtggcactgggaaatcagcgaccagatgggcaacttctacattcagttaagcggcccgacggaaacggaatcgcactggtggaagaatttgcagccgggcgaaacattcacgacagtgccggtgtgcgtgggctccacgctcggcaaatttgatgacgccatgggcgagctgacgaagtaccgccgcctgattcgccgcaaaaatgcggacaacgagaaactgtgcgtcattttcaacgactacatgaactgcctgtggggcaacccgacgacggcgaaagagctgccgctcatcgacgcggcgcacaaggcgggctgcgaatacttctgcgtggacgcaggctggtacgcggacggcttctggtgggacaacgtgggcgaatggctgccgagtaaggaacgtttcccgaacggccttgaggaagtgatggactacatccgctcgaagggtatgattccgggcgtgtggctcgaaattgaagtaatgggcatttgctgcccgaaggccgcgcgcgtgcacccggactggttcttcacacgccacggcaaaaaggtgcacgaccgcagccgctggcagctcgatttccgcaacccggaggtcgtggagcacgcaaatgaggtcgttgaccgccttgtgcgcgactaccacgtgggctacatcaagatggattacaacattgagccgggcatcggcacggaattaaacgccgacagcgtgggcgacggtttactcgggcacgagcgcgcatatcttgcttggctcgattccgtctttgcccgctaccccgaccttgtgattgaaaactgttcctcgggcggcatgcgcatcgactacgcgatgctctcgcgccacagcattcagtctacgagcgaccaggaggactacctgcgctatgcgtccattgcggcgaacgcgccctcgggtctgacgccggagcagagcgccatttggagctacccgatgaaaggcggcggacgcgaggaagccgtatttaacatgtgcaatgcactgatgcagcgcatccaccagagcggtcatttggcggaaattcctgaggaagacttcacgctcgtcaaggaagcgctcgattattacaaaacaatccgcggcgacatcaagcgcgcgctgccgttctggccgctcggtacgtcgcactttgcggacacgtgggtctcgctgggccttaaaaccgcgcacaaagcgtacatcaccgtgtggagacgcggcgcgccggacggccgctgcacgctgcccgtggatttcctgcgcggcaacgagcacgtaaatgtgcgctgcgcataccccgcaaaccacaacacgaaatgcgcatggaacccggagaagggcacgctgacggtagatttccccgcccccatctgcgccagaatttttgagctggaatattaaacctgcgcctgaacccgtacagaacaccgtgcaaaatcccgatgcagacgaagaaagacatctcgcggaagaagcgcggcgcaaggcggaggaagcgcaaaaggaagccgaagaagcgcggcagcgcgtggcggaagcccaaaagcgagcggagcgtgccgcacagaaaatggaagacacaatgaagcgcgccaagcttgcgcaggagcagttaaaaaaagcgcaggcagaggccgaaaagctcaaaaatcagaacaaaaagacgtaaaaaatcgcccgcaggcacggaaaacggttgcctgcgggcgatggtttttgaattttacttttcgatgtctttttcgacgttctgcggagcagtgggcgaagaagcgggctgcacgggcatctgcacggaagccgctgcgggtgctgaaacgggtgcgtaggcgctgcgcattttggcggtggtgaccatgtgctttgcgccctttgccatggcgatgatggtaggcacggcaccgatgacggcgaagagatagacctgtgcaaggccgccgaagtaatcgccggaatcgacatagccttctttgataagatccatcatggcgcggaacgcggtgaagaaatcggtctcatagaactgcgccgcggtgattgaccaatcggcctgatacgcaaggaagatcatcaggagcatcaaaacgcaggaaatgatgatgcccggtgtgccgagctttttgccgagcatctcgtagccctttaacgtgcagatggccatgacaagaccggaaacggcggcgacgtagccgagctgaccgacgatgacgatgacgacggcaccgatgagcgaaccgatgagcgcacctacgataccgggcaaaacctgctcctgcttctgcatttttgtctggttctgctgcgcggcggcttcgctgacttgcccgaagcacggttcacacaaaaggtacgcgttgccggagacggtgtaggcgtctacgccctccgggcgaccgcaatgctcgcagcaatttgtatacccgtttgttctgaggaacgtgatgagcgtatcaagcgcactgcgcagggcctctgccattttatctttattgccggagagcttcacaaggaaattgacgcggaaacgctgcacggtacagccgctcaaaaccttattcattttttcgagcggtttgaaatccgcggggttcggcgccatgcccatgcgcgagaccgaaagcgaaagcatatagtagacgtggttgttggcgttcggcataatggtgacggtatacccctgcacattgccgaaaacgccctctgcattcgggtcatacataaggccgttcacaccggccgctgccgcgagctttgccatttgacttttgttcatatcccaatcctccaaatgttgttctttacctgtattctactatattcaggttgattcgtcaatcgggtggcgtaggaagtttttgttgccgaactcgccggaatgtaccttaatggcgatgttgccgggcagcttgcaatcgagctgcttatacatttcaatgaccttttcaggggtgaccgtttttgtgaaataaaccttgggttttacgaaaactcctcctaagaattcgttatgtgcgggtgtgactccgcatacatttctataacattaaaagtttgctttaagtcaatatgcgttttggctaattttgcaccggtcaatttggtgtgtttaccggatttgcaagcagaatattcaaaaagtcttttcttccggaactttctgcggtaaaatagggaaaaacggagcatgatgcaggaaaggaagtattttgatgcagaaaccgcttgtcagcgtgatcgtgccgatctacaacgcacagaacaacattgtgcgctgcgtggagagcattcggcggcagacgtatgaaaatcttgaaatattgcttttgaacgacggctctaaggacgtgagctacccggtgtgcgagatgctctcgaaggcggacgcgcgcatcgttttgattgataaggaaaactccggcgtttcggcgacgcggaacctcggcatgcgcatggcgcggggcaaatatttgcagtttgtggacgcggacgacacactggagccgtttgcgacagaactcctcgttcagcgtgcggaggataacaacgccgatctcgtcattgcggcgtataaccgcattgtgccgtacaagccgaaaaaggaagcgaagcttgacccgcttttgccgaataaaaagaagcagctgacggagcggttggcgaaattccagacattcggctttctgccgatgggctttatgacaaaggaggaattcgcctgcgggctaatgcaggagccggcgagcttttactacggcgtgatgtggaacaagctttaccgcgccgatttggtgcgcgcacacacggacgtgcggtgcagcgaggaaatgacgtggagcgaagacctgtattttaatttgacgtacatacgctatgcagaacggttttttgcgctgaccgcgccaatctacaactactacgacaaccccggcagcgcggtgcatctgacgaaggtgcgcacggcgattacggcgcgcacggcgcttttcagctactacaaggaattatacgagcagcttgggctgtacgaagaaaacaagctgcagattttcaaatacctcatctcctcttcggaggcgtgagcgtggaaaggacgtttaacatggcattcgtactcgaccaaacaaaaccgaaccagaaatattttgaggcgatctgcggcattccgcacccctcaaccgaagaaaaagccgtaagcgactacgtgtgctccgttgcggaaaagctcggtttaaactacgagcaggacaaattttggaatgtcatcgtgcgcaagccggcttctaaagggtacgaagacgcgcctgtcgtgatgctggaggggcacatggacatggtatgcgtgaaaacacccgagagcacgcatgatttttcgcatgaccccattgagatttatgtcgacgacaagggcgacatccgcgcgaaggacacgacgctcggcgcggacgacggttacggcgtggcgtacatgctggctgtgatggaggaggatttcccccacccgccgctggagctcgtctttacggtgcaggaggaaaacggctgcaacggcgtggacgcgctggacgcgggcaagctgaaatccagaagaatgatcggccttgacgtgatgggcgagacggtggaatacacaagctgtgtttcctgctttacgagcgacctgatgaaactgacgaaaacgtgcgagacggagcctgcaaagggcgcggccctcatgctgacggtttccggcgtgcagccggtgcactccggcgcaatggtgcacccggagcagttcaatgcgattaagatcacggcgcgtttgcttaatgcgctttccgaaaacggtgcggtgtaccgactttccaccatggcgggcggcgaagcggaaaactatgcgccggtggagacgaaaacggtgatcctctgcgacgaaccggacactgtgaaggcgattttaaacggcgagcttgagaaaatcgaccgcgaattgcaggacggcaagcagaatttgacgcttgaaatccgcgatgcggcggcgaacgagatgctttcggatgccgatacgcaggcgattgtcgatttgatctacctgatgccctcgaacacggtggcaatccggaccgcgggcgaggagatgacggcgacgaacaacgtgggcacggtatcgttaaacggcggcgcatttgaactggtgatgtccgaccgtgcggcgagccgcagctttaaaaacagtgtgctcggccgcgtgaagcgcttggctgaactctgcggcttcacgcttgagacgagcctgcgctatgactcctgggaataccagccgaactcctacatgcgccgcatgacggccgacctgatgaaagaagtgtacggccacgagatgatggagacggcgtgcccgggcggacttgaatgctgctctttactgccgcgcatggaggggctggacattgtgatgttcgcgcccatcggcgaaaactgccacacgacagacgaatacatgaatctaaaatcgtttgaccgcgtgtatgatttcctcaaaacgctgcttggcaggttgaaataaatatttataatttgatataaatattaaaaagcacgcccctttccgatgaatttcgggaaccggcgtgctttacttatctgcggtctatggtgacaacggtgcgataatcggggtcgagcttttgaatttcggcctctaacttttgggtgatgacggcgtcgctgtccttcaccgaaaacggcacggctgcgtcgaacacgatattactgtgcgtaacgccccagatgacgcggaaatcgtgaatggtgacggtggggtacagcgcggtgacgagcgactgcactttgcggtgcagcgcatcggtgcgggcatcgccgacgataacggggtcaaggtgaatgaccatgtggatacagaggtcacggctgaaatcacgctcgatgttatcgatgagatcgtggctgacgagaatgtcgtttttggcatcgacctcacaatgggcagaggcaaaacagcggcccacgccgtagttgtgcacagcaaggtcatgcatgccgatgatgccgggatacgagagcattttctcggtaatggtctgtacgagttctccctccggcgcttggccgaggagaggatctgcggtgctgattgtaagctgcacgccggagaacacgatgaacagcgcaaccgccgcgcccatgtagccgtccaaattcacgccggtgagcatggtaatgacggcgccgagcagcacgacgcttgtggcgatgacatcgttgcggctgtccttggacgtggcttcgacggactcggatttgatcatgcggccgaccttgcggtaaaagaggcactgccagagctttgccagaatggaaatgacgagcacaacgagggcgacgggcgtgagggcgttttcctccggcgtgaggattttctcaatggacgacatgccgagctgaagcccgaggaaaatgacgatgaacgagacgatgacgcccgaaagatactcgatgcgcgcgtggccgaacgggtgcttttcatccgccggcttacttgccattttaaagccgatgagcgtgacgacggacgagccggaatctgtgaggttattgacggcatcggccgtgacggaaacactgtgaaaaaccgtgccgacgataatcttcatcaaaaaaagcagaaaatttgtgacgataccgacggcacccgccacgcggccgcatttgtcgcgcacggcaggatcctcggtatttttatgatcgcggacaaagattcgcaaaatgagatcggtcataacgaaaatctccttttggttagcggctgaaaacccgccgaatatgcaagcagagcttgaggaaagctcctcaaactctgcttttgggtgatttaattttgtaaatcggcttacttctgatcaaagaactcgacgatttcgccgcaggggccggtgcagaacgcaatgcgcgcggtgatgtcggaaagctctacatctttcggttcgatggtgatctcgtagccggcttcacggacacgggcgacacaaccgtcggtatcatctgttgcgagcgcgagatgggcaaacttgccttccggcggcacatcgttctcttccgggacgggcagcacttcgatgcagctgttgtccccggtggagatcatcatgcaggggtatttttcgtcgccccaccggcgcacgaccttgaggccgagcaggtcaagatagaacgtttttgctttttcaaactgcgcatacgtcggacgcagtgccacgtggtgaatgcctttgatcaagctcattttgttttcctcccaaaagtacattttataaagacagggtatcgagaccgataccctgcttgatttgtaattctaaattactttctgttgaagatggacatgatgtcggagaaggtatcatcgtcgctcttcgcggaagttgtgctgccggtgctcggcgcagacgggttgccgaaaaggtctgcatcgctgatagccggggctgcgggcttctgggtcttttcgccgttcatgccgtcgaagccggtcgcgataacggtgaccatcatctcgtcttccatcgtctcgtcgaatgcagcaccccaaatgatgtttgcatcctcgcttgcgcgctcggtgatgagctgagacgcgacatcgatctcgtcgagggcgatgtccggagaggacttgatgttgatgatgacgcccttagcgcctgcgatagcggtctcaagcagcggactggagatagccgcaacagctgcctgctctgccttgtccttgcctgccgcacggccgacacccatgtgtgcatagcctgcatcctgcatgatggacttgacatcctcgaagtcgaggttgacgatgcccgggatctggatgaggtcggaaatgctctgtacgccctgacggagaacgtcatcggcgatgctgaatgcgtttgcaagcgtaatgcgctgctcgctgacgagcttcagtctttcgttcgggatgacaacgagggaatcgacgtgcttgcgcagctctgcaatgcccttttctgcctgatccatacggcgcttgccttcaaaagcgaacggctttgtgacgatgccgacggtgagggcacccatatcctttgcaatctctgcaacgaccggtgctgcacctgtgccggtgccgccgcccataccggcggtgacgaagaccatgtctgcgcccttgagggtagctgcgatcagctcgcggctctcctcggcagcctttgcgccaacctccggcttggagcctgcgcctctgccctgtgtattcttttcaccgatctgcacgcgggtcttggcctgcgagcgggacagtgcctgacggtctgtattcatgcagacgagctcgacgtgctgcacgccggcatctgccatgcggtttacggcgtttccgccgccgccgcctacaccaacgaccttaatggtctggggggtctcttcaagcatgttatcaatttcaaacggcattttatgttcctccttgatttggtctttaaccgttaacgatccgcacggatccgggattttcattcatcacgccgcctccggattcaaaacggttttctctcgagcttcgatcaaatccgccggatagcgtcccataaagggccatacctttacagttctttacagtctttcagctttacatagcttcatacactataatttatcacttttcccgccaaaattcaagatgaaagcggcaaaaaagtgagcctttgcaaatatttttactttacagcggttttcgggaaatctatggtgccggggctgttcaaattacccgaaaaacttgtgaaaaacagggaaacgcttccaaattttacacgttttcggcgtcgcctgtgttgtcaccgctgtcgtctgctgcggggtccgtgatgccgtcggtatcacccgtgggattggcagtatcgccgttctccccgtcgccgtttacggtgctgtctgagccgtccgtgcggcctgcggcatcaccgggtgcgggcgtggcggagactgcggtgcggtcttcctcgtaaaacgccttatttttctccggcacaacggaaagatcgatggagccgacggtctcctcggtgatctcgccgctcgtttccatttgacgcagcatctcgaggccaaacgccattttgtaggacatgccgttcatgttgccgaaacggaacgtgatgcggttatcatacgtcagcgtaaggtcgtaggggtttgtcatgttgatctccgtgacgttctgcgtctcgtttttcgtcatctgctccgcgatggcgttatagacttctttgacggaatcatcgccgaacgtgatcgtttcgctgggctttgtgttcgtgggatttgccccgagcaccacagtcgtgccctcgcacggctgtgaagcgatctccagcaccttgccggaagcacttacatagagataacccgcgtcgcacgcgacggaaaacgccgtttcggcgggggtaatttcaatttcaatggtggaagggatatgctttacaatctttgcgctttcaatgtacgggaaacggtgctcaagcgctttttcgcggctttccgtttccagcagcacgaggttatcgccctcgttgatgccgcacacgcggcggatatcggcttcatcgtagccggcatctgacgtgaatgtgacgttgcccaccttgaaaaacacgaagatgcacaaaaggaccgcgccgatgacgacgacgctgaatgcgattatattgaacaggagcagcgccctgctttttttctttctcattgcgagcttttccgctgcgctgcgtggagtacgcgccatgttcagcctccgtttttcacgttttatctcttacttttcgctctatattcggtatacgtctgcgccgagcgcccttaagttctcttcaaagtgcgcatagccgcggtcgatgtactgcgggtgcatcacctgtgtttcgccctgtgccgcaaggcctgcgatgacaagcgccgcgccgccgcgcaaatcctctgccgagaccggcgcaccggaaagctgcggcacgccctctatgacagccaccttgccctctaacttgatcttcgcgccgagccggttgagctcaccggcatgcttatagcggctttcaaaaatgttctctacaaaaatactggtgccgtcccccaccgccgccatggtcatgacgggcgcctgagcatccgtaggaaagccggggtacggcatggtgcgcacgtgacgcacgggttttaatctctgcggtgcgcgcaataataattcgctgccggactgcatcacggcgcagccggtgctttcaaacgccgacaaaaccggcaaaaggtgctcgctgttgacatcacgcagaagcaccgtaccgcccgtgacggcggccgcggccatgtatgttgcggcggcgatgcggtccggcatgacggtgtatgtgctgccgtaaaggtgcggcacgccttcgatgacgatgcagctttcgcccgcgccgtgaatgtgtgcgccgcagcggtttaagtacccggcgagatcctcaatctccggctcacgcgcggcgtttgtgagcaccgttgtgcccttggcgcgggcagcggcgagcagcaggttttccgttgcgccgacgcttggaaacgcaagcgagacgaacccgccgcgcaacccgccgggcgcggtgcacagcaggcagccgtgctcttcgcgaatttctgcgccgagcttgcgcaacgcctccaaatgcagatcgatgggccttgggccgagctcgcacccgccgggaaacgacaatcttgcttcgccgcagcgggacagaatcgcgcccaaaaacacgatggacgagcgcatctcccgcatgaggctgtgaggaatatcgcagcgggtcatgccgtccgcgcagaccgttaaggtatcgcccgtgtgggtacagtggcatccgagccgccgcaaaatggtgactgccgtgtgcacatcggacagtgcggggcagttgtgcagcaccgtttcgccctcacagaggagagccgccgcaagcagcggcagcgtgctgtttttcgcgccctgcacggtgagctcgccgcaaaggcgattgccgccgcgcaccaaaaccgcttcctgccgggcacagcccgtatgttccagttcatattcttccatatacacaccccgtattctgtaaggcggcagaaagcgcgcctttggatacggtcatagtatgcagcggaaaaattttggggtatgtccgcctgaaatgcgttttacggtttacttttttgctttttcgcaaatgatgttgtaaatgcggtcatttgcgtccagcacggccatggctttcgcgtttttgccgatctcttcggcgcggcccggctcggaaaggatggtgttcaccgtttcccacagcttttcaccggtgaggtctttttcttcgatcatcacggcggcatcgcggttcacgagcgccatggc

SEQ ID NO: 2 - RNA, 1382 nt, Copromonas sp.
  source 1..1382: mol_type = rRNA
tttcgcttgggtcactggcttcgggcatttccgactcccatggtgtgacgggcggtgtgtacaagacccgggaacgtattcaccgcagcattctgatctgcgattactagcgattccagcttcatgtagtcgagttgcagactacaatccgaactgagacgttatttttgagatttgctccccctcacaggctcgcttctctttgtttacgccattgtagcacgtgtgtagcccaaatcataaggggcatgatgatttgacgtcatccccaccttcctccaggttatccctggcagtctcatcagagtgcccagcttgacctgctggctactgacaataggggttgcgctcgttgcgggacttaacccaacatctcacgacacgagctgacgacaaccatgcaccacctgtcacctctgtcccgaaggaggagcccgattaaaggcttttcagagggatgtcaagatttggtaaggttcttcgcgttgcttcgaattaaaccacatgctccaccgcttgtgcgggtccccgtcaattcctttgagtttcattcttgcgaacgtactccccaggtggaatacttattgcgtttgcggcggcaccgatgagctttgctcaccaacacctagtattcatcgtttacggcgtggactaccagggtatctaatcctgtttgctccccacgctttcgagcctcaacgtcagttactgtccagtaagccgccttcgccactggtgttcttcctaatatctacgcatttcaccgctacactaggaattccgcttacctctccagcactctagcaacacagtttccaaagcagttcccaggttgagcccgggtatttcacttcagacttgcgtcgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagagtttcctccattgtgcaatatcccccactgctgcctcccgtaggagtctgggccgtgtctcagtcccaatgtggccggtcaccctctcaggtcggctactgatcgtcgccttggtgggccgttaccccaccaactagctaatcagacgcgggtccatctcataccaccggagtttttcacaccgtaccatgcggtactgtgcgcttatgcggtattagcagtcatttctaaccgttatccccctgtatgaggcaggttacccacgcgttactcacccgtccgccactcggtatctagaaatccatccgaaaacttcaatcaaaatacctcgtccgac

SEQ ID NO: 3 - RNA, 1401 nt, Copromonas sp.
  source 1..1401: mol_type = rRNA
tcgacagctccctgctttcgcttgggtcactggcttcgggcatttccgactcccatggtgtgacgggcggtgtgtacaagacccgggaacgtattcaccgcagcattctgatctgcgattactagcgattccagcttcatgtagtcgagttgcagactacaatccgaactgagacgttatttttgagatttgctccccctcgcaggctcgcttctctttgtttacgccattgtagcacgtgtgtagcccaaatcataaggggcatgatgatttgacgtcatccccaccttcctccaggttatccctggcagtctcatcagagtgcccagcttgacctgctggctactgacaataggggttgcgctcgttgcgggacttaacccaacatctcacgacacgagctgacgacaaccatgcaccacctgtcacctctgtcccgaaggaggagcccgattaaagacttttcagagggatgtcaagatttggtaaggttcttcgcgttgcttcgaattaaaccacatgctccaccgcttgtgcgggtccccgtcaattcctttgagtttcattcttgcgaacgtactccccaggtggaatacttattgcgtttgcggcggcaccgatgagctttgctcaccaacacctagtattcatcgtttacggcgtggactaccagggtatctaatcctgtttgctccccacgctttcgagcctcaacgtcagttactgtccagtaagccgccttcgccactggtgttcttcctaatatctacgcatttcaccgctacactaggaattccgcttacctctccagcactctagcaacacagtttccaaagcagttcccaggttgagcccgggtatttcacttcagacttgcgtcgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagagtttcctccattgtgcaatatcccccactgctgcctcccgtaggagtctgggccgtgtctcagtcccaatgtggccggtcaccctctcaggtcggctactgatcgtcgccttggtgggccgttaccccaccaactagctaatcagacgcgggtccatctcataccaccggagtttttcacaccgtaccatgcggtactgtgcgcttatgcggtattagcagtcatttctaaccgttatccccctgtatgaggcaggttacccacgcgttactcacccgtccgccactcggtatcttgaaatccatccgaaaacttcaatcaaaatacctcgttcgacttgc

SEQ ID NO: 4 - RNA, 1394 nt, Copromonas sp.
  source 1..1394: mol_type = rRNA
gcaagtcgaacgaggtaaatgagatgaagttttcggatggattcttatttaccgagtggcggacgggtgagtaacgcgtgggtaacctgcctcatacagggggataacgattggaaacgattgctaataccgcataagcgcacagtaccacatggtacagtgtgaaaaactccggtggtatgagatggacccgcgtctgattagctagttggtgaggtaacggcccaccaaggcaacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaatacccgggctcaacctgggaactgctttggaaactgtatggctagagtgctggagaggtaagcggaattcctagtgtagcggtgaaatgcgtagatattaggaagaacaccagtggcgaaggcggcttactggacagtaactgacgttcaggctcgaaagcgtggggagcaaacaggattagataccctggtagtccacgccgtaaacgatgaatactaggtgtcgggggacaaagtctttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaaacactcttaatcgagtgcctccttcgggacagaggagacaggtggtgcatggttgtcgtcagctcgtgtcgtgagatgttgggttaagtcccgcaacgagcgcaacccctattgtcagtagccagcaggtaaagctgggcactctgatgagactgccagggataacctggaggaaggtggggatgacgtcaaatcatcatgccccttatgatttgggctacacacgtgctacaatggcgtaaacaaagagaagcgaagctgtgaggcagagcaaatctcaaaaataacgtctcagttcggattgtagtctgcaactcgactacatgaagctggaatcgctagtaatcgcggatcagaatgccgcggtgaatacgttcccgggtcttgtacacaccgcccgtcacaccatgggagtcggaaatgcccgaagccagtgacccaagcgaaagcagggag

SEQ ID NO: 5 - RNA, 1403 nt, Hungatella sp.
  source 1..1403: mol_type = rRNA
gcaagtcgagcgaagcgatcgagatgaagttttcggatggattcctgattgactgagcggcggacgggtgagtaacgcgtgggtaacctgcctcatacagggggataacagttagaaatgactgctaataccgcataagcgcacagtaccacatggtacggtgtgaaaaactccggtggtatgagatggacccgcgtctgattagctggttggtggggtaacggcccaccaaggcgacgatcagtagccgacctgagagggtgatcggccacattgggactgagacacggcccaaactcctacgggaggcagcagtggggaatattgcacaatgggggaaaccctgatgcagcaacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcagcgcaagtctgaagtgaaagcccggggctcaaccccggaatggctttggaaactgtgcagctagagtaccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcggtaaacgatgaatgctaggtgttggtgagcaaagctcatcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatcccgatgaaagatccttaaccggattccctcttcggagcatcggagacaggtggtgcatggttgtcgtcagctcgtgtcgtgagatgttgggttaagtcccgcaacgagcgcaacccttatcctcagtagccagcagtaagatgggcactctgtggagactgccagggataacctggaggaaggcggggatgacgtcaaatcatcatgccccttatgatttgggctacacacgtgctacaatggcgtaaacaaagggaagcgagaccgcgaggtggagcaaatcccaaaaataacgtctcagttcggattgcaggctgcaactcgcctgcatgaagctggaatcgctagtaatcgcggatcagaatgccgcggtgaatacgttcccgggtcttgtacacaccgcccgtcacaccatgggagtcgataacgcccgaagtcagtgacctaaccgtaaggaaggagctgccgaagg

SEQ ID NO: 6 - RNA, 1384 nt, Dysosmobacter sp.
  source 1..1384: mol_type = rRNA
gccctccttacggttaggctacggacttcgggtgttcccgactctcatggtgtgacgggcggtgtgtacaaggcccgggaacgtattcaccgtggtatgctgacccacgattactagcgattccgacttcacgcaggcgggttgcagcctgcgatccgaactgggacggctttttggggttcgctctgcctcgcggctttgcatccctttgttgaccgccattgtattacgtgtgtagcccaggtcataaggggcatgatgatttgacgtcgtccccaccttcctccgttttgtcaacggcagtctcgctagagtgctcttgcgtagcaactaacaataggggttgcgctcgttgcgggacttaacccaacatctcacgacacgagctgacgacaaccatgcaccacctgtctctgctttccccgaagggcacctaatgtatctctacttcgttagcaggatgtcaagacctggtaaggttcttcgcgttgcttcgaattaaaccacataatccaccgcttgtgcgggcccccgtcaattcctttgagtttcaaccttgcgatcgtactccccaggtgggatacttattgtgttaactgcggcacgcagggggtcagtccccgcacacctagtatccatcgtttacagcgtggactaccagggtatctaatcctgttcgctccccacgctttcgcgcctcaccgtcagttaccgtccagtaatccgccttcgccactggtgttcttccttatatctacgcatttcaccgctacacaaggaattccgattacctctccggcactcaagacatacagtttcaaatgcagtttcagggttaagccctgagatttcacatctgacttgcatgcccggctgcacgccctttacacccagtaaatccggacaacgcttgccacctacgtattaccgcggctgctggcacgtagttagccgtggcttattcaagtggtaccgtcttctactcttcccacttaaaagaagtttacaacccgaaagccttcttccttcacgcggcgttgctgggtcaggcttgcgcccattgcccaatattccccactgctgcctcccgtaggagtctgggccgtatctcagtcccaatgtggccggtcaacctctcagtccggctactgatcgtcgacttggtgggccgttaccccgccaactatctaatcagacgcgaggccatctttcagcgataaatctttggcatttcgaccatgcgatcaaaatgcgtcatgcggtattagcagtcgtttccaactgttgtccccctctgaaaggcaggttcctcacgcgttactcaccagtccgccactaagcattcctatcatgtggccgaaacctcaatcaagggtgctccgtt

SEQ ID NO: 7 - RNA, 1399 nt, Oliverpabstia sp.
  source 1..1399: mol_type = rRNA
cgaacgaagcactttggacagattcttcggatgaagtccttagtgactgagtggcggacgggtgagtaacgcgtgggtaacctgcctcatacagggggataacagttagaaatggctgctaataccgcataagcgcacggtactgcatggtacagtgtgaaaaactccggtggtatgagatggacccgcgttggattagctagttggcagggtaacggcctaccaaggcgacgatccatagccggcctgagagggtggacggccacattgggactgagacacggcccagactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggaatggcaagtctgatgtgaaaggccggggctcaaccccgggactgcattggaaactgtcaatctagagtaccggaggggtaagtggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatcgatctgaccggactgtaatgggtcctttcccttcggggacagagaagacaggtggtgcatggttgtcgtcagctcgtgtcgtgagatgttgggttaagtcccgcaacgagcgcaacccttatcctcagtagccagcaagtgaagttgggcactctgtggagactgccagggataacctggaggaaggtggggatgacgtcaaatcatcatgccccttatgatttgggctacacacgtgctacaatggcgtaaacaaagggaagcgatcacgcgagtgtgagcaaatctcaaaaataacgtctcagttcggattgtagtctgcaactcgactacatgaagctggaatcgctagtaatcgcaggtcagcatactgcggtgaatacgttcccgggtcttgtacacaccgcccgtcacaccatgggagtcagtaacacccgaagccggtgacctaaccgaaaggaaggagccgtcgaa

SEQ ID NO: 8 - RNA, 1394 nt, Copromonas sp.
  source 1..1394: mol_type = rRNA
gcaagtcgaacgaggtattttgattgaagttttcggatggatttcaagataccgagtggcggacgggtgagtaacgcgtgggtaacctgcctcatacagggggataacggttagaaatgactgctaataccgcataagcgcacagtaccgcatggtacggtgtgaaaaactccggtggtatgagatggacccgcgtctgattagctagttggtggggtaacggcccaccaaggcgacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctgaagtgaaatacccgggctcaacctgggaactgctttggaaactgtgttgctagagtgctggagaggtaagcggaattcctagtgtagcggtgaaatgcgtagatattaggaagaacaccagtggcgaaggcggcttactggacagtaactgacgttgaggctcgaaagcgtggggagcaaacaggattagataccctggtagtccacgccgtaaacgatgaatactaggtgttggtgagcaaagctcatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaaaagtctttaatcgggctcctccttcgggacagaggtgacaggtggtgcatggttgtcgtcagctcgtgtcgtgagatgttgggttaagtcccgcaacgagcgcaacccctattgtcagtagccagcaggtcaagctgggcactctgatgagactgccagggataacctggaggaaggtggggatgacgtcaaatcatcatgccccttatgatttgggctacacacgtgctacaatggcgtaaacaaagagaagcgagcctgcgagggggagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagccagtgacccaagcgaaagcagggag

SEQ ID NO: 9 - RNA, 1379 nt, unidentified
  source 1..1379: mol_type = rRNA; note = Acutalibacter nov. genus
gcaagtcgaacggagttgaaaggagcttgcttttttcaactcagtggcggacgggtgagtaacgcgtgagtaacctgcctttcagagggggataacgttctgaaaagaacgctaataccgcataagattgtagtttcgcatggagcagcaatcaaaggagcaatccgctgaaagatggactcgcgtccgattagatagttggcggggtaacggcccaccaagtcgacgatcggtagccggactgagaggttgatcggccacattgggactgagacacggcccagactcctacgggaggcagcagtgagggatattgggcaatgggggaaaccctgacccagcaacgccgcgtgagggatgacggttttcggattgtaaacctctgtcctctgtgaagatagtgacggtagcagaggaggaagctccggctaactacgtgccagcagccgcggtaatacgtagggagcaagcgttgtccggatttactgggtgtaaagggtgcgtaggtggattggcaagtcagaagtgaaatccatgggcttaacccatgaactgcttttgaaactgttagtcttgagtgaagtagaggtaggcggaattcccggtgtagcggtgaaatgcgtagagatcgggaggaacaccagtggcgaaggcggcctactgggctttaactgacgctgaggcacgaaagtgtgggtagcaaacaggattagataccctggtagtccacactgtaaacgatgattactaggtgtggggggtctaaccccctccgtgccggagttaacacaataagtaatccacctggggagtacgaccgcaaggttgaaactcaaaggaattgacgggggcccgcacaagcagtggagtatgtggtttaattcgacgcaacgcgaagaaccttaccaggtcttgacatccaactaacgaagcagagatgcgttaggtgcccttcggggaaagttgagacaggtggtgcatggttgtcgtcagctcgtgtcgtgagatgttgggttaagtcccgcaacgagcgcaacccttgtgattagttgctacgcaagagcactctaatcagactgccgttgacaaaacggaggaaggtggggacgacgtcaaatcatcatgccctttatgacctgggctacacacgtactacaatggtcgttaacagagggaagcaataccgcgaggtggagcaaaaccctaaaaacggtctcagttcagattgtaggctgcaactcgcctacatgaagttggaattgctagtaatcgcggatcagcatgccgcggtgaatacgttcccgggccttgtacacaccgcccgtcacaccatgggagccggtaatacccgaagtcagtagtttaaccgtaaggagaac

SEQ ID NO: 10 - RNA, 1381 nt, unidentified
  source 1..1381: mol_type = rRNA; note = Merdisoma Aerosacheriphilus
gcaagtcgaacggacgatgaagagcttgcttttcagagttagtggcggacgggtgagtaacgcgtgggtaacctgcctcatacagggggatagcagctggaaacggctgataaaaccgcataagcgcacagcatcgcatgatgcagtgtgaaaaactccggtggtatgagatggacccgcgtctgattagctggttggtgaggtaacggcccaccaaggcgacgatcagtagccggcctgagagggtgaccggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtaggcggcggagcaagtcagaagtgaaagcccggggctcaaccccgggacggcttttgaaactgccctgcttgatttcaggagaggtaagcggaattcctagtgtagcggtgaaatgcgtagatattaggaggaacaccagtggcgaaggcggcttactggactgacaatgacgctgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggccttgacatcccggtgaccgttccgtaatggggacctctcttcggagcaccggtgacaggtggtgcatggttgtcgtcagctcgtgtcgtgagatgttgggttaagtcccgcaacgagcgcaacccctatgttcagtagccagcaggtaaagctgggcactctggacagactgccggggataacccggaggaaggcggggatgacgtcaaatcatcatgccccttacggcctgggctacacacgtgctacaatggcgtaaacaaagggaagcgagagggtgacctggagcgaatcccaaaaataacgtcccagttcggactgtagtctgcaacccgactacacgaagctggaatcgctagtaatcgcgaatcagcatgtcgcggtgaatacgttcccgggtcttgtacacaccgcccgtcacaccatgggagttggaaatgcccgaagtcagtgacctaaccgcaagggaggag

SEQ ID NO: 11 - RNA, 1402 nt, Longicatena sp.
  source 1..1402: mol_type = rRNA
gcaagtcgaacgaagtcttgaggaagcttgcttccaaagagacttagtggcgaacgggtgagtaacacgtagggaacctgcccatgtgcccgggataacagatggaaacgtctgctaaaaccggataggcagtgagacggcatcgtcttgctgttaaaggagcttcggctctgaacatggatggacctgcggcgcattagcttgttggcggggtaacggcccaccaaggcaatgatgcgtagccgacctgagagggtgaacggccacattgggactgagacacggcccaaactcctacgggaggcagcagtagggaattttcgtcaatggagggaactctgaacgagcaatgccgcgtgagtgaagaaggtcttcggatcgtaaagctctgttgcaagagaaaaacggcacatacaggaaatggtatgtgagtgatggtatcttgccagaaagccacggctaactacgtgccagcagccgcggtaatacgtaggtggcaagcgttatccggaatgattgggcgtaaagggtgcgtaggtggtgcagtaagtctgaagtaaaagccagcagctcaactgctgtaagctttggaaactgctgtactagagtgcaggagagggcgatggaattccatgtgtagcggtaaaatgcgtagatatatggaggaacaccagtggcgaaggcggtcgcctggcctgcaactgacactgatgcacgaaagcgtggggagcaaataggattagataccctagtagtccacgccgtaaacgatgagaactaagtgttggagagattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggatataaatattctagagatagaaagatagctatatatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagcgatgtgaagcgaatctcataaaggccgtctcagttcggattgaagtctgcaactcgacttcatgaagtcggaatcgctagtaatcgcagatcagcatgctgcggtgaatacgttctcgggccttgtacacaccgcccgtcaaaccatgggagttggtaatacccgaagccggtggcataaccgcaaggagtgagc

SEQ ID NO: 12 - RNA, 1390 nt, Enterocloster sp.
  source 1..1390: mol_type = rRNA
gtcgaacgaagcgattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcacgtgaaggtgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggaggga

SEQ ID NO: 13 - RNA, 1241 nt, unidentified
  source 1..1241: mol_type = rRNA; note = Rhizobiaceae nov. genus
tcggacgggaagtggtgtttccagtggcggacgggtgagtaacgcgtaanaacctgcccttgggaggggaacaacagctggaaacggctgctaataccccataggctgaggagcaaaaggaggaatccgcccaaggaggggctcgcgtctgattagttagttggtgaggcaatggcttaccaaggcgacgatcagtagctggtccgagaggatgatcagccacactgggactgagacacggcccagactcctacgggaggcagcagtggggaattttccgcaatgggcgaaagcctgacggagcaatgccgcgtgaaggcagaaggcccacgggtcatgaacttcttttctcggagaagaaacaatgacggtatctgaggaataagcatcggctaactctgtgccagcagccgcggtaagacagaggatgcaagcgttatccggaatgattgggcgtaaagcgtctgtaggtggcttttcaagtccgccgtcaaatcccagggctcaaccctggacaggcggtggaaactaccaagctggagtacggtaggggcagagggaatttccggtggagcggtgaaatgcgttgagatcggaaagaacaccaacggcgaaagcactctgctgggccgacactgacactgagagacgaaagctaggggagcaaatgggattagataccccagtagtcctagccgtaaacgatggatactaagtgctgtgcgtatcgacccgcgcagtgctgtagctaacgcgttaagtatcccgcctggggagtacgttcgcaagaatgaaactcaaaggaattgacgggggcccgcacaagcggtggagcatgtggttcaattcgatgcaaagcgaagaaccttaccagggcttgacatgccgtgaatcctcccgaaagagaggagtgccttcgggaacgcggacacaggtggtgcatggctgtcgtcagctcgtgccgtaaggtgttgggttaagtcccgcaacgagcgcaaccctcgtgtttagttgccagcattgagtttggaaccctgaacagactgccggtgataagccggaggaaggtgaggatgacgtcaagtcatcatgccccttacgccctgggcgacacacgtgctacaatgaccgggacaaagggtcgcgaccccgcgagggcaagctaacctcaaaaacccggcctcagttcggattgcaggctgcaactcgcctgcatgaagccggaatcgctagtaatcgcc

SEQ ID NO: 14 - RNA, 1385 nt, unidentified
  source 1..1385: mol_type = rRNA; note = Anaerotignum nov sp.
tcgagcggagatatttagaaagcttgctttttgaatatcttagcggcggacgggtgagtaacgtgtgggcaacctgcctcatacagagggataatcatgtgaaaacgtgactaataccgcatgtcattactggaaggcatcttccggtaagaaaaggagcaatccggtatgagatgggcccgcatctgattagctagttggtgagataatagcccaccaaggcgacgatcagtagccgacctgagagggtgatcggccacattgggactgagacacggcccaaactcctacgggaggcagcagtggggaatattgcacaatgggggaaaccctgatgcagcaacgccgcgtgaaggaagacggttttcggattgtaaacttctatcaatagggacgaagaaagtgacggtacctaaataagaagccccggctaactacgtgccagcagccgcggtaatacgtagggggcaagcgttatccggaattactgggtgtaaagggtgagtaggcggcacggcaagtaagatgtgaaagcccggggcttaactccgggattgcattttaaactgctgagctagagtacaggagaggaaagcggaattcctagtgtagcggtgaaatgcgtagatattaggaagaacaccagtggcgaaggcggctttctggactggaaactgacgctgaggcacgaaagcgtggggagcgaacaggattagataccctggtagtccacgccgtaaacgatgagtgctaggtgtcggaggggaaccttcggtgccgcagcaaacgcaataagcactccacctggggagtacgatcgcaagattgaaactcaaaggaattgacgggggcccgcacaagcggtggagcatgtggtttaattcgaagcaacgcgaagaaccttaccaaggcttgacatcccgatgaccggttcagagatgaaccttctcttcggagcatcggagacaggtggtgcatggttgtcgtcagctcgtgtcgtgagatgttgggttaagtcccgcaacgagcgcaacccttatctttagtagccagcattaagatgggcactctaaagagactgccgtggataacacggaggaaggtggggatgacgtcaaatcatcatgccccttatgtcttgggcaacacacgtgctacaatggacggaacaaagtgaagcgagaccgcgaggtggagcaaagcacaaaaacccgttctcagttcggattgtagtctgcaactcgactacatgaagctggaatcgctagtaatcgcgaatcagaatgtcgcggtgaatacgttcccgggccttgtacacaccgcccgtcacaccatgggagttggaagcacccgaagcctgtgacccaacctgaaaaggagggagc

SEQ ID NO: 15 - RNA, 1400 nt, unidentified
  source 1..1400: mol_type = rRNA; note = Frisingococcus sp.
agtcgaacgaagcactttggatggatttcttcggattgatatccttagtgactgagtggcggacgggtgagtaacgcgtgggtaacctgcctcatacagggggataacagttagaaatggctgctaataccgcataagcgcacagtaccacatggtaccgtgtgaaaaactccggtggtatgagatggacccgcgtctgattagctagttggcagggtaacggcctaccaaggcgacgatcagtagccgacctgagagggtgatcggccacattgggactgagacacggcccaaactcctacgggaggcagcagtggggaatattgcacaatgggggaaaccctgatgcagcgacgccgcgtgaaggaagacgtatttcggtatgtaaacttctatcagcaaggaagatgatgacggtacttgactaagaagccccggctaactacgtgccagcagccgcggtaatacgtagggggcaagcgttatccggatttactgggtgtaaagggagcgtaggcggccatgcaagtcagaagtgaaagcccggggctcaaccccgggactgcttttgaaactgtaaggctagattgccggagaggtaagcggaattcctagtgtagcggtgaaatgcgtagatattaggaggaacaccagtggcgaaggcggcttactggacggtgaatgacgctgaggctcgaaagcgtggggagcaaacaggattagataccctggtagtccacgctgtaaacgatgaatactaggtgttggggtccaaaaggacttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaggtcttgacatccccctgacggcagagtaatgtctgcgttcctacgggacaggggagacaggtggtgcatggttgtcgtcagctcgtgtcgtgagatgttgggttaagtcccgcaacgagcgcaacccctatttttagtagccagcggtccggccgggcactctagagagactgccagggataacctggaggaaggcggggatgacgtcaaatcatcatgccccttatgatctgggctacacacgtgctacaatggtggtaacaaagtgaagcgagcctgcgagggggagccaatcacagaaaagccatctcagttcggattgtagtctgcaactcgactacatgaagctggaatcgctagtaatcgcgaatcagcatgtcgcggtgaatacgttcccgggtcttgtacacaccgcccgtcacaccatgggagttggatatgcccgaagtcagtgacccaaccgtaaggagggagcttccg

SEQ ID NO: 16 - RNA, 1382 nt, Anaerotignum sp.
  source 1..1382: mol_type = rRNA
gcagtcgacgaagataacttaaaatgagagcttcggcaggatttttatttatcttagtggcggacgggtgagtaacgtgtgggcaacctgccctatactggggaataatcactggaaacggtgactaataccgcatgtcattacggaagggcatccttctgtaagaaaaggagtaattcggtataggatgggcccgcatctgattagctagttggtgagataacagcccaccaaggcgacgatcagtagccgacctgagagggtgatcggccacattgggactgagacacggcccaaactcctacgggaggcagcagtggggaatattgcacaatgggggaaaccctgatgcagcaacgccgcgtgaaggaagaaggttttcggatcgtaaacttctatcaacagggacgaagaaagtgacggtacctgaataagaagccccggctaactacgtgccagcagccgcggtaatacgtagggggcaagcgttatccggaattactgggtgtaaagggagcgtaggcggcacgccaagccagatgtgaaagcccgaggcttaacctcgcggattgcatttggaactggcgagctagagtacaggagaggaaagcggaattcctagtgtagcggtgaaatgcgtagatattaggaagaacaccagtggcgaaggcggctttctggactgaaactgacgctgaggctcgaaagcgtggggagcaaacaggattagataccctggtagtccacgccgtaaacgatgagtgctaggtgtcggggaggaatcctcggtgccgcagctaacgcaataagcactccacctggggagtacgaccgcaaggttgaaactcaaaggaattgacgggggcccgcacaagcggtggagcatgtggtttaattcgaagcaacgcgaagaaccttaccaaggcttgacatcccgatgaccgctctagagatagagcttctcttcggagcatcggtgacaggtggtgcatggttgtcgtcagctcgtgtcgtgagatgttgggttaagtcccgcaacgagcgcaacccttatccttagtagccatcattgagttgggcactctagggagactgccgtggataacacggaggaaggtggggatgacgtcaaatcatcatgccccttatgtcttgggctacacacgtgctacaatggctggtaacagagtgaagcgagacggcgacgttaagcaaatcacaaaaacccagtcccagttcggattgtagtctgcaactcgactacatgaagctggaatcgctagtaatcgcgaatcagaatgtcgcggtgaatacgttcccgggccttgtacacaccgcccgtcacaccatgggagtggaagcacccgaagtcggtgacctaa

SEQ ID NO: 17 - RNA, 1230 nt, Blautia wexlerae
  source 1..1230: mol_type = rRNA
ttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtccttaatcggaccttttcttcgggacaggggagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactaca

(19) United States
(12) Patent Application Publication
(10) Pub. No.: US 2026/0294985 A1
(43) Pub. Date: Oct. 1, 2026

(54) COMPOSITIONS COMPRISING BACTERIA CONSORTIA

(71) Applicant: PHARMABIOME AG, SCHLIEREN (CH)

(72) Inventors: TOMAS DE WOUTERS, ZURICH (CH); GABRIEL LEVENTHAL, PFEFFINGEN (CH)

(21) Appl. No.: 19/479,508

(22) PCT Filed: Apr. 26, 2024
(86) PCT No.: PCT/EP2024/061647

§ 371 Date: Oct. 28, 2025

(30) Foreign Application Priority Data
Apr. 28, 2023 (EP) 23170659.9
Oct. 31, 2023 (EP) 23207180.3

(51) Int. Cl. A61K 35/741; A61K 35/00; A61K 35/744; A61K 35/745; A61K 35/747; A61P 1/00

(52) U.S. Cl. CPC A61K 35/741; A61K 35/744; A61K 35/745; A61K 35/747; A61P 1/00; A61K 2035/115

(57) ABSTRACT

The present invention provides consortia of bacterial strains which reflect the intestinal microbiome complexity and increase desirable end metabolites production and their uses.

### FIELD OF THE INVENTION

The present invention relates to the field of microbiology. It provides compositions comprising consortia of bacterial strains and uses thereof.

### BACKGROUND OF THE INVENTION

Intestinal microbiome-based live biotherapeutic products (LBPs) are emerging as a novel modality to treat a large number of chronic diseases. The therapeutic objective is to induce a targeted modulation of the intestinal microbiota by administering live microorganisms to reverse dysbiosis and promote recovery.

Fecal microbiota transfer (FMT) from healthy donors is currently the most successful method of restoring diseased microbiota to a healthy state. However, it is not fully understood what the specific properties of a healthy microbiota are and how these might promote recovery, resulting in variable efficacy of FMT for different diseases. Moreover, the potential for adverse events with FMT raises critical safety concerns.

Defined bacterial consortia that are well-characterized represent a safer and more controlled alternative to FMT. Because a healthy microbiota typically contains hundreds of strains, simply recreating the full taxonomic diversity in a defined product is intangible. The complexity can, however, be reduced by removing functionally redundant strains and focusing on those microbial functions that are key for the specific therapeutic target. While intuitive in principle, translating this functional concept into a defined consortium is not straightforward, because most of the intestinal microbiota remains uncultured and uncharacterized. In addition, mixing together microorganisms does not guarantee them to interact with one another and to occupy functional niches that require microbial networking. Product stability, standardization and performance of important functions can therefore not always be guaranteed.

Then, there is a need to design alternative and specific consortia of bacterial species which can reflect the intestinal microbiome complexity and which can be effectively used to prevent or treat intestinal dysbiosis and diseases or disorders related to such condition.

In addition, it is known in the art that intestinal dysbiosis impairs the production of short-chain fatty acids (SCFA), organic fatty acids with one to six carbons that are produced within the intestinal lumen by bacterial fermentation of undigested dietary carbohydrates. Acetate, propionate, and butyrate are the most abundant SCFA produced in the gastrointestinal tract (GIT).

Among SCFA studies, there have been many investigations into the effects of butyrate and its potential applications in human health. Butyrate is known to have beneficial effects on epithelial barrier function and overall gut health. It is a cellular mediator regulating multiple functions of gut human and microbial cells including gene expression, immune modulation and oxidative stress reduction. Mammalian cells do not produce significant amounts of butyrate, so that the only significant sources are the intestinal microbiome and ingestion of dairy products.

There is thus a need to develop specific consortia of bacterial species which can both reflect the intestinal microbiome complexity and increase desirable end metabolites production such as butyrate, to prevent or treat intestinal dysbiosis and related diseases or disorders.

### SUMMARY OF THE INVENTION

Here, the inventors present consortia of bacterial strains in which the overall function of an intestinal microbiome is divided among the bacterial strains based on the principle of ‘division of labor’. The inventors particularly selected anaerobic intestinal bacteria that cover the essential elements of carbohydrate metabolism in the large intestine.

Using continuous co-culture fermentations, the inventors confirmed the reproducible establishment of a compositional and metabolic equilibrium with complete carbohydrate fermentation akin to a healthy intestinal microbiome in such consortia. Such co-cultured consortium performs differently than an equivalent mix of identical but individually cultivated bacterial strains.

The inventors have particularly designed bacterial consortia reflecting the carbohydrate degradation metabolic network of the human intestinal microbiome which is stable over time and avoids accumulation of intermediate metabolites while reliably producing end metabolites. The inventors surprisingly found that such consortia are able to enhance butyrate production, without destabilization of the consortium. The shift towards butyrate can be achieved by specifically selecting the bacterial strains fulfilling a butyrate producing function of the said metabolic network, such as bacterial strains Agathobacter rectalis and Anaerostipes caccae.

The inventors show a synergistic effect of the combination of Agathobacter rectalis and Anaerostipes caccae but also of Anaerobutyricum hallii and A. rectalis on butyrate production. The metabolic profile of the co-culture goes beyond the sum of butyrate production by each strain taken in isolation.

In a first aspect, the invention concerns a composition comprising a bacteria consortium of anaerobic bacterial strains, in particular of anaerobic bacterial strains of an intestinal microbiome, wherein said consortium comprises i) Agathobacter rectalis and ii) Anaerostipes caccae and/or Anaerobutyricum hallii and comprises no more than 15 different bacterial strains, for use in the treatment of a disease or disorder caused by or related to an intestinal dysbiosis.

The invention particularly concerns a composition comprising a bacteria consortium of anaerobic bacterial strains, wherein said consortium comprises i) Agathobacter rectalis and ii) Anaerostipes caccae and/or Anaerobutyricum hallii and comprises no more than 15 different bacterial strains, for use in the treatment of an intestinal dysbiosis or of a disease or disorder caused by or related to an intestinal dysbiosis.

The intestinal dysbiosis is particularly selected from the group consisting of an intestinal dysbiosis following antibiotics treatment, an intestinal dysbiosis following infection by vancomycin resistant enterococci, an intestinal dysbiosis following infection by carbapenem resistant enterococci, and an intestinal dysbiosis following post-infectious diarrhea.

The disease or disorder caused by or related to an intestinal dysbiosis is particularly selected from the group consisting of inflammatory bowel disease, including ulcerative colitis and Crohn's disease; rheumatoid arthritis; multiple sclerosis; graft versus host disease; solid and liquid cancer, in particular gastrointestinal cancer, colorectal cancer and acute myeloid leukaemia.

In particular, the disease or disorder caused by or related to an intestinal dysbiosis is selected from the group consisting of dysbiosis following antibiotics treatment, following infection by vancomycin resistant enterococci, following infection by carbapenem resistant enterococci, or following post-infectious diarrhea; inflammatory bowel disease, including ulcerative colitis and Crohn's disease; rheumatoid arthritis; multiple sclerosis; graft versus host disease; solid and liquid cancers, in particular gastrointestinal cancer, colorectal cancer and acute myeloid leukemia.

In some embodiments, the consortium comprises i) Agathobacter rectalis and ii) Anaerostipes caccae and/or Anaerobutyricum hallii and iii) one or several bacterial strain(s) able to convert primary substrates into formate, lactate, succinate, acetate, butyrate and/or propionate, wherein the primary substrates are selected from the group consisting of sugars, starches, fibers and proteins and any combination thereof.

Preferably, the consortium comprises one or several bacterial strain(s) able to convert primary substrates into lactate.

Additionally or alternatively, the consortium comprises:
one or several bacterial strain(s) able to convert primary substrates into formate;
one or several bacterial strain(s) able to convert formate into acetate; and/or
one or several bacterial strain(s) able to convert primary substrates into acetate.

Additionally or alternatively, the consortium comprises:
one or several bacterial strain(s) able to convert primary substrates into succinate; and/or
one or several bacterial strain(s) able to convert succinate into propionate.

Preferably:
the strain able to convert primary substrates into formate is able to transform at least 20% of the total carbon transformed into the combined metabolites formate, lactate, succinate; acetate, propionate, butyrate and ethanol into formate when grown at least 48 hours in single culture on standard medium;
   the strain able to convert primary substrates into acetate is able to transform at least 30% of the total carbon transformed into the combined metabolites formate, lactate, succinate; acetate, propionate, butyrate and ethanol into acetate when grown at least for at least 48 hours in single culture on standard medium;
   the strain able to convert primary substrates into butyrate is able to transform at least 30% of the total carbon transformed into the combined metabolites formate, lactate, succinate; acetate, propionate, butyrate and ethanol into butyrate when grown for at least 48 hours in single culture on standard medium;
   the strain able to convert primary substrates into lactate is able to transform at least 30% of the total carbon transformed into the combined metabolites formate, lactate, succinate; acetate, propionate, butyrate and ethanol into lactate when grown for at least 48 hours in single culture on standard medium;
   the strain able to convert primary substrates into lactate is able to transform at least 30% of the total carbon transformed into the combined metabolites into propionate when grown for at least 48 hours in single culture on standard medium;
   the strain able to convert primary substrates into lactate is able to transform at least 30% of the total carbon transformed into the combined metabolites formate, lactate, succinate; acetate, propionate, butyrate and ethanol into propionate when grown for at least 48 hours in single culture on standard medium;
   the strain able to convert primary substrates into lactate is able to transform at least 30% of the total carbon transformed into the combined metabolites formate, lactate, succinate; acetate, propionate, butyrate and ethanol into succinate when grown for at least 48 hours in single culture on standard medium;
   the strain able to convert formate into acetate is able to degrade at least 20% of formate and transform at least 20% of the total carbon transformed into the combined metabolites formate, lactate, succinate; acetate, propionate, butyrate and ethanol into acetate when grown for at least 48 hours in single culture on standard medium comprising or supplemented with formate;
   the strain able to convert acetate into butyrate is able to degrade at least 20% of acetate and transform at least 20% of the total carbon transformed into the combined metabolites formate, lactate, succinate; acetate, propionate, butyrate and ethanol into butyrate when grown for at least 48 hours in single culture on standard medium comprising or supplemented with acetate;
   the strain able to convert lactate into butyrate is able to degrade at least 20% of lactate and transform at least 20% of the total carbon transformed into the combined metabolites formate, lactate, succinate; acetate, propionate, butyrate and ethanol into butyrate when grown for at least 48 hours in single culture on standard medium comprising or supplemented with lactate;
   the strain able to convert lactate into propionate is able to degrade at least 20% of lactate and transform at least 20% of the total carbon transformed into the combined metabolites formate, lactate, succinate; acetate, propionate, butyrate and ethanol into propionate when grown for at least 48 hours in single culture on standard medium comprising or supplemented with lactate; and/or
   the strain able to convert succinate into propionate is able to degrade at least 20% of formate and transform at least 20% of the total carbon transformed into the combined metabolites formate, lactate, succinate; acetate, propionate, butyrate and ethanol into propionate when grown for at least 48 hours in single culture on standard medium comprising or supplemented with succinate.

Preferably, the composition does not comprise one or more bacterial strain(s) selected from the species Anaerotignum nov sp., Clostridium_E sporosphaeroides, Coprococcus_A catus, Frisingococcus sp., Veillonella atypica, Veillonella parvula subsp. parvula and Veillonella ratti. More preferably, the composition does not comprise one or more bacterial strain(s) selected from the genera Anaerotignum, Clostridium_E, Coprococcus_A, Frisingococcus and Veillonella. Even more preferably, the composition does not comprise a bacterial strain able to convert lactate into propionate.

Preferably, in the consortium according to the invention:
the one or several bacterial strain(s) able to convert primary substrates into formate is/are selected from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus; and/or
the one or several bacterial strain(s) being able to convert primary substrates into acetate is/are selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia, and Rhiziobiaceae genus; more preferably from the genera Bifidobacterium, Blautia, Desulfovibrio, Dorea, Eubacterium, and Rhiziobiaceae genus, even more preferably from the genus Bifidobacterium; and/or
the one or several bacterial strain(s) being able to convert primary substrates into lactate is/are selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus, and Sutterella, even more preferably from the genus Lactobacillus; and/or
the one or several bacterial strain(s) being able to convert primary substrates into succinate is/are selected from the genera Acutalibacter nov genus, Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides or Prevotella; and/or
the one or several bacterial strain(s) being able to convert formate into acetate is/are selected from the genera Blautia, Eubacterium, Paraclostridium, Terrisporobacter and Intestinibacter; preferably Blautia and Eubacterium, even more preferably from the genus Blautia or Intestinibacter; and/or
the one or several bacterial strain(s) being able to convert succinate into propionate is/are selected from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

Preferably, in the consortium according to the invention:
the one or several bacterial strain(s) able to convert primary substrates into formate is/are selected from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas;
the one or several bacterial strain(s) being able to convert primary substrates into acetate is/are selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella;
the one or several bacterial strain(s) being able to convert primary substrates into lactate is/are selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella;
the one or several bacterial strain(s) being able to convert primary substrates into succinate is/are selected from the genera Acutalibacter novel genus, Bacteroides, Oliverbapstia, Parabacteroides, Phocaeicola and Prevotella;
the one or several bacterial strain(s) being able to convert formate into acetate is/are selected from the genera Blautia, Eubacterium; Terrisporobacter and Intestinibacter; and/or the one or several bacterial strain(s) being able to convert succinate into propionate is/are selected from the genera Dialister, Flavonifractor, Phascolarctobacterium Phascolarctobacterium_A and Veillonella. Preferably, in the consortium according to the invention:
the one or several bacterial strain(s) able to convert primary substrates into formate is/are selected from the genera Blautia, Coprococcus, Dorea, Erysipelatoclostridium, Faecalibacterium, Lachnospira and Ruminococcus;
the one or several bacterial strain(s) being able to convert primary substrates into acetate is/are selected from the genera Bifidobacterium, Blautia, Clostridium_Q, Clostridium_E, Desulfovibrio, Dorea, Eubacterium, Oliverpabstia and Rhiziobiaceae genus;
the one or several bacterial strain(s) being able to convert primary substrates into lactate is/are selected from the genera Agathobacter, Bacteroides, Bariatricus, Collinsella, Enterococcus, Lacticaseibacillus/Lactobacillus, Peptostreptococcus, Streptococcus, and Sutterella;
the one or several bacterial strain(s) being able to convert primary substrates into succinate is/are selected from the genera Acutalibacter nov genus, Bacteroides, Parabacteroides, Phocaeicola and Prevotella;
the one or several bacterial strain(s) being able to convert formate into acetate is/are selected from the genera Blautia, Eubacterium, Terrisporobacter and Intestinibacter; and/or the one or several bacterial strain(s) being able to convert succinate into propionate is/are selected from the genera Dialister, Flavonifractor, Phascolarctobacterium and Phascolarctobacterium_A.

Particularly, in the consortium according to the invention:
the one or several bacterial strain(s) able to convert primary substrates into formate is from the genus Ruminococcus;
the one or several bacterial strain(s) being able to convert primary substrates into acetate is from the genus Bifidobacterium;
the one or several bacterial strain(s) being able to convert primary substrates into lactate is from the genus Lactobacillus;
the one or several bacterial strain(s) being able to convert primary substrates into succinate is from the genus Bacteroides or Prevotella;
the one or several bacterial strain(s) being able to convert formate into acetate is from the genus Blautia or Terrisporobacter, preferably from the genus Blautia and/or
the one or several bacterial strain(s) being able to convert succinate into propionate is from the genus Phascolarctobacterium.

Even more preferably, in the consortium according to the invention:
the one or several bacterial strain(s) able to convert primary substrates into formate is/are selected from the species Bacteroides clarus (e.g. DSM 22519, JCM 16067), Blautia hydrogenotrophica (e.g. DSM 10507, JCM 14656), Collinsella aerofaciens (e.g. ATCC 25986, DSM 3979, JCM 10188), Coprococcus eutactus (e.g. ATCC 27759), Dorea formicigenerans (e.g. ATCC 27755, DSM 3992, JCM 31256), Dorea longicatena (e.g. DSM 13814, JCM 11232), Erysipelatoclostridium ramosum (e.g. ATCC 25582, DSM 1402, JCM 1298), Eubacterium ramulus (e.g. ATCC 29099, DSM 15684, JCM 31355), Faecalibacterium prausnitzii (e.g. DSM 17677, ATCC 27768, ATCC 27766, JCM 31915), Lachnospira (Eubacterium) eligens (e.g. ATCC 27750, DSM 3376), Longicatena sp. (in particular having a 16SRNA sequence such as set forth in SEQ ID NO: 11 or any variant thereof having at least 97%, 98% or 99% sequence identity thereto), Ruminococcus bromii (e.g. ATCC 27255) and Sellimonas intestinalis (e.g., JCM 30749); preferably from the species Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromil; most preferably is from the specie Ruminococcus bromii; and/or
the one or several bacterial strain(s) being able to convert primary substrates into acetate is/are selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g. ATCC 15703, DSM 20083, JCM 1251), Bifidobacterium catenulatum (e.g. ATCC 27539, DSM 16992, JCM 1194), Bifidobacterium longum (e.g. ATCC 15707, DSM 20219, JCM 1217), Bifidobacterium pseudocatenulatum (e.g. ATCC 27919, DSM 20438, JCM 1200), Bifidobacterium ruminantium (e.g. ATCC 49390, DSM 6489), Blautia hydrogenotrophica (e.g. DSM 10507, JCM 14656), Clostridium perfringens (e.g. ATCC 13124, DSM 756), Clostridium_E sporosphaeroides (e.g. ATCC 25781, DSM 1294), Clostridium_Q symbiosum (e.g. ATCC 14940, DSM 934, JCM 1297), Collinsella aerofaciens (e.g. ATCC 25986, DSM 3979, JCM 10188), Copromonas sp. (in particular having a 16SRNA sequence such as set forth in SEQ ID NO: 2, 3, 4 or 8, or any variant thereof having at least 97%, 98% or 99% sequence identity thereto), Desulfovibrio piger (e.g. ATCC 29098, DSM 749), Dorea formicigenerans (e.g., ATCC 27755, DSM 3992, JCM 31256), Dorea longicatena (e.g. DSM 13814, JCM 11232), Dorea scindens, Dorea sp. 900066555, Enterocloster sp. (in particular having a 16SRNA sequence such as set forth in SEQ ID NO: 12 or any variant thereof having at least 97%, 98% or 99% sequence identity thereto), Escherichia coli (e.g. ATCC 11775, DSM 30083, JCM 1649), Eubacterium callanderi (e.g. ATCC 49165, DSM 3662, JCM 10284), Eubacterium limosum (e.g. ATCC 8486, DSM 20543, JCM 6421, JCM 9978), Hungatella effluvii (e.g. DSM 24995), Hungatella_A sp. (in particular having a 16SRNA sequence such as set forth in SEQ ID NO: 5 or any variant thereof having at least 97%, 98% or 99% sequence identity thereto), Oliverpabstia sp. (in particular having a 16SRNA sequence such as set forth in SEQ ID NO: 7 or any variant thereof having at least 97%, 98% or 99% sequence identity thereto), Peptoniphilus vaginalis (e.g. DSM 101742), Peptostreptococcus anaerobius (e.g. ATCC 27337, DSM 2949), Phocaeicola plebeius (e.g. DSM 17135, JCM 12973), Rhizobiaceae nov. genus, (in particular having a 16SRNA sequence such as set forth in SEQ ID NO: 13 or any variant thereof having at least 97%, 98% or 99% sequence identity thereto), Ruminoccocus bromii (e.g. ATCC 27255), Sellimonas intestinalis (e.g. JCM 30749) and Veillonella ratti (e.g. ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. and Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus; most preferably is from the specie Bifidobacterium adolescentis, and/or
the one or several bacterial strain(s) being able to convert primary substrates into lactate is/are selected from the species Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 or any variant thereof having at least 97%, 98% or 99% sequence identity thereto), Merdisoma Aerosacheriphilus (in particular having a 16SRNA sequence such as set forth in SEQ ID NO: 10 or any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter faecis (also named Roseburia faecis), Bacteroides clarus, Bacteroides uniformis, Bariatricus comes, Collinsella aerofaciens, Enterococcus faecalis, Enterococcus faecium, Enterococcus gallinarum, (also named Lacticaseibacillus rhamnosus Lactobacillus rhamnosus), Peptostreptococcus stomatis, Streptococcus anginosus and Sutterella wadsworthensis; more preferably from the species Bacteroides uniformis, Bariatricus comes, Collinsella aerofaciens, Enterococcus faecalis, Enterococcus faecium, Enterococcus gallinarum, Lacticaseibacillus rhamnosus (also named Lactobacillus rhamnosus), Streptococcus anginosus and Sutterella wadsworthensis; most preferably is from the specie Lactobacillus rhamnosus and/or
the one or several bacterial strain(s) being able to convert primary substrates into succinate is/are selected from the species Acutalibacter nov. genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 or any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464); preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov. genus, Bacteroides faecis, Bacteroides fragilis, Bacteroides thetaiotaomicron, Bacteroides xylanisolvens, Phocaeicola plebeius, Phocaeicola vulgatus and Prevotella copri; most preferably from the species Acutalibacter nov. genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Prevotella copri and Bacteroides xylanisolvens,
the one or several bacterial strain(s) being able to convert formate into acetate is/are selected from the species Blautia hydrogenotrophica (e.g., DSM 10507, JCM 14656), Blautia_A wexlerae, Blautia_A luti (e.g., DSM 14534), Eubacterium callanderi (e.g., ATCC 49165, DSM 3662, JCM 10284) Eubacterium limosum (e.g., ATCC 8486, DSM 20543, JCM 6421, JCM 9978), Terrisporobacter glycolicus (e.g., DSM 1288, ATTC 14880), Terrisporobacter mayombei (e.g., DSM 6539), Terrisporobacter othiniensis (e.g., DSM 29186) and Intestinibacter bartlettii (e.g., DSM 16795), preferably from the species Blautia hydrogenotrophica or Intestinibacter bartlettii; and/or
the one or several bacterial strain(s) being able to convert succinate into propionate is/are selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512), preferably from Dialister hominis, Dialister invisus, Flavonifractor plautii, Phascolarctobacterium faecium and Phascolarctobacterium_A succinatutens; more preferably from the specie Phascolarctobacterium faecium.

In particular embodiments, in the consortium according to the invention:
the one or several bacterial strain(s) able to convert primary substrates into formate is/are selected from the species Bacteroides clarus, Blautia hydrogenotrophica, Collinsella aerofaciens, Coprococcus eutactus, Dorea formicigenerans, Dorea longicatena, Erysipelatoclostridium ramosum, Eubacterium ramulus, Faecalibacterium prausnitzii, Lachnospira eligens, Longicatena sp., Ruminococcus bromii and Sellimonas intestinalis;
the one or several bacterial strain(s) being able to convert primary substrates into acetate is/are selected from the species Acidaminococcus intestini, Acutalibacter nov. genus having a 16SRNA sequence as set forth in SEQ ID NO: 9 and any variant thereof having at least 97%, 98% or 99% sequence identity thereto, Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium perfringens, Clostridium_E sporosphaeroides, Clostridium_Q symbiosum, Collinsella aerofaciens, Copromonas sp, Desulfovibrio piger, Dorea formicigenerans, Dorea scindens, Dorea sp. 900066555, Enterocloster sp., Escherichia coli, Eubacterium callanderi, Eubacterium limosum, Hungatella effluvii, Hungatella_A sp., Oliverpabstia sp., Peptoniphilus vaginalis, Peptostreptococcus anaerobius, Phocaeicola plebeius, Rhizobiaceae nov. genus, Ruminoccocus bromii, Sellimonas intestinalis and Veillonella ratti;
the one or several bacterial strain(s) being able to convert primary substrates into lactate is/are selected from the species Agathobacter/Roseburia faecis, Bacteroides clarus, Bacteroides faecis, Bacteroides uniformis, Bariatricus comes, Bifidobacterium adolescentis, Collinsella aerofaciens, Enterococcus faecalis, Enterococcus faecium, Enterococcus gallinarum, Lacticaseibacillus rhamnosus/Lactobacillus rhamnosus, Longicatena sp., Merdisoma Aerosacheriphilus, Peptostreptococcus stomatis, Roseburia hominis, Streptococcus anginosus and Sutterella wadsworthensis;
the one or several bacterial strain(s) being able to convert primary substrates into succinate is/are selected from the species Acutalibacter nov. genus having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides faecis, Bacteroides fragilis, Bacteroides thetaiotaomicron, Bacteroides xylanisolvens, Oliverpabstia sp., Bacteroides faecis, Parabacteroides distasonis, Phocaeicola dorei, Phocaeicola plebeius, Phocaeicola vulgatus and Prevotella copri;
the one or several bacterial strain(s) being able to convert formate into acetate is/are selected from the species Blautia hydrogenotrophica, Blautia_A luti, Blautia_A wexlerae in particular having a 16SRNA sequence as set forth in SEQ ID NO: 17 or a variant of at least 90%, 95%, or 99% identity thereto, Terrisporobacter othiniensis, Terrisporobacter glycolicus, Terrisporobacter mayombei, Intestinibacter bartlettii, Eubacterium callanderi and Eubacterium limosum; and/or
the one or several bacterial strain(s) being able to convert succinate into propionate is/are selected from the species Dialister hominis, Dialister invisus, Flavonifractor plautii, Phascolarctobacterium faecium, Phascolarctobacterium_A succinatutens, Veillonella parvula subsp. Parvula and Veillonella ratti. In particular embodiments, in the consortium according to the invention:
the one or several bacterial strain(s) able to convert primary substrates into formate is/are selected from the species Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii;
the one or several bacterial strain(s) being able to convert primary substrates into acetate is/are selected from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp., Eubacterium callanderi, Eubacterium limosum, Oliverpabstia sp. And Rhizobiaceae nov. genus;
the one or several bacterial strain(s) being able to convert primary substrates into lactate is/are selected from the species Agathobacter faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus, Peptostreptococcus stomatis, Streptococcus anginosus and Sutterella wadsworthensis;
the one or several bacterial strain(s) being able to convert primary substrates into succinate is/are selected from the species Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri;
the one or several bacterial strain(s) being able to convert formate into acetate is/are selected from the species Blautia hydrogenotrophica, Blautia_A luti, Blautia_A wexlerae in particular having a 16SRNA sequence as set forth in SEQ ID NO: 17 or a variant of at least 90%, 95%, or 99% identity thereto, Terrisporobacter othiniensis, Terrisporobacter glycolicus, Terrisporobacter mayombei, Intestinibacter bartlettii, Eubacterium callanderi and Eubacterium limosum; and/or
the one or several bacterial strain(s) being able to convert succinate into propionate is/are selected from the species Dialister hominis, Dialister invisus, Flavonifractor plautii, Phascolarctobacterium faecium and Phascolarctobacterium_A succinatutens.

In particular embodiments, in the consortium according to the invention:
the one or several bacterial strain(s) able to convert primary substrates into formate is/are selected from the species Dorea formicigenerans, Dorea longicatena, Faecalibacterium prausnitzii and Ruminococcus bromii;
the one or several bacterial strain(s) being able to convert primary substrates into acetate is/are selected from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum and Rhizobiaceae nov. genus,
the one or several bacterial strain(s) being able to convert primary substrates into lactate is/are selected from the species Bacteroides uniformis, Bariatricus comes, Collinsella aerofaciens, Enterococcus faecalis, Enterococcus faecium, Enterococcus gallinarum, Lacticaseibacillus rhamnosus, Streptococcus anginosus and Sutterella wadsworthensis;
the one or several bacterial strain(s) being able to convert primary substrates into succinate is/are selected from the species Acutalibacter nov. genus, in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto, Prevotella copri and Bacteroides xylanisolvens;
the one or several bacterial strain(s) being able to convert formate into acetate is/are selected from the species Blautia hydrogenotrophica, Blautia_A luti, Blautia_A wexlerae in particular having a 16SRNA sequence as set forth in SEQ ID NO: 17 or a variant of at least 90%, 95%, or 99% identity thereto, Terrisporobacter othiniensis, Terrisporobacter glycolicus and Terrisporobacter mayombei; and/or the one or several bacterial strain(s) being able to convert succinate into propionate is/are selected from the species Dialister hominis, Dialister invisus, Flavonifractor plautii, Phascolarctobacterium faecium and Phascolarctobacterium_A succinatutens.

In particular embodiments, in the consortium according to the invention:
the one or several bacterial strain(s) able to convert primary substrates into formate is from the specie Ruminococcus bromii;
the one or several bacterial strain(s) being able to convert primary substrates into acetate is/are selected from the species Bifidobacterium adolescentis, Bifidobacterium catenulatum, Bifidobacterium longum, Bifidobacterium pseudocatenulatum and Bifidobacterium ruminantium the one or several bacterial strain(s) being able to convert primary substrates into lactate is from the species Lactobacillus rhamnosus;
the one or several bacterial strain(s) being able to convert primary substrates into succinate is/are selected from the species Acutalibacter nov. genus, Bacteroides faecis, Bacteroides fragilis, Bacteroides thetaiotaomicron, Bacteroides xylanisolvens, and Prevotella copri;
the one or several bacterial strain(s) being able to convert formate into acetate is/are selected from the species Blautia hydrogenotrophica, Blautia_A luti, Blautia_A wexlerae, Terrisporobacter othiniensis, Terrisporobacter glycolicus, Terrisporobacter mayombei, and/or
the one or several bacterial strain(s) being able to convert succinate into propionate is from the species Phascolarctobacterium faecium.

In particular embodiments, in the consortium according to the invention:
the one or several bacterial strain(s) able to convert primary substrates into formate is Ruminococcus bromii;
the one or several bacterial strain(s) being able to convert primary substrates into acetate Bifidobacterium adolescentis,
the one or several bacterial strain(s) being able to convert primary substrates into lactate is Lactobacillus rhamnosus;
the one or several bacterial strain(s) being able to convert primary substrates into succinate is/are selected from the species Acutalibacter nov. genus, in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto, Prevotella copri and Bacteroides xylanisolvens;
the one or several bacterial strain(s) being able to convert formate into acetate is Blautia hydrogenotrophica; and/or
the one or several bacterial strain(s) being able to convert succinate into propionate is Phascolarctobacterium faecium.

In particular embodiments, in the consortium according to the invention:
the one or several bacterial strain(s) able to convert primary substrates into formate comprise, or consist of, Ruminococcus bromii; and/or
the one or several bacterial strain(s) being able to convert primary substrates into acetate comprise, or consist of, Bifidobacterium adolescentis; and/or
the one or several bacterial strain(s) being able to convert primary substrates into lactate comprise, or consist of, Lacticaseibacillus rhamnosus (also named Lactobacillus rhamnosus); and/or
the one or several bacterial strain(s) being able to convert primary substrates into succinate is/are selected from Bacteroides xylanisolvens, Prevotella copri and Acutalibacter nov. genus having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto, preferably selected from Bacteroides xylanisolvens and Prevotella copri; and/or
the one or several bacterial strain(s) being able to convert formate into acetate comprise, or consist of, Blautia hydrogenotrophica; and/or
the one or several bacterial strain(s) being able to convert succinate into propionate comprise, or consist of, Phascolarctobacterium faecium.

In particular, the bacteria consortium of the invention comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and:
a bacterium selected from the group consisting of Bacteroides xylanisolvens, Prevotella copri and a bacterium Acutalibacter nov. genus having a DNA genome sequence having 90%, 95% or 99% sequence identity with SEQ ID NO: 1; and
a bacterium selected from the group consisting of Blautia hydrogenotrophica, Paraclostridium bifermentans, Terrisporobacter glycolicus, Terrisporobacter mayombei, Intestinibacter bartlettii, Eubacterium callanderi and Eubacterium limosum, preferably from the group consisting of Paraclostridium bifermentans, Terrisporobacter glycolicus, Terrisporobacter mayombei, Intestinibacter bartlettii, Eubacterium callanderi and Eubacterium limosum.

Alternatively, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and:
a bacterium selected from the group consisting of Bacteroides xylanisolvens, Prevotella copri and a bacterium Acutalibacter nov. genus having a DNA genome sequence having 90%, 95% or 99% sequence identity with SEQ ID NO: 1; and
a bacterium selected from the group consisting of Blautia hydrogenotrophica, Blautia_A luti, Blautia_A wexlerae, Terrisporobacter othiniensis, Terrisporobacter glycolicus, Terrisporobacter mayombei, Intestinibacter bartlettii, Eubacterium callanderi and Eubacterium limosum, preferably from the group consisting of Terrisporobacter glycolicus, Terrisporobacter othiniensis, Blautia_A luti and Blautia_A wexlerae in particular having a 16SRNA sequence as set forth in SEQ ID NO: 17 or a variant of at least 90%, 95%, or 99% identity thereto.

Preferably, the bacteria consortium of the invention comprises or essentially consists of:
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and Prevotella copri; or Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Blautia hydrogenotrophica; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Blautia hydrogenotrophica; Phascolarctobacterium faecium and Prevotella copri; or
Agathobacter rectalis; Anaerobutyricum hallii; Ruminococcus bromii; Bifidobacterium adolescentis; Lactobacillus rhamnosus; Blautia hydrogenotrophica; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Blautia hydrogenotrophica; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Blautia hydrogenotrophica; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Blautia hydrogenotrophica; Phascolarctobacterium faecium and a bacterium having a DNA genome sequence having at least 90%, preferably at least 95%, even more preferably at least 99% sequence identity with SEQ ID NO: 1.

In some aspects, Blautia hydrogenotrophica can be replaced by a bacterium selected from the group consisting of Paraclostridium bifermentans, Terrisporobacter glycolicus, Terrisporobacter mayombei, Intestinibacter bartlettii, Eubacterium callanderi and Eubacterium limosum, preferably by Intestinibacter bartlettii, in any of the bacteria consortium described above. Alternatively, Blautia hydrogenotrophica can be replaced by a bacterium selected from the group consisting of Blautia_A luti, Blautia_A wexlerae, Terrisporobacter othiniensis, Terrisporobacter glycolicus, Terrisporobacter mayombei, Intestinibacter bartlettii, Eubacterium callanderi and Eubacterium limosum, preferably from the group consisting of Blautia_A luti, Blautia_A wexlerae, Terrisporobacter othiniensis, Terrisporobacter glycolicus, Terrisporobacter mayombei, even more preferably from the group consisting of Terrisporobacter othiniensis, Blautia_A luti, Blautia_A wexlerae, in any one of the bacteria consortium described herein.

In some aspects, the bacteria consortium of the invention comprises or consists of:
Agathobacter rectalis; Anaerostipes caccae; Ruminococcus bromii; Bifidobacterium adolescentis; Lactobacillus rhamnosus; Blautia hydrogenotrophica; Phascolarctobacterium faecium and Bacteroides xylanisolvens;
Agathobacter rectalis; Anaerostipes caccae; Ruminococcus bromii; Bifidobacterium adolescentis; Lactobacillus rhamnosus; Blautia hydrogenotrophica; Phascolarctobacterium faecium and Prevotella copri;
Agathobacter rectalis; Anaerostipes caccae; Ruminococcus bromii; Bifidobacterium adolescentis; Lactobacillus rhamnosus; Blautia hydrogenotrophica; Phascolarctobacterium faecium and a bacterium having a DNA genome sequence of SEQ ID NO: 1;
Agathobacter rectalis; Anaerostipes caccae; Ruminococcus bromii; Bifidobacterium adolescentis; Lactobacillus rhamnosus; Terrisporobacter othiniensis, Phascolarctobacterium faecium and Bacteroides xylanisolvens; or
Agathobacter rectalis; Anaerostipes caccae; Ruminococcus bromii; Bifidobacterium adolescentis; Lactobacillus rhamnosus; Blautia_A wexlerae, Phascolarctobacterium faecium and Bacteroides xylanisolvens.

In particular, the composition of the invention comprises between 5 and 15 different bacterial strains, preferably between 6 and 10 different bacterial strains.

Preferably, the composition of the invention is free of, or essentially free of succinate, formate and/or lactate.

In some embodiments, the composition of the invention comprises:
propionate, acetate and/or butyrate;
a pharmaceutical excipient or carrier; and/or
a component selected from the group consisting of cryoprotecting media, in particular glycerol; culture or dispersing media, in particular comprising peptone, yeast extract, monosaccharides, disaccharides, arabinogalactan, fructo-oligosaccharides, fibers, glycerol, soluble starch, resistant starch, xylan, minerals, co-factors, vitamins and reducing agents; aqueous gels; prebiotics and polymeric supports; and any combination thereof.

In other aspects, the invention concerns a composition as defined herein and a bioreactor comprising such composition.

In other aspects, the invention concerns a method for treating an intestinal dysbiosis or a disease or disorder caused by or related to an intestinal dysbiosis in a subject, wherein the method comprises administering a composition of the invention to said subject.

The invention finally concerns the use of a composition of the invention for the manufacture of a medicament for the treatment of an intestinal dysbiosis or of a disease or disorder caused by or related to an intestinal dysbiosis.

### DETAILED DESCRIPTION OF THE INVENTION

### Definitions

Unless otherwise defined, all terms of art, notations and other scientific terminology used herein are intended to have the meanings commonly understood by those of skill in the art to which this invention pertains.

In order that the present invention may be more readily understood, certain terms are defined hereafter. Additional definitions are set forth throughout the detailed description.

As used herein, the terms “microbiome” and “microbiota” are equivalent and refer to the ecological community of commensal, symbiotic, and pathogenic microorganisms that literally share the same given habitat or host. These terms particularly refer to the human gut or intestinal microbiome.

The terms “bacterium”, “bacterium strain” and “bacterial strain” can be used interchangeably and denote any bacterium of the taxonomic domain Bacteria. Due to their functions, species of the genera Methanobrevibacter and Candidatus Methanomassiliicoccus (also named herein Methanomassiliicoccus) belonging to the taxonomic domain Archaea shall be herein included in these terms. Preferably, terms “bacterium”, “bacterium strain” and “bacterial strain” refer to the taxonomic domain Bacteria.

Definitions of species used herein are defined by sequence identity of their whole genome and/or characteristic sections thereof (as the case may be via reference to a public database). For example, bacteria with a 16S rRNA sequence similarity of at least 95%, 97%, 98%, 99%, 99.5%, 99.6%, 99.7%, 99.8% or 99.9%, preferably 99.9%, are considered as belonging to a same taxon. As the skilled person is aware, nomenclature is constantly subject to changes, due to developing methods of analysis and approaches of the research community. For instance, the genus of Clostridia has been sub divided into Clostridum_E, Clostridium_Q, etc. Previous and current assignments of genomes to taxons can be retrieved, for example, from https://www.ncbi.nlm.nih.gov/Taxonomy/Browser/wwwtax.cgi or from https://https://gtdb.ecogenomic.org/. Changes in the nomenclature which have been suggested by the research community or a part of the research community and may be of relevance to the present invention include reclassification of Clostridium sporosphaeroides to Clostridium_E sporosphaeroides, Clostridium symbiosum to Clostridium_Q symbiosum, Coprococcus catus to Coprococcus_A catus, Hungatella sp. To Hungatella_A species, Phascolarctobacterium succinatutens to Phascolarctobacterium_A succinatutens, Ruminococcus bromii to Ruminococcus_E bromii. Whenever the traditional term for a genus is used (e.g. Clostridium), it is also meant to cover newly introduced related genera (e.g. Clostridium_E, Clostridium_Q, etc.)

It should be noticed that bacterium Clostridium lactatifermentans has been recently renamed Anaerotignum lactatifermentans. Then, as used herein, the terms “Clostridium lactatifermentans” and “Anaerotignum lactatifermentans” have the same meaning and can be used interchangeably.

It should also be noticed that bacterium “Eubacterium rectale” has been renamed “Agathobacter rectalis”. Then, as used herein, the terms “Eubacterium rectale” and “Agathobacter rectalis” have the same meaning and can be used interchangeably.

It should be noted that bacterium “Eubacterium hallii” has been renamed “Anaerobutyricum hallii”. Then, as used herein, the terms “Eubacterium hallii” and “Anaerobutyricum hallii” have the same meaning and can be used interchangeably.

It should be noted that bacterium “Agathobacter faecis” has been renamed “Roseburia faecis”. Then, as used herein, the terms “Agathobacter faecis” and “Roseburia faecis” have the same meaning and can be used interchangeably.

It should be noted that bacterium “Lactobacillus rhamnosus” has been renamed “Lacticaseibacillus rhamnosus”. Then, as used herein, the terms “Lactobacillus rhamnosus” and “Lacticaseibacillus rhamnosus” have the same meaning and can be used interchangeably.

It should be noted that bacterium “Eubacterium eligens” has been renamed “Lachnospira eligens”. Then, as used herein, the terms “Eubacterium eligens” and “Lachnospira eligens” have the same meaning and can be used interchangeably.

It should be noted that “Clostridium bartlettii” has been renamed “Intestinibacter bartlettii”. Then, as used herein, the terms “Clostridium bartlettii” and “Intestinibacter bartlettii” have the same meaning and can be used interchangeably.

It should be noted that “Clostridium glycolicum” has been renamed “Terrisprobacter glycolicus”. Then, as used herein, the terms “Clostridium glycolicum” and “Terrisporobacter glycolicus” have the same meaning and can be used interchangeably.

It should be noted that “Clostridium mayombei” has been renamed “Terrisporobacter mayombei”. Then, as used herein, the terms “Clostridium mayombei” and “Terrisporobacter mayombei” have the same meaning and can be used interchangeably.

It should be noted that “Clostridium bifermentans” has been renamed “Paraclostridium bifermentans”. Then, as used herein, the terms “Clostridium bifermentans” and “Paraclostridium bifermentans” have the same meaning and can be used interchangeably.

The term “consortium”, “microbial consortium” or “bacterial consortium” refers herein to at least three microbial organisms, preferably of five or more bacteria, officiating in the same metabolic or trophic network. Preferably, microbial members of the consortium collaborate, in particular for their subsistence, into the consortium. More preferably, each bacterium of the consortium (i) produces a compound which is utilized by another bacterium of the consortium and/or (ii) utilizes a compound which is produced by another bacterium of the consortium.

The term “short chain fatty acids” (SCFA) is also known as volatile fatty acids (VFAs) and specifically denotes fatty acids with two to six carbon atoms.

As used herein, the term “intermediate metabolite” denotes a metabolite produced by bacteria that are used as energy source or substrates by other bacteria. Such intermediate metabolites in particular may include degradation products from fibers, proteins or other organic compounds. Preferably, the intermediate metabolites are one or more of formate, lactate and succinate. More generally, the term “intermediate metabolites” may refer to an undesirable metabolite, the presence or amount of which being limited as much as possible in the composition according to the invention.

As used herein, the term “end metabolites” denotes the metabolites produced by bacteria that are not or only partially utilized by other bacteria. Preferably, end metabolites include butyrate and acetate and/or propionate, more preferably butyrate. More generally, the term “end metabolites” may refer to a desirable metabolite, the presence or amount of which being enriched in the composition according to the invention.

As used herein, the term “a”, “an”, “the” and similar terms used in the context of the present invention (especially in the context of the claims) are to be construed to cover both the singular and plural unless otherwise indicated herein or clearly contradicted by the context.

As used herein, the terms “including”, “containing” and “comprising” are used herein in their open, non-limiting sense.

As used herein, the term “consist essentially of” refers to those elements required for a given embodiment. This term indicates the inclusion of any recited characteristics and permits the optional presence of elements that do not materially affect nor change the characteristics or functions of said embodiment. Preferably, in the context of a composition comprising bacteria, it refers to a composition that comprises the recited bacteria, and optionally includes other components such as prebiotics, at the exclusion of other microorganisms such as bacteria. In preferred embodiments, the term “consist essentially of” refers a composition that comprises the recited bacteria, and optionally includes other components such as prebiotics, at the exclusion of other bacteria.

The term “at least one” means “one or more” or “one or several”. For instance, it refers to one, two, three or more.

### Consortium of Bacterial Strains

The inventors have developed specific consortia of bacterial species which both reflect the intestinal microbiome complexity and increase desirable end metabolites production, especially butyrate. The bacterial strains of the consortia disclosed herein have been selected to enable metabolic cross-feeding interactions or collaboration between each other.

Preferably, bacteria of the consortia disclosed herein are bacteria of the intestinal or gut microbiota, in particular of human gut or intestinal microbiota. Preferably, such bacteria are not pathogenic bacteria. This means that bacteria according to the invention are known as not able to trigger any disease or disorder in a subject. Preferably, bacteria used in the present invention are bacteria strains of class I.

Preferably, the bacteria of the consortia used in the present invention are facultatively or strictly anaerobic.

Preferably, the bacteria of the consortium are isolated or purified bacteria, such as isolated or purified from a human intestinal microbiome. In microbiology, the term “isolation” or “purification” refers to the separation of a bacterial strain from a natural, mixed population of living microbes, such as the gut microbiota, or extracted from a sample of such gut microbiota, such as a fecal or stool sample.

Preferably, the bacteria of the consortia disclosed herein are live bacteria. The terms “viable bacterium” and “live bacterium” can be used interchangeably and denote a bacterium which has the capacity to grow under suitable conditions. Bacterial viability can be measured using biochemical assays. Preferably, these terms relate to a bacterium strain (i) having a viability of over 50% (e.g., in compositions such as pharmaceutical compositions), typically over 60% and preferably over 90%, in particular as determined by flow cytometry.

Preferably, the bacteria of the consortia disclosed herein are able to convert substrates and/or metabolites. As used herein, the term “convert” or “conversion” refers to the transformation of a substrate into a metabolite or to the transformation of a metabolite into another, particularly through enzymatic reactions, such as within metabolic pathways.

Techniques for measuring substrates and/or metabolites conversion are well known in the art, such as metabolomics, isotope labelling and metabolic flux analysis to trace the conversion and flow of metabolites, Pulsed stable isotope-resolved metabolomics, nuclear magnetic resonance (NMR) spectroscopy, Mass spectrometry-based metabolomics (such as LC-MS, GC-MS, HPLC-MS), or biological assays. These analytical techniques enable the detection, identification, and quantification of metabolites and their conversion products,

In order to create a consortium reflecting the gut microbiota complexity, the inventors have identified key functions/pathways that are performed by bacteria of an intestinal microbiome. When bacteria are assembled into a particular consortium according to the invention, each of the bacterial strain of the consortium is able to perform a pathway selected from the group consisting of pathways A1, A2, A3, A4, A5, A6, B1, B2, B3, B5, C1 and C2 and any combination thereof. Optionally, a bacterial strain of the consortium may be able to perform pathway B4. Preferably, the consortium does not comprise any bacteria which is able to perform pathway B4. These pathways are defined below.

As used herein, pathway (A1) corresponds to the conversion of primary substrates and production of formate. Then, a bacterial strain able to convert primary substrates such as sugars, starches, fibers and/or proteins into formate performs pathway A1.

In particular, bacteria performing pathway (A1) are able to transform at least 20%, at least 25%, more preferably at least 30% of the total carbon transformed into the combined metabolites, i.e. formate (FO), lactate (LT), succinate (SU), acetate (AA), propionate (PA), butyrate (BA) and Ethanol (Et), into formate, especially when grown for at least 48 hours in single culture on standard medium. In other words, after the experiment, at least 20%, at least 25% or at least 30%, preferably at least 30%, of the total carbon in the combined metabolites (FO+LT+SU+AA+PA+BA+Et) is present as formate.

By “single culture” it is meant a method of growing isolated bacteria from a single specie or genus on a nutrient standard medium under controlled condition.

By “standard medium” is meant a media that will support the growth of a wide variety of bacteria. Standard medium usually comprises a carbon source, a source of potassium and/or phosphorus; a source of nitrogen and/or sulfur; and a source of magnesium.

Examples of suitable standard medium are YCFA and M2GSC which are well known to those of skill in the art. YCFA can be prepared as described in Duncan et al., Roseburia intestinalis sp. Nov., a novel saccharolytic, butyrate-producing bacterium from human faeces. Int J Syst Evol Microbiol. 2002; 52:1615-1620. Doi: 10.1099/00207713-52-5-1615. It is mainly composed of casitone, yeast extract, buffer (NaHCO₃), reducing agent (cysteine, resazurin), mineral sources (K2HPO4, KH2PO4, NaCl, MgSO4, CaCl2)), vitamins (biotin, cobalamin, aminobenzoic acid, folic acid, pyridoxamine). M2GSC can be prepared as described in Miyazaki et al., Degradation and utilization of xylans by the rumen anaerobe Prevotella bryantii (formerly P. ruminicola subsp. Brevis) B14. Anaerobe. 1997; 3:373-381. Doi: 10.1006/anae.1997.0125. It is mainly composed of casitone, yeast extract, buffer (NaHCO₃), glucose, cellbiose, soluble starch, rumen fluid, reducing agent (cysteine, resazurin), mineral sources (K2HPO4, KH2PO4, NaCl, MgSO4, CaCl2)).

As used herein, pathway (A2) corresponds to the conversion of primary substrates and production of acetate. Then, a bacterial strain able to convert primary substrates such as sugars, starches, fibers and/or proteins into acetate performs pathway A2.

In particular, bacteria performing pathway (A2) are able to transform at least 30%, at least 50%, more preferably at least 60% of the total carbon transformed into the combined metabolites FO, LT, SU, AA, PA, BA and Ethanol into acetate, especially when grown for at least 48 hours in single culture on standard medium. In other words, after the experiment, at least 30%, at least 50% or at least 60%, preferably 60%, of the total carbon in the combined metabolites (FO+LT+SU+AA+PA+BA+Et) is present as acetate.

As used herein, pathway (A3) corresponds to the conversion of primary substrates and production of butyrate.

In particular, bacteria performing pathway (A3) are able to transform at least 30%, at least 50%, more preferably at least 60% of the total carbon transformed into the combined metabolites FO, LT, SU, AA, PA, BA and Ethanol into butyrate, especially when grown for at least 48 hours in single culture on standard medium. In other words, after the experiment, at least 30%, preferably at least 50%, more preferably 60%, of the total carbon in the combined metabolites (FO+LT+SU+AA+PA+BA+Et) is present as butyrate.

As used herein, pathway (A4) corresponds to the conversion of primary substrates and production of lactate. In particular, bacteria performing pathway (A4) are able to transform at least 30%, at least 50%, more preferably at least 60% of the total carbon transformed into FO, LT, SU, AA, PA, BA and Ethanol into lactate, especially when grown for at least 48 hours in single culture on standard medium. In other words, after the experiment, at least 30%, preferably at least 50%, more preferably at least 60% of the total carbon in the combined metabolites (FO+LT+SU+AA+PA+BA+Et) is present as lactate.

As used herein, pathway (A5) corresponds to the conversion of primary substrates and production of propionate.

In particular, bacteria performing pathway (A5) are able to transform at least 30%, at least 50%, more preferably at least 60% of the total carbon transformed into FO, LT, SU, AA, PA, BA and Ethanol into acetate, especially when grown for at least 48 hours in single culture on standard medium. In other words, after the experiment, at least 30%, preferably at least 50%, more preferably at least 60% of the total carbon in the combined metabolites (FO+LT+SU+AA+PA+BA+Et) is present as acetate.

As used herein, pathway (A6) corresponds to the conversion of primary substrates and production of succinate.

In particular, bacteria performing pathway (A6) are able to transform at least 30%, at least 50%, more preferably at least 60% of the total carbon transformed into FO, LT, SU, AA, PA, BA and Ethanol into succinate, especially when grown for at least 48 hours in single culture on standard medium. In other words, after the experiment, at least 30%, preferably at least 50%, more preferably at least 60% of the total carbon in the combined metabolites (FO+LT+SU+AA+PA+BA+Et) is present as succinate.

As used herein, pathway (B1) corresponds to the conversion of formate and production of acetate.

In particular, bacteria performing pathway (B1) are able to degrade at least 20% of formate in medium and transform at least 20%, preferably at least 50%, more preferably at least 65% of the total carbon transformed into the combined metabolites FO, LT, SU, AA, PA, BA and Ethanol into acetate, especially when grown for at least 48 hours in single culture on standard medium comprising or supplemented with formate. In other words, after the experiment, at least 30%, preferably at least 50%, more preferably at least 65% of the total carbon in the combined positively accumulated metabolites (FO+LT+SU+AA+PA+BA+Et|Δ_{metabolite}>0) is present as acetate.

As used herein, pathway (B2) corresponds to the conversion of acetate and production of butyrate.

In particular, bacteria performing pathway (B2) degrade at least 20% of acetate in medium and transform at least 20%, preferably at least 50%, more preferably at least 65% of the total carbon transformed into the combined metabolites FO, LT, SU, AA, PA, BA and Ethanol into butyrate, especially when grown for at least 48 hours in single culture on standard medium or standard medium comprising or supplemented with FO, LT and/or SU. In other words, after the experiment, at least 20%, preferably at least 50%, more preferably at least 65% of the total carbon in the combined positively accumulated metabolites (FO+LT+SU+AA+PA+BA+Et|Δ_{metabolite}>0) is present as butyrate.

As used herein, pathway (B3) corresponds to the conversion of lactate and production of butyrate.

In particular, bacteria performing pathway (B3) degrade at least 20% of lactate in medium and transform at least 20%, preferably at least 50%, more preferably at least 65% of the total carbon transformed into the combined metabolites FO, LT, SU, AA, PA, BA and Ethanol into butyrate, especially when grown for at least 48 hours in single culture on standard medium comprising or supplemented with lactate. In other words, after the experiment, at least 20%, preferably at least 50%, more preferably at least 65% of the total carbon in the combined positively accumulated metabolites (FO+LT+SU+AA+PA+BA+Et|Δ_{metabolite}>0) is present as butyrate. As used herein, pathway (B4) corresponds to the conversion of lactate and production of propionate.

In particular, bacteria performing pathway (B4) degrade at least 20% of lactate in medium and transform at least 20%, preferably at least 50%, more preferably at least 65% of the total carbon transformed into the combined metabolites FO, LT, SU, AA, PA, BA and Ethanol into propionate, especially when grown for at least 48 hours in single culture on standard medium comprising or supplemented with lactate. In other words, after the experiment, at least 20%, preferably at least 50%, more preferably at least 65% of the total carbon in the combined positively accumulated metabolites (FO+LT+SU+AA+PA+BA+Et|Δ_{metabolite}>0) is present as propionate.

As used herein, pathway (B5) corresponds to the conversion of succinate and production of propionate.

In particular, bacteria performing pathway (B5) degrade at least 20% of succinate in medium and transform at least 20%, preferably at least 50%, more preferably at least 65% of the total carbon transformed into the combined metabolites FO, LT, SU, AA, PA, BA and Ethanol into propionate, especially when grown for at least 48 hours in single culture on standard medium comprising or supplemented with succinate. In other words, after the experiment, at least 20%, preferably at least 50%, more preferably at least 65% of the total carbon in the combined positively accumulated metabolites (FO+LT+SU+AA+PA+BA+Et|Δ_{metabolite}>0) is present as propionate.

As used herein, pathway (C1) corresponds to the conversion of primary substrates to lactate while reducing oxygen.

As used herein, pathway (C2) corresponds to the conversion of H2, CO2 and formate to acetate.

Literature data on hydrogen utilization or oxygen tolerability were used to assign C-reactions to the strains, whereby oxygen reduction was assigned to strains reported to grow under aerobic or microaerophilic conditions and hydrogen consumption to acetogenic or methanogenic gut bacteria. Bacterial strains being able to maintain concentrations in the culture medium of 02 below a concentration inhibiting proliferation of at least one bacterial strain of the consortium can be identified as described in literature (02 concentration <2 mg/L in the liquid phase and/or RedOx potential <−250 mV; see Ricciardi et al., Rapid detection assay for oxygen consumption in the Lactobacillus casei group, Ann Microbiol 64, 1861-1864 (2014). Bacterial strains being able to maintain concentrations in the culture medium of H2 below a concentration inhibiting proliferation of at least one bacterial strain of the consortium can be identified as described in literature, by analyzing the H2 amount in the headspace of a Hungate tube (preferably below 4%; see Leclerc et al., H2/CO2 metabolism in acetogenic bacteria, isolated from the human colon, Anaerobe, 3 (5), 307-315 (1997); Pham et al., Lactate-utilizing community is associated with gut microbiota dysbiosis in colicky infants. Sci Rep.; 7 (1): 11176 (2017)).

Then, bacteria are particularly defined herein by their capacities or abilities to perform particular pathways of an intestinal microbiome. Such abilities are for example capacity to degrade or convert a particular substrate, for example such as starch, and to produce a particular product or metabolite, for example such as butyrate. Generally, one bacterium is able to degrade or convert a substrate (e.g., starch) and to produce a product (e.g., butyrate). Accordingly, bacteria that are able to degrade or convert the same substrate(s) (e.g., starch) and to produce the same metabolite(s) (e.g., butyrate) performed the same pathways.

Such capacities of a bacterium are well known in the art. Experiments are known in the art to test whether a bacterial strain is able to perform a metabolic pathway. For example, the degradation of sugars, starches or fibers can be tested simply by providing such substrate to bacteria while observing or monitoring their growth. For example, bacteria can be characterized for growth and metabolite production on M2GSC Medium (e.g., ATCC Medium 2857) and modifications thereof whereby the carbon sources glucose, cellobiose and starch are replaced by specific substrates including intermediate metabolites and/or fibers, preferably such as found in the human intestine. The concentrations of the produced metabolites can for example be quantified by any analytic method available for the person skilled in the art such as refractive index detection high pressure liquid chromatography (HPLC-RI; for example, as provided by Thermo Scientific Accela™). Additional examples of such experiments are provided below for each of the different groups of pathways. In such examples, the concentration of metabolites is preferably determined by centrifuging a liquid culture sample before/after the at least 48 hours growth period, filtering the supernatant, and subjecting filtrate to refractive index detection high pressure liquid chromatography (HPLC-RI).

In particular, to test whether a bacterial strain is able to perform pathways (A1)-(A6), the experiment can be as follows: the bacterial strain, when grown in single culture, strictly anaerobically, in Hungate tubes for at least 48 hours on YCFA medium (Yeast extract, casitone, fatty acids) and/or M2GSC medium (Glucose, soluble starch, cellbiose medium) at pH 6.5-7 and 37° C., is able to transform a defined minimal share of the total carbon converted into the combined metabolites FO, LT, SU, AA, PA, BA and Et into the metabolite of interest. For example, the condition for a bacterial strain to qualify as a formate producer (performing pathway A1) is:

$$\frac{( Δ FO [ mM ] ) * 1}{( \begin{matrix}Δ FO [ mM ] * 1 + Δ AA [ mM ] * 2 + Δ BA [ mM ] * 4 + Δ LT [ mM ] * 3 + \\ Δ PA [ mM ] * 3 + Δ SU [ mM ] * 4 + Δ ethanol [ mM ] * 2\end{matrix} )} > 0.2$$

For instance, the minimal share for formate (pathway A1) is 0.2, preferably 0.25, more preferably 0.3. The minimal share for acetate (A2), butyrate (A3), lactate (A4), propionate (A5) and succinate (A6) are 0.3, preferably 0.5, more preferably 0.6.

To test if a bacterial strain is able to perform pathways (B1), (B3)-(B5) the experiment can be as follows: the bacterial strain, when grown in single culture, strictly anaerobically, in Hungate tubes for at least 48 hours on modified M2GSC or YCFA medium at pH 6.5-7 and 37° C., degrade at least 20% of the intermediate metabolite of interest (FO, LT, SU), and transform at least 20%, preferably at least 50%, more preferably at least 65% of the total carbon converted into the combined metabolites FO, LT, SU, AA, PA, BA and Et (i.e., those of FO, LT, SU, AA, PA, BA and Et which are positively accumulated) into the respective end metabolite of interest (AA, BA, PA). The modified media M2FO, M2LT and M2SU particularly contain 30 mM of formate (FO), 30 mM of DL-lactate (LT), or 30 mM of sodium succinate (SU), respectively.

For example, the conditions for a bacterial strain to qualify as a lactate to butyrate converter (performing pathway B3), are:

$$\begin{matrix}1 ) & \\ \frac{Δ LA [ mM ]}{LA initial [ mM ]} < - 0.2 & \end{matrix}$$

$$\begin{matrix}2 ) & \\ \frac{( Δ BA [ mM ] ) * 4}{sum ( \begin{matrix}\begin{matrix}Δ FO [ mM ] * 1 + Δ AA [ mM ] * 2 + Δ BA [ mM ] * \\ 4 + Δ LT [ mM ] * 3 + Δ PA [ mM ] * 3 +\end{matrix} \\ Δ SU [ mM ] * 4 + Δ ethanol [ mM ] * 2 ‖ Δ metabolite [ mM ] > 0\end{matrix} )} > 0.2 & \end{matrix}$$

Finally, to test if a bacterial strain is able to perform pathway (B2) the experiment can be as follows: the bacterial strain, when grown in single culture, strictly anaerobically, in Hungate tubes for at least 48 hours on YCFA, M2GSC medium or their modified form as described above, at pH 6.5-7 and 37° C., degrade at least 20% of acetate in medium, and transform at least 20%, preferably at least 50%, more preferably at least 65% of the total carbon converted into the combined metabolites FO, LT, SU, AA, PA, BA and Et (i.e. those of FO, LT, SU, AA, PA, BA and Et which are positively accumulated) into butyrate.

For example, the conditions for a bacterial strain to qualify as an acetate to butyrate converter (performing pathway B2) are:

$$\begin{matrix}1 ) & \\ \frac{Δ AA [ mM ]}{AA initial [ mM ]} < - 0.2 & \end{matrix}$$

$$\begin{matrix}2 ) & \\ \frac{( Δ AA [ mM ] ) * 4}{sum ( \begin{matrix}\begin{matrix}Δ FO [ mM ] * 1 + Δ AA [ mM ] * 2 + Δ BA [ mM ] * \\ 4 + Δ LT [ mM ] * 3 + Δ PA [ mM ] * 3 +\end{matrix} \\ Δ SU [ mM ] * 4 + Δ ethanol [ mM ] * 2 ‖ Δ metabolite [ mM ] > 0\end{matrix} )} > 0.2 & \end{matrix}$$

A single strain can be tested for each of the above conditions to determine whether it is able to perform one or more metabolic pathway(s) such as described herein.

Preferably, Agathobacter rectalis bacteria comprised in the consortium according to the invention are able to perform pathways A4 and/or B2, preferably pathways A4 and B2 such as described above.

Preferably, Anaerostipes caccae bacteria comprised in the consortium according to the invention are able to perform pathways A3 and/or B2 and/or B3, preferably pathways A3, B2 and B3 such as described above.

Preferably, Anaerobutyricum hallii bacteria comprised in the consortium according to the invention are able to perform pathways A3 and/or B2 and/or B3 and optionally C2, preferably pathways A3, B2 and B3 such as described above.

The invention concerns consortium of anaerobic bacterial strains of an intestinal microbiome, wherein said consortium comprises of i) Agathobacter rectalis, and ii) Anaerostipes caccae and/or Anaerobutyricum hallii and no more than 15 different bacterial strains.

In some embodiments, the bacterial ratio between i) Agathobacter rectalis, and ii) Anaerostipes caccae and/or Anaerobutyricum hallii is comprised between 10⁷:1 and 1:10⁷, preferably between 10⁵:1 and 1:10⁵, more preferably between 10³:1 and 1:10³, particularly preferably between 100:1 and 1:100, most preferably between 10:1 and 1:10.

In some embodiments, the bacterial ratio between Agathobacter rectalis and Anaerostipes caccae is comprised between 10⁷:1 and 1:10⁷, preferably between 10⁵:1 and 1:10⁵, more preferably between 10³:1 and 1:10³, particularly preferably between 100:1 and 1:100, most preferably between 10:1 and 1:10.

In some embodiments, the bacterial ratio between Agathobacter rectalis and Anaerobutyricum hallii is comprised between 10⁷:1 and 1:10⁷, preferably between 10⁵:1 and 1:10⁵, more preferably between 10³:1 and 1:10³, particularly preferably between 100:1 and 1:100, most preferably between 10:1 and 1:10.

As used herein the term “bacterial ratio” refers to the ratio of the number of bacterial cells, amount or abundance of the bacterial strains, in particular in a culture medium. It is typically calculated by dividing the amount of i) Agathobacter rectalis, and ii) Anaerostipes caccae and/or Anaerobutyricum hallii.

Preferably, the abundance or amount of a bacterial strain is measured using optical density, qPCR, microarray technique, amplicon sequencing, single cell sequencing or single cell Matrix Assisted Laser Desorption Ionization-Time of Flight (MALDI-TOF) following flow cytometry or other microfluidic methods, chamber counting, total bacterial DNA quantification or metagenomic sequencing and grouping of genes, proteomic profiling or total RNA sequencing of complex samples.

In some embodiments, the consortium according to the invention comprises no more than 15, 14, 13, 12, 11, 10, 9, or 8 different bacterial strains. Particularly, the consortium according to the invention comprises 2, 3, 4, 5, 6, 7, 8, 9, 10, 11, 12, 13, 14, or 15 different bacterial strains, preferably 5, 6, 7, 8, 9, 10, 11, 12, 13, 14, or 15 different bacterial strains, more preferably 8, 9, 10, 11, 12, 13, 14, or 15 different bacterial strains, and even more preferably 8 different bacterial strains. In particular, the consortium according to the invention may comprise between 5 and 15 different bacterial strains, preferably between 5 and 10 different bacterial strains, more preferably between 6 and 10 different bacterial strains, and even more preferably between 7 and 9 different bacterial strains.

Preferably, the consortium comprises or consists of i) Agathobacter rectalis and ii) Anaerostipes caccae and/or Anaerobutyricum hallii and iii) one or several additional bacterial strain(s) but no more than 13, 12, 11, 10, 9, 8, 7, 6 different bacterial strains.

In some embodiment, the consortium comprises i) Agathobacter rectalis and ii) Anaerostipes caccae and/or Anaerobutyricum hallii and iii) one or several bacterial strain(s) able to convert primary substrates into formate, lactate, succinate, acetate, butyrate and/or propionate, wherein the primary substrates are selected from the group consisting of sugars, starches, fibers and proteins and any combination thereof.

In some embodiments, the consortium comprises one or more bacterial strains that is able to perform one or more pathways such as described above, in particular selected from the group consisting of pathways A1, A2, A3, A4, A5, A6, B1, B2, B3, B4, B5, C1 and C2, and combinations thereof. Preferably, the consortium comprises or consists of i) Agathobacter rectalis, and ii) Anaerostipes caccae and/or Anaerobutyricum hallii and iii) one or several bacterial strain(s) able to perform one or more pathways selected from the group consisting of pathway A1, A2, A3, A4, A5, A6, B1, B2, B3, B4, B5, C1 and C2, preferably A1, A2, A4, A5, A6, B1, B5, C1 and C2, even more preferably A1, A2, A4, A6, B1 and B5.

Preferably, the invention concerns a consortium of no more than 15 different anaerobic bacterial strains of an intestinal microbiome, wherein said consortium comprises or consists of i) Agathobacter rectalis and ii) Anaerostipes caccae and/or Anaerobutyricum hallii and iii) between 1 and 13, preferably between 5 and 10, even more preferably between 7 and 9, different bacterial strains able to perform at least one pathway selected from the group consisting of pathway A1, A2, A3, A4, A5, A6, B1, B2, B3, B4, B5, C1 and C2, preferably A1, A2, A4, A5, A6, B1, B5, C1 and C2, even more preferably A1, A2, A4, A6, B1 and B5.

In a specific embodiment, for each pathway A1, A2, A3, A4, A5, A6, B1, B2, B3, B4, B5, preferably for each pathway A1, A2, A4, A6, B1, B2, B3 and B5, one or more bacterial representatives (i.e., one or more bacterium that is representative of the specie or genus), preferably one bacterial representative, is comprised in a consortium. Also, some of the represented pathways may be accomplished by one or more than one bacterial strain. In other words, one bacterium can be able to perform a plurality of pathways and/or one pathway may be performed by one or more bacterial strains present in the consortium.

In an embodiment, the consortium according to the invention comprises or consists of i) Agathobacter rectalis (performing pathway B2 and A4), and ii) Anaerostipes caccae and/or Anaerobutyricum hallii (performing pathways B2, B3, A3) and iii):
one or several bacterial strain(s) able to convert primary substrates such as sugars, starches, fibers and/or proteins into formate, (pathway A1)
one or several bacterial strain(s) able to convert primary substrates such as sugars, starches, fibers and/or proteins into lactate into acetate (pathway A2);
optionally one or several bacterial strain(s) able to convert primary substrates into butyrate (pathway A3);
one or several bacterial strain(s) able to convert primary substrates such as sugars, starches, fibers and/or proteins into lactate optionally while reducing oxygen (pathway A4 and optionally pathway C1);
optionally one or several bacterial strain(s) able to convert primary substrates into propionate (pathway A5);
one or several bacterial strain(s) able to convert primary substrates such as sugars, starches, fibers and/or proteins into lactate into succinate (pathway A6);
one or several bacterial strain(s) able to convert formate into acetate preferably able to convert of H2, CO2 and formate to acetate (pathway B1, preferably pathway C2);
optionally one or several bacterial strain(s) able to convert acetate into butyrate (pathway B2);
optionally one or several bacterial strain(s) able to convert lactate into butyrate (pathway B3)
optionally, one or several bacterial strain(s) able to convert lactate into propionate (pathway B4);
and/or
one or several bacterial strain(s) able to convert succinate into propionate (pathway B5).

In an embodiment, the consortium according to the invention comprises or consists of i) Agathobacter rectalis and ii) Anaerostipes caccae and/or Anaerobutyricum hallii and iii):
one or several bacterial strain(s) able to convert primary substrates such as sugars, starches, fibers and/or proteins into formate, (pathway A1)
one or several bacterial strain(s) able to convert primary substrates such as sugars, starches, fibers and/or proteins into lactate into acetate (pathway A2);
one or several bacterial strain(s) able to convert primary substrates such as sugars, starches, fibers and/or proteins into lactate optionally while reducing oxygen (pathway A4 and optionally pathway C1);
optionally one or several bacterial strain(s) able to convert primary substrates into propionate (pathway A5);
one or several bacterial strain(s) able to convert primary substrates such as sugars, starches, fibers and/or proteins into lactate into succinate (pathway A6);
one or several bacterial strain(s) able to convert formate into acetate preferably able to convert of H2, CO2 and formate to acetate (pathway B1, preferably pathway C2);
optionally, one or several bacterial strain(s) able to convert lactate into propionate (pathway B4); and
one or several bacterial strain(s) able to convert succinate into propionate (pathway B5).

Preferably, said consortium does not comprise a bacterial strain able to convert lactate into propionate (pathway B4) and/or a bacterial strain able to convert primary substrates into propionate (pathway A5).

Said consortium may further comprise one or several additional bacterial strains able to perform one or more pathway(s) selected from the group consisting of A3, A4, B2, B3 and any combination thereof.

In some embodiments, the consortium according to the invention comprises or consists of i) Agathobacter rectalis and ii) Anaerostipes caccae and/or Anaerobutyricum hallii and iii) one or several bacterial strain(s) able to convert primary substrates into lactate.

In some embodiments, the consortium according to the invention comprises or consists of i) Agathobacter rectalis and ii) Anaerostipes caccae and/or Anaerobutyricum hallii and iii):
one or several bacterial strain(s) able to convert primary substrates into formate;
one or several bacterial strain(s) able to convert formate into acetate; and/or
one or several bacterial strain(s) able to convert primary substrates into acetate.

Additionally or alternatively, the consortium according to the invention comprises or consists of i) Agathobacter rectalis and ii) Anaerostipes caccae and/or Anaerobutyricum hallii and iii):
one or several bacterial strain(s) able to convert primary substrates into succinate; and/or
one or several bacterial strain(s) able to convert succinate into propionate.

Additionally or alternatively, the consortium according to the invention comprises or consists of i) Agathobacter rectalis and ii) Anaerostipes caccae and/or Anaerobutyricum hallii and iii):
one or several bacterial strain(s) able to convert primary substrates into propionate;
one or several bacterial strain(s) able to convert primary substrates into succinate; and/or
one or several bacterial strain(s) able to convert succinate into propionate.

In some embodiments, the consortium according to the invention comprises or consists of i) Agathobacter rectalis and ii) Anaerostipes caccae and/or Anaerobutyricum hallii and iii):
one or several bacterial strain(s) able to convert primary substrates such as sugars, starches, fibers and/or proteins into formate, (pathway A1);
one or several bacterial strain(s) able to convert primary substrates such as sugars, starches, fibers and/or proteins into lactate into acetate (pathway A2);
one or several bacterial strain(s) able to convert primary substrates such as sugars, starches, fibers and/or proteins into lactate optionally while reducing oxygen (pathway A4 and optionally pathway C1);
optionally one or several bacterial strain(s) able to convert primary substrates into propionate (pathway A5);
one or several bacterial strain(s) able to convert primary substrates such as sugars, starches, fibers and/or proteins into lactate into succinate (pathway A6);
one or several bacterial strain(s) able to convert formate into acetate preferably able to convert of H2, CO2 and formate to acetate (pathway B1, preferably pathway C2); and
one or several bacterial strain(s) able to convert succinate into propionate (pathway B5).

Said consortium may further comprise one or several additional bacterial strains able to perform pathways one or more pathway(s) selected from the group consisting of A3, A4, B2, B3 and any combination thereof.

Preferably, said consortium does not comprise a bacterial strain able to convert lactate into propionate (pathway B4) and/or a bacterial strain able to convert primary substrates into propionate (pathway A5).

As mentioned above, bacteria can be classified by their capacities to perform metabolic pathways such as disclosed herein. In addition, below are provided examples of bacteria genera and species that are able to perform the different metabolic pathways. Then, the man skilled in the art is able to select bacteria genera or species to fulfill the conditions of the different consortiums disclosed herein.

Whenever a strain is described by taxon or by reference to a public collection of microorganisms, what is claimed is the strain deposited under the accession number (e.g., ATCC, DSMZ, etc.) or strains which are closely related to the deposited strain, i.e., have the same or very similar physiological and biochemical characteristics. It is a common assumption that data obtained with one strain of a certain taxon is representative of other strains of the same taxon. For example, bacteria with a 16S rRNA sequence similarity of at least 95%, 97%, 98%, 99%, 99.5%, 99.6%, 99.7%, 99.8% or 99.9%, preferably 99.9%, are considered as belonging to the same taxon. Whenever a plurality of strains are described or claimed based on their affiliation with a genus/classification under a genus-level, the genus is defined as all strains having a 16S rRNA sequence similarity of at least 90%, 93%, 94% or 95%, preferably 95%, with a specifically listed representative of the said genus.

The bacterial strain(s) being able to perform pathway (A1) may be selected from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas and any combination thereof; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus.

In particular, the bacterial strain(s) being able to perform pathway (A1) may be selected from the species Anaerobutyricum/Eubacterium hallii (e.g. ATCC 27751, DSM 3353, JCM 31263), Bacteroides clarus (e.g. DSM 22519, JCM 16067), Blautia hydrogenotrophica (e.g. DSM 10507, JCM 14656), Collinsella aerofaciens (e.g. ATCC 25986, DSM 3979, JCM 10188), Coprococcus eutactus (e.g. ATCC 27759), Dorea formicigenerans (e.g. ATCC 27755, DSM 3992, JCM 31256), Dorea longicatena (e.g. DSM 13814, JCM 11232), Erysipelatoclostridium ramosum (e.g. ATCC 25582, DSM 1402, JCM 1298), Eubacterium ramulus (e.g. ATCC 29099, DSM 15684, JCM 31355), Faecalibacterium prausnitzii (e.g. DSM 17677, ATCC 27768, ATCC 27766, JCM 31915), Lachnospira (Eubacterium) eligens (e.g. ATCC 27750, DSM 3376), Longicatena sp. (in particular having a 16SRNA sequence such as set forth in SEQ ID NO: 11 or any variant thereof having at least 97%, 98% or 99% sequence identity thereto), Ruminococcus bromii (e.g. ATCC 27255) and Sellimonas intestinalis (e.g., JCM 30749) and any combination thereof; preferably from the species Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii; most preferably is from the specie Ruminococcus bromii.

The bacterial strain(s) being able to perform pathway (A2) may be selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella and any combination thereof; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Blautia and Bifidobaterium.

In particular, the bacterial strain(s) being able to perform pathway (A2) may be selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g. ATCC 15703, DSM 20083, JCM 1251), Bifidobacterium catenulatum (e.g. ATCC 27539, DSM 16992, JCM 1194), Bifidobacterium longum (e.g. ATCC 15707, DSM 20219, JCM 1217), Bifidobacterium pseudocatenulatum (e.g. ATCC 27919, DSM 20438, JCM 1200), Bifidobacterium ruminantium (e.g. ATCC 49390, DSM 6489), Blautia hydrogenotrophica (e.g. DSM 10507, JCM 14656), Clostridium perfringens (e.g. ATCC 13124, DSM 756), Clostridium_E sporosphaeroides (e.g. ATCC 25781, DSM 1294), Clostridium_Q symbiosum (e.g. ATCC 14940, DSM 934, JCM 1297), Collinsella aerofaciens (e.g. ATCC 25986, DSM 3979, JCM 10188), Copromonas sp. (in particular having a 16SRNA sequence such as set forth in SEQ ID NO: 2, 3, 4 or 8, or any variant thereof having at least 97%, 98% or 99% sequence identity thereto), Desulfovibrio piger (e.g. ATCC 29098, DSM 749), Dorea formicigenerans (e.g., ATCC 27755, DSM 3992, JCM 31256), Dorea longicatena (e.g. DSM 13814, JCM 11232), Dorea scindens, Dorea sp. 900066555, Enterocloster sp. (in particular having a 16SRNA sequence such as set forth in SEQ ID NO: 12 or any variant thereof having at least 97%, 98% or 99% sequence identity thereto), Escherichia coli (e.g. ATCC 11775, DSM 30083, JCM 1649), Eubacterium callanderi (e.g. ATCC 49165, DSM 3662, JCM 10284), Eubacterium limosum (e.g. ATCC 8486, DSM 20543, JCM 6421, JCM 9978), Hungatella effluvii (e.g. DSM 24995), Hungatella_A sp. (in particular having a 16SRNA sequence such as set forth in SEQ ID NO: 5 or any variant thereof having at least 97%, 98% or 99% sequence identity thereto), Oliverpabstia sp. (in particular having a 16SRNA sequence such as set forth in SEQ ID NO: 7 or any variant thereof having at least 97%, 98% or 99% sequence identity thereto), Peptoniphilus vaginalis (e.g. DSM 101742), Peptostreptococcus anaerobius (e.g. ATCC 27337, DSM 2949), Phocaeicola plebeius (e.g. DSM 17135, JCM 12973), Rhizobiaceae nov. genus, (in particular having a 16SRNA sequence such as set forth in SEQ ID NO: 13 or any variant thereof having at least 97%, 98% or 99% sequence identity thereto), Ruminoccocus bromii (e.g. ATCC 27255), Sellimonas intestinalis (e.g. JCM 30749) and Veillonella ratti (e.g. ATCC 17746, DSM 20736, JCM 6512) and any combination thereof; preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus; most preferably is from the specie Bifidobacterium adolescentis or Blautia hydrogenotrophica, preferably Bifidobacterium adolescentis.

The bacterial strain(s) being able to perform pathway (A3) may be selected from the genera Acidaminococcus, Agathobacter, Anaerobutyricum, Anaerostipes, Anaerotignum, Bariatricus, Clostridium, Clostridium_E, Clostridium_Q, Coprococcus, Coprococcus_A, Copromonas, Eubacterium, Faecalibacterium, Flavonifractor, Longicatena, Peptoniphilus and Roseburia and any combination thereof; preferably from the genera Acidaminococcus, Anaerobutyricum, Anaerostipes, Coprococcus, Coprococcus_A, Copromonas, Eubacterium, Faecalibacterium, Flavonifractor, Longicatena and Roseburia; more preferably from the genera Acidaminococcus, Anaerobutyricum, Anaerostipes, Copromonas, Eubacterium, Faecalibacterium, Flavonifractor, Longicatena and Roseburia, even more preferably from the genera Anaerobutyricum and Anaerostipes.

In particular, the bacterial strain(s) being able to perform pathway (A3) may be selected from the species Acidaminococcus intestini (e.g., DSM 21505, CIP 108586), Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp000436415, Bariatricus comes (e.g., ATCC 27758), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Coprococcus eutactus (e.g., ATCC 27759), Coprococcus_A catus (e.g., ATCC 27761), Copromonas sp. (in particular having a 16SRNA sequence such as set forth in SEQ ID NO: 2, 3, 4 or 8, and any variant thereof having at least 97%, 98% or 99% sequence identity thereto), Eubacterium ramulus (e.g., ATCC 29099, DSM 15684, JCM 31355), Faecalibacterium prausnitzii (e.g., DSM 17677, ATCC 27768, ATCC 27766, JCM 31915), Flavonifractor plautii (e.g., ATCC 29863, DSM 4000), Longicatena sp. (in particular having a 16SRNA sequence such as set forth in SEQ ID NO: 11 and any variant thereof having at least 97%, 98% or 99% sequence identity thereto), Peptoniphilus vaginalis (e.g., DSM 101742), Roseburia hominis (e.g., DSM 16839) and Roseburia intestinalis (e.g., DSM 14610, JCM 31262) and any combination thereof; preferably from the species Acidaminococcus intestini, Anaerobutyricum/Eubacterium hallii, Anaerostipes caccae, Anaerostipes hadrus, Coprococcus eutactus, Coprococcus_A catus, Copromonas sp., Eubacterium ramulus, Faecalibacterium prausnitzii, Flavonifractor plautii, Longicatena sp., Roseburia hominis and Roseburia intestinalis; more preferably from the species Acidaminococcus intestini, Anaerobutyricum/Eubacterium hallii, Anaerostipes caccae, Anaerostipes hadrus, Eubacterium ramulus, Faecalibacterium prausnitzii, Flavonifractor plautii, Roseburia hominis and Roseburia intestinalis.

The bacterial strain(s) being able to perform pathway (A4) may be selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella and any combination thereof; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably is from the genus Lactobacillus or Agathobacter.

In particular, the bacterial strain(s) being able to perform pathway (A4) may be selected from the species Agathobacter rectalis (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 or any variant thereof having at least 97%, 98% or 99% sequence identity thereto), Merdisoma Aerosacheriphilus (in particular having a 16SRNA sequence such as set forth in SEQ ID NO: 10 or any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016) and any combination thereof; preferably from the species Agathobacter rectalis (also named Eubacterium rectale), Agathobacter faecis (also named Roseburia faecis), Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus (also named Lactobacillus rhamnosus), Peptostreptococcus stomatis, Streptococcus anginosus and Sutterella wadsworthensis; more preferably from the species Agathobacter rectalis (also named Eubacterium rectale), Bacteroides uniformis, Bariatricus comes, Collinsella aerofaciens, Enterococcus faecalis, Enterococcus faecium, Enterococcus gallinarum, Lacticaseibacillus rhamnosus (also named Lactobacillus rhamnosus), Streptococcus anginosus and Sutterella wadsworthensis; even more preferably from the specie Lactobacillus rhamnosus, Agathobacter rectalis and Bifidobacterium adolescentis, most preferably is from the specie Lactobacillus rhamnosus.

The bacterial strain(s) being able to perform pathway (A5) may be selected from the genera Anaerotignum, Bacteroides, Phocaeicola and Veillonella and any combination thereof; preferably the strains are from the genus Bacteroides or Phocaeicola.

In particular, the bacterial strain(s) being able to perform pathway (A5) may be selected from the species Anaerotignum lactatifermentans (e.g., DSM 14214), Anaerotignum sp000436415, Bacteroides fragilis (e.g., ATCC 25285, DSM 2151, JCM 11019), Phocaeicola vulgatus (e.g., ATCC 8482, DSM 1447, JCM 5826) and Veillonella ratti (e.g., ATCC 17746, DSM 20736, JCM 6512) and any combination thereof; preferably from Bacteroides fragilis and Phocaeicola vulgatus.

The bacterial strain(s) being able to perform pathway (A6) may be selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella and any combination thereof; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Acutalibacter nov genus, Bacteroides and Prevotella, most preferably from the genera Bacteroides and Prevotella.

In particular, the bacterial strain(s) being able to perform pathway (A6) may be selected from the species Acutalibacter nov. genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 or any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464) and any combination thereof; preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov. genus, Bacteroides faecis, Bacteroides fragilis, Bacteroides thetaiotaomicron, Bacteroides xylanisolvens, Phocaeicola plebeius, Phocaeicola vulgatus and Prevotella copri; most preferably from the species Acutalibacter nov. genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Prevotella copri and Bacteroides xylanisolvens.

The bacterial strain(s) being able to perform pathway (B1) may be selected from the genera Blautia, Eubacterium, Paraclostridium, Terrisporobacter and Intestinibacter; preferably from the genus Blautia or Intestinibacter. Preferably, the bacterial strain(s) being able to perform pathway (B1) may be selected from the genera Eubacterium, Paraclostridium, Terrisporobacter and Intestinibacter, preferably Intestinibacter. Preferably, the bacterial strain(s) being able to perform pathway (B1) may be selected from the genera Blautia, Eubacterium, Terrisporobacter and Intestinibacter, preferably is from the genera Blautia or Terrisporobacter. In particular, the bacterial strain(s) being able to perform pathway (B1) may be selected from the genera Blautia and Eubacterium; preferably from the genus Blautia.

In particular, the bacterial strain(s) being able to perform pathway (B1) may be selected from the species Blautia hydrogenotrophica (e.g., DSM 10507, JCM 14656), Terrisporobacter othiniensis (e.g., DSM 29186), Blautia_A luti (e.g., DSM 14534), Blautia_A wexlerae (in particular having a 16SRNA sequence as set forth in SEQ ID NO: 17 and any variant thereof having at least 97%, 98% or 99% sequence identity thereto), Eubacterium callanderi (e.g., ATCC 49165, DSM 3662, JCM 10284), Eubacterium limosum (e.g., ATCC 8486, DSM 20543, JCM 6421, JCM 9978), Paraclostridium bifermentans (e.g., DSM 14991, JCM 1386), Terrisporobacter glycolicus (e.g., DSM 1288, ATCC 14880), Terrisporobacter mayombei (e.g., DSM 6539), Intestinibacter bartlettii (e.g., DSM 16795), preferably from the species Blautia hydrogenotrophica (e.g., DSM 10507, JCM 14656), Terrisporobacter othiniensis (e.g., DSM 29186), Blautia_A luti (e.g., DSM 14534), Eubacterium callanderi (e.g., ATCC 49165, DSM 3662, JCM 10284), Blautia_A wexlerae (in particular having a 16SRNA sequence as set forth in SEQ ID NO: 17 and any variant thereof having at least 97%, 98% or 99% sequence identity thereto), Eubacterium callanderi (e.g., ATCC 49165, DSM 3662, JCM 10284), Eubacterium limosum (e.g., ATCC 8486, DSM 20543, JCM 6421, JCM 9978), Terrisporobacter glycolicus (e.g., DSM 1288, ATCC 14880), Terrisporobacter mayombei (e.g., DSM 6539), Intestinibacter bartlettii (e.g., DSM 16795), and any combination thereof.

Preferably, the bacterial strain(s) being able to perform pathway (B1) may be selected from the species Blautia hydrogenotrophica (e.g., DSM 10507, JCM 14656), Eubacterium callanderi (e.g., ATCC 49165, DSM 3662, JCM 10284), Eubacterium limosum (e.g., ATCC 8486, DSM 20543, JCM 6421, JCM 9978), Paraclostridium bifermentans (e.g., DSM 14991, JCM 1386), Terrisporobacter glycolicus (e.g., DSM 1288, ATCC 14880), Terrisporobacter mayombei (e.g., DSM 6539), Intestinibacter bartlettii (e.g., DSM 16795) and any combination thereof, preferably is from the specie Blautia hydrogenotrophica or Intestinibacter bartlettii. Alternatively, the bacterial strain(s) being able to perform pathway (B1) may be selected from the species Blautia hydrogenotrophica (e.g., DSM 10507, JCM 14656), Eubacterium callanderi (e.g., ATCC 49165, DSM 3662, JCM 10284), Eubacterium limosum (e.g., ATCC 8486, DSM 20543, JCM 6421, JCM 9978), Terrisporobacter glycolicus (e.g., DSM 1288, ATCC 14880), Terrisporobacter mayombei (e.g., DSM 6539), Intestinibacter bartlettii (e.g., DSM 16795) and any combination thereof, preferably is from the specie Blautia hydrogenotrophica or Intestinibacter bartlettii. In particular, the bacterial strain(s) being able to perform pathway (B1) may be selected from the species Eubacterium callanderi (e.g., ATCC 49165, DSM 3662, JCM 10284), Eubacterium limosum (e.g., ATCC 8486, DSM 20543, JCM 6421, JCM 9978), Paraclostridium bifermentans (e.g., DSM 14991, JCM 1386), Terrisporobacter glycolicus (e.g., DSM 1288, ATCC 14880), Terrisporobacter mayombei (e.g., DSM 6539), Intestinibacter bartlettii (e.g., DSM 16795) and any combination thereof, preferably is from the specie Intestinibacter bartlettii. Particularly, the bacterial strain(s) being able to perform pathway (B1) may be selected from the species Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978) and any combination thereof, preferably from the specie Blautia hydrogenotrophica. Alternatively, the bacterial strain(s) being able to perform pathway (B1) may be selected from the species Blautia hydrogenotrophica (e.g., DSM 10507, JCM 14656), Terrisporobacter othiniensis (e.g., DSM 29186), Blautia_A luti (e.g., DSM 14534), Blautia_A wexlerae (in particular having a 16SRNA sequence as set forth in SEQ ID NO: 17 and any variant thereof having at least 97%, 98% or 99% sequence identity thereto), and any combination thereof, preferably is from the specie Blautia hydrogenotrophica.

The bacterial strain(s) being able to perform pathway (B2) may be selected from the genera Agathobacter, Anaerobutyricum, Anaerostipes, Anaerotignum, Desulfovibrio, Dysosmobacter, Faecalibacterium, Longicatena and Roseburia and any combination thereof; preferably from the genera Agathobacter, Anaerobutyricum, Anaerostipes, Desulfovibrio, Faecalibacterium and Roseburia.

In particular, the bacterial strain(s) being able to perform pathway (B2) may be selected from the species species Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp. (in particular having a 16RNA sequence as set forth in SEQ ID NO: 16 or a variant having at least 95%, preferably at least 97%, more preferably 99%, 99.5%, 99.7% or 99.9% sequence identity thereto), Desulfovibrio piger (e.g., ATCC 29098, DSM 749), Dysosmobacter sp. (in particular having a 16SRNA sequence such as set forth in SEQ ID NO: 6 and any variant thereof having at least 97%, 98% or 99% sequence identity thereto), Faecalibacterium prausnitzii (e.g., ATCC 27768, ATCC 27766, DSM 17677, JCM 31915), Longicatena sp., Roseburia hominis (e.g., DSM 16839) and Roseburia intestinalis (e.g., DSM 14610, JCM 31262) and any combination thereof; preferably Agathobacter rectalis, Anaerobutyricum hallii, Anaerostipes caccae, Anaerostipes hadrus, Desulfovibirio piger, Faecalibacterium prausnitzii, Roseburia hominis and Roseburia intestinalis; more preferably Anaerobutyricum hallii, Anaerostipes caccae and Agathobacter rectalis.

The bacterial strain(s) being able to perform pathway (B3) may be selected from the genera Anaerobutyricum, Anaerostipes and Eubacterium and any combination thereof; preferably from the genera Anaerobutyricum and Anaerostipes.

In particular, the bacterial strain(s) being able to perform pathway (B3) may be selected from the species Anaerobutyricum/Eubacterium hallii (e.g., ATCC 27751, DSM 3353, JCM 31263), Anaerostipes caccae (e.g., DSM 14662, JCM 13470), Eubacterium callanderi (e.g., ATCC 49165, DSM 3662, JCM 10284) and Eubacterium limosum (e.g., ATCC 8486, DSM 20543, JCM 6421, JCM 9978) and any combination thereof; preferably from Anaerobutyricum hallii and Anaerostipes caccae.

The bacterial strain(s) being able to perform pathway (B4) may be selected from the genera Anaerotignum, Clostridium (Clostridium_E), Coprococcus_A, Frisingococcus and Veillonella and any combination thereof; preferably from the genera Anaerotignum genus, Coprococcus_A, Frisingococcus and Veillonella.

In particular, the bacterial strain(s) being able to perform pathway (B4) may be selected from the species species Anaerotignum nov sp. (in particular having a 16SRNA sequence such as set forth in SEQ ID NO: 14 and any variant thereof having at least 97%, 98% or 99% sequence identity thereto), Clostridium_E sporosphaeroides (e.g., ATCC 25781, DSM 1294), Coprococcus_A catus (e.g., ATCC 27761), Frisingococcus sp. (in particular having a 16SRNA sequence such as set forth in SEQ ID NO: 15 and any variant thereof having at least 97%, 98% or 99% sequence identity thereto), Veillonella atypica (e.g., ATCC 17744, DSM 20739), Veillonella parvula subsp. Parvula (e.g., DSM 2008), Veillonella ratti (e.g., ATCC 17746, DSM 20736, JCM 6512) and any combination thereof, preferably from Anaerotignum nov sp., Coprococcus_A catus, Frisingococcus sp., Veillonella atypica and Veillonella parvula subsp. Parvula.

The bacterial strain(s) being able to perform pathway (B5) may be selected from the genera Dialister, Flavonifractor, Phascolarctobacterium, Phascolarctobacterium_A and Veillonella and any combination thereof; preferably from the genera Dialister, I Flavonifractor and Phascolarctobacterium and Phascolarctobacterium_A, more preferably from the genus Phascolarctobacterium.

In particular, the bacterial strain(s) being able to perform pathway (B5) may be selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512) and any combination thereof, preferably from Dialister hominis, Dialister invisus, Flavonifractor plautii, Phascolarctobacterium faecium and Phascolarctobacterium_A succinatutens; more preferably from the specie Phascolarctobacterium faecium.

The bacterial strain(s) s being able to perform pathway (C1) may be selected from the genera Enterococcus, Escherichia, Lacticaseibacillus/Lactobacillus, Lactococcus and Streptococcus and any combination thereof; preferably from the genera Enterococcus, Escherichia and Lacticaseibacillus.

In particular, the bacterial strain(s) being able to perform pathway (C1) may be selected from the species Enterococcus caccae (e.g., ATCC BAA-1240, DSM 19114), Enterococcus faecalis (e.g., ATCC 29212, DSM 2570), Escherichia coli (e.g., ATCC 11775, DSM 30083, JCM 1649), Lacticaseibacillus rhamnosus/Lactobacillus rhamnosus (e.g., ATCC 7469, DSM 20021, JCM1136), Lactococcus lactis (e.g., ATCC 19435, DSM 20481) and Streptococcus salivarius (e.g., ATCC 7073, DSM 20560, JCM 5707) and any combination thereof; preferably from Enterococcus faecalis, Escherichia coli and Lacticaseibacillus rhamnosus.

The bacterial strain(s) s being able to perform pathway (C2) may be selected from the genera Acetobacterium, Anaerobutyricum, Blautia, Candidatus Methanomassiliicoccus, Clostridium, Eubacterium Methanobrevibacter, Paraclostridium, Terrisporobacter, Intestinibacter and any combination thereof; preferably selected from the genera Intestinibacter, Blautia and Eubacterium. The bacterial strain(s) s being able to perform pathway (C2) may also be selected from the genera Acetobacterium, Anaerobutyricum, Candidatus Methanomassiliicoccus, Clostridium, Eubacterium Methanobrevibacter, Paraclostridium, Terrisporobacter, Intestinibacter and any combination thereof; preferably selected from the genera Intestinibacter and Eubacterium. The bacterial strain(s) s being able to perform pathway (C2) may particularly be selected from the genera Acetobacterium, Anaerobutyricum, Blautia, Candidatus Methanomassiliicoccus, Clostridium, Eubacterium and Methanobrevibacter and any combination thereof; preferably selected from the genera Anaerobutyricum, Blautia and Eubacterium.

In particular, the bacterial strain(s) being able to perform pathway (C2) may be selected from the species Acetobacterium carbinolicum (e.g., ATCC BAA-990, DSM 2925), Acetobacterium malicum (e.g., DSM 4132), Acetobacterium wieringae (e.g., ATCC 43740, DSM1911, JCM 2380), Blautia hydrogenotrophica (e.g., DSM 10507, JCM 14656), Blautia producta (e.g., ATCC 27340, DSM 2950, JCM 1471), Candidatus Methanomassiliicoccus intestinalis, Clostridium aceticum (e.g., ATCC 35044, DSM 1496, JCM 15732), Clostridium glycolicum (or Terrisporobacter glycolicus, e.g., ATCC 14880, DSM 1288, JCM 1401), Clostridium magnum (e.g., ATCC 49199, DSM 2767), Clostridium mayombe (or Terrisporobacter mayombei, e.g., ATCC 51428, DSM 2767, DSM 6539), Anaerobutyricum/Eubacterium hallii (e.g., ATCC 27751, DSM 3353, JCM 31263), Eubacterium limosum (e.g., ATCC 8486, DSM 20543, JCM 6421), Eubacterium ramulus (e.g., ATCC 29099, DSM 15684, JCM) and Methanobrevibacter smithii (e.g., ATCC 35061, DSM 861, JCM 328), Intestinibacter bartlettii (e.g., DSM 16795), Paraclostridium bifermentans (e.g., DSM 14991, JCM 1386) and any combination thereof; preferably selected from the species Intestinibacter bartlettii, Blautia hydrogenotrophica, Blautia producta, Eubacterium limosum and Eubacterium ramulus. For instance, the bacterial strain(s) being able to perform pathway (C2) may be selected from the species Acetobacterium carbinolicum (e.g., ATCC BAA-990, DSM 2925), Acetobacterium malicum (e.g., DSM 4132), Acetobacterium wieringae (e.g., ATCC 43740, DSM1911, JCM 2380), Candidatus Methanomassiliicoccus intestinalis, Clostridium aceticum (e.g., ATCC 35044, DSM 1496, JCM 15732), Clostridium glycolicum (or Terrisporobacter glycolicus, e.g., ATCC 14880, DSM 1288, JCM 1401), Clostridium magnum (e.g., ATCC 49199, DSM 2767), Clostridium mayombe (or Terrisporobacter mayombei, e.g., ATCC 51428, DSM 2767, DSM 6539), Anaerobutyricum/Eubacterium hallii (e.g., ATCC 27751, DSM 3353, JCM 31263), Eubacterium limosum (e.g., ATCC 8486, DSM 20543, JCM 6421), Eubacterium ramulus (e.g., ATCC 29099, DSM 15684, JCM), Methanobrevibacter smithii (e.g., ATCC 35061, DSM 861, JCM 328), Intestinibacter bartlettii (e.g., DSM 16795), Paraclostridium bifermentans (e.g., DSM 14991, JCM 1386) and any combination thereof; preferably selected from the species Intestinibacter bartlettii, Eubacterium limosum and Eubacterium ramulus. Preferably, the bacterial strain(s) being able to perform pathway (C2) may be selected from the species Acetobacterium carbinolicum (e.g., ATCC BAA-990, DSM 2925), Acetobacterium malicum (e.g., DSM 4132), Acetobacterium wieringae (e.g., ATCC 43740, DSM1911, JCM 2380), Blautia hydrogenotrophica (e.g., DSM 10507, JCM 14656), Blautia producta (e.g., ATCC 27340, DSM 2950, JCM 1471), Candidatus Methanomassiliicoccus intestinalis, Clostridium aceticum (e.g., ATCC 35044, DSM 1496, JCM 15732), Clostridium glycolicum ((e.g., ATCC 14880, DSM 1288, JCM 1401), Clostridium magnum (e.g., ATCC 49199, DSM 2767), Clostridium mayombe (e.g., ATCC 51428, DSM 2767), Anaerobutyricum/Eubacterium hallii (e.g., ATCC 27751, DSM 3353, JCM 31263), Eubacterium limosum (e.g., ATCC 8486, DSM 20543, JCM 6421), Eubacterium ramulus (e.g., ATCC 29099, DSM 15684, JCM) and Methanobrevibacter smithii (e.g., ATCC 35061, DSM 861, JCM 328) and any combination thereof; preferably selected from the species Anaerobutyricum/Eubacterium hallii, Blautia hydrogenotrophica, Blautia producta, Eubacterium limosum and Eubacterium ramulus.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
optionally one or several bacterial strain(s) selected from the genera Acidaminococcus, Agathobacter, Anaerobutyricum, Anaerostipes, Anaerotignum, Bariatricus, Clostridium, Clostridium_E, Clostridium_Q, Coprococcus, Coprococcus_A, Copromonas, Eubacterium, Faecalibacterium, Flavonifractor, Longicatena, Peptoniphilus and Roseburia; preferably from the genera Acidaminococcus, Anaerobutyricum, Anaerostipes, Coprococcus, Coprococcus_A, Copromonas, Eubacterium, Faecalibacterium, Flavonifractor, Longicatena and Roseburia; more preferably from the genera Acidaminococcus, Anaerobutyricum, Anaerostipes, Copromonas, Eubacterium, Faecalibacterium, Flavonifractor, Longicatena and Roseburia.
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
optionally one or several bacterial strain(s) selected from the genera Anaerotignum, Bacteroides, Phocaeicola and Veillonella; preferably the strains are from the genus Bacteroides or Phocaeicola; one or several bacterial strain(s) selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides and Prevotella;
one or several bacterial strain(s) selected from the genera Intestinibacter, Terrisporobacter, Paraclostridium, Blautia and Eubacterium; preferably from the genus Blautia or Intestinibacter;
Optionally one or several bacterial strain(s) selected from the genera Agathobacter, Anaerobutyricum, Anaerostipes, Anaerotignum, Desulfovibrio, Dysosmobacter, Faecalibacterium, Longicatena and Roseburia; preferably from the genera Agathobacter, Anaerobutyricum, Anaerostipes, Desulfovibrio, Faecalibacterium and Roseburia;
Optionally one or several bacterial strain(s) selected from the genera Anaerobutyricum, Anaerostipes and Eubacterium; preferably from the genera Anaerobutyricum and Anaerostipes;
Optionally one or several bacterial strain(s) selected from the genera Anaerotignum, Clostridium, Coprococcus_A, Frisingococcus and Veillonella; preferably from the genera Anaerotignum genus, Coprococcus_A, Frisingococcus and Veillonella; and/or
one or several bacterial strain(s) selected from the genera Dialister, Flavonifractor, Phascolarctobacterium, Phascolarctobacterium_A and Veillonella; preferably from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
optionally one or several bacterial strain(s) selected from the genera Acidaminococcus, Agathobacter, Anaerobutyricum, Anaerostipes, Anaerotignum, Bariatricus, Clostridium, Clostridium_E, Clostridium_Q, Coprococcus, Coprococcus_A, Copromonas, Eubacterium, Faecalibacterium, Flavonifractor, Longicatena, Peptoniphilus and Roseburia; preferably from the genera Acidaminococcus, Anaerobutyricum, Anaerostipes, Coprococcus, Coprococcus_A, Copromonas, Eubacterium, Faecalibacterium, Flavonifractor, Longicatena and Roseburia; more preferably from the genera Acidaminococcus, Anaerobutyricum, Anaerostipes, Copromonas, Eubacterium, Faecalibacterium, Flavonifractor, Longicatena and Roseburia.
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
optionally one or several bacterial strain(s) selected from the genera Anaerotignum, Bacteroides, Phocaeicola and Veillonella; preferably the strains are from the genus Bacteroides or Phocaeicola;
one or several bacterial strain(s) selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides and Prevotella;
one or several bacterial strain(s) selected from the genera Intestinibacter, Terrisporobacter, Paraclostridium, and Eubacterium; preferably from the genus Intestinibacter;
Optionally one or several bacterial strain(s) selected from the genera Agathobacter, Anaerobutyricum, Anaerostipes, Anaerotignum, Desulfovibrio, Dysosmobacter, Faecalibacterium, Longicatena and Roseburia; preferably from the genera Agathobacter, Anaerobutyricum, Anaerostipes, Desulfovibrio, Faecalibacterium and Roseburia;
Optionally one or several bacterial strain(s) selected from the genera Anaerobutyricum, Anaerostipes and Eubacterium; preferably from the genera Anaerobutyricum and Anaerostipes;
Optionally one or several bacterial strain(s) selected from the genera Anaerotignum, Clostridium, Coprococcus_A, Frisingococcus and Veillonella; preferably from the genera Anaerotignum genus, Coprococcus_A, Frisingococcus and Veillonella; and/or
one or several bacterial strain(s) selected from the genera Dialister, Flavonifractor, Phascolarctobacterium, Phascolarctobacterium_A and Veillonella; preferably from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
optionally one or several bacterial strain(s) selected from the genera Acidaminococcus, Agathobacter, Anaerobutyricum, Anaerostipes, Anaerotignum, Bariatricus, Clostridium, Clostridium_E, Clostridium_Q, Coprococcus, Coprococcus_A, Copromonas, Eubacterium, Faecalibacterium, Flavonifractor, Longicatena, Peptoniphilus and Roseburia; preferably from the genera Acidaminococcus, Anaerobutyricum, Anaerostipes, Coprococcus, Coprococcus_A, Copromonas, Eubacterium, Faecalibacterium, Flavonifractor, Longicatena and Roseburia; more preferably from the genera Acidaminococcus, Anaerobutyricum, Anaerostipes, Copromonas, Eubacterium, Faecalibacterium, Flavonifractor, Longicatena and Roseburia.
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
optionally one or several bacterial strain(s) selected from the genera Anaerotignum, Bacteroides, Phocaeicola and Veillonella; preferably the strains are from the genus Bacteroides or Phocaeicola;
one or several bacterial strain(s) selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides and Prevotella;
one or several bacterial strain(s) selected from the genera Blautia and Eubacterium; preferably from the genus Blautia;
Optionally one or several bacterial strain(s) selected from the genera Agathobacter, Anaerobutyricum, Anaerostipes, Anaerotignum, Desulfovibrio, Dysosmobacter, Faecalibacterium, Longicatena and Roseburia; preferably from the genera Agathobacter, Anaerobutyricum, Anaerostipes, Desulfovibrio, Faecalibacterium and Roseburia;
Optionally one or several bacterial strain(s) selected from the genera Anaerobutyricum, Anaerostipes and Eubacterium; preferably from the genera Anaerobutyricum and Anaerostipes;
Optionally one or several bacterial strain(s) selected from the genera Anaerotignum, Clostridium, Coprococcus_A, Frisingococcus and Veillonella; preferably from the genera Anaerotignum genus, Coprococcus_A, Frisingococcus and Veillonella; and/or
one or several bacterial strain(s) selected from the genera Dialister, Flavonifractor, Phascolarctobacterium, Phascolarctobacterium_A and Veillonella; preferably from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
one or several bacterial strain(s) selected from the genera Intestinibacter, Terrisporobacter, Paraclostridium bifermentans, Blautia and Eubacterium; preferably from the genus Blautia or Instinibacter.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella_A, Hungatella, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
one or several bacterial strain(s) selected from the genera Intestinibacter, Terrisporobacter, Blautia and Eubacterium; preferably from the genus Blautia or Terrisporobacter.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
one or several bacterial strain(s) selected from the genera Intestinibacter, Terrisporobacter, Paraclostridium bifermentans and Eubacterium; preferably from the genus Intestinibacter.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
one or several bacterial strain(s) selected from the genera Intestinibacter, Terrisporobacter, and Eubacterium; preferably from the genus Terrisporobacter.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus,
Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
one or several bacterial strain(s) selected from the genera Blautia and Eubacterium; preferably from the genus Blautia.

In a particular embodiment, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
one or several bacterial strain(s) selected from the genera Blautia and Terrisporobacter; preferably from the genus Blautia.

In some embodiments, such consortium further comprises iv):
one or several bacterial strain(s) being able to convert acetate into butyrate selected from the genera Agathobacter, Anaerobutyricum, Anaerostipes, Anaerotignum, Desulfovibrio, Dysosmobacter, Faecalibacterium, Longicatena and Roseburia; preferably from the genera Agathobacter, Anaerobutyricum, Anaerostipes, Desulfovibrio, Faecalibacterium and Roseburia;
one or several bacterial strain(s) being able to convert lactate into butyrate selected from the genera Anaerobutyricum, Anaerostipes and Eubacterium; preferably from the genera Anaerobutyricum and Anaerostipes; and/or
one or several bacterial strain(s) being able to convert primary substrates into butyrate selected from the genera Acidaminococcus, Agathobacter, Anaerobutyricum, Anaerostipes, Anaerotignum, Bariatricus, Clostridium, Clostridium_E, Clostridium_Q, Coprococcus, Coprococcus_A, Copromonas, Eubacterium, Faecalibacterium, Flavonifractor, Longicatena, Peptoniphilus and Roseburia; preferably from the genera Acidaminococcus, Anaerobutyricum, Anaerostipes, Coprococcus, Coprococcus_A, Copromonas, Eubacterium, Faecalibacterium, Flavonifractor, Longicatena and Roseburia; more preferably from the genera Acidaminococcus, Anaerobutyricum, Anaerostipes, Copromonas, Eubacterium, Faecalibacterium, Flavonifractor, Longicatena and Roseburia.

In particular, such consortium does not comprise a bacterial strain of the genus Clostridium.

Preferably, such consortium does not comprise:
one or more bacterial strain(s) selected from the species Anaerotignum nov sp., Clostridium_E sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella; even more preferably wherein the consortium does not comprise a bacterial strain able to convert lactate into propionate; and/or
one or more bacterial strain(s) selected from the species Anaerotignum lactatifermentans (e.g., DSM 14214), Anaerotignum sp000436415, Bacteroides fragilis (e.g., ATCC 25285, DSM 2151, JCM 11019), Phocaeicola vulgatus (e.g., ATCC 8482, DSM 1447, JCM 5826) and Veillonella ratti (e.g., ATCC 17746, DSM 20736, JCM 6512); preferably from Bacteroides fragilis (e.g., ATCC 25285, DSM 2151, JCM 11019) and Phocaeicola vulgatus (e.g., ATCC 8482, DSM 1447, JCM 5826); preferably selected from the genera Anaerotignum, Bacteroides, Phocaeicola and Veillonella; even more preferably wherein the consortium does not comprise a bacterial strain able to convert primary substrates into propionate.

In some embodiments, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
optionally one or several bacterial strain(s) selected from the genera Anaerotignum, Bacteroides, Phocaeicola and Veillonella; preferably the strains are from the genus Bacteroides or Phocaeicola;
one or several bacterial strain(s) selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides and Prevotella;
one or several bacterial strain(s) selected from the genera Intestinibacter, Terrisporobacter, Paraclostridium bifermentans, Blautia and Eubacterium; preferably from the genus Blautia or Intestinibacter; and
one or several bacterial strain(s) selected from the genera Dialister, Flavonifractor, Phascolarctobacterium and Veillonella; preferably from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

In some embodiments, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
optionally one or several bacterial strain(s) selected from the genera Anaerotignum, Bacteroides, Phocaeicola and Veillonella; preferably the strains are from the genus Bacteroides or Phocaeicola;
one or several bacterial strain(s) selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides and Prevotella;
one or several bacterial strain(s) selected from the genera Intestinibacter, Terrisporobacter, Blautia and Eubacterium; preferably from the genus Blautia or Terrisporobacter; and
one or several bacterial strain(s) selected from the genera Dialister, Flavonifractor, Phascolarctobacterium and Veillonella; preferably from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

In some embodiments, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus;
optionally one or several bacterial strain(s) selected from the genera Anaerotignum, Bacteroides, Phocaeicola and Veillonella; preferably the strains are from the genus Bacteroides or Phocaeicola;
one or several bacterial strain(s) selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides and Prevotella;
one or several bacterial strain(s) selected from the genera Intestinibacter, Terrisporobacter, Paraclostridium bifermentans, and Eubacterium; preferably from the genus Intestinibacter; and
one or several bacterial strain(s) selected from the genera Dialister, Flavonifractor, Phascolarctobacterium and Veillonella; preferably from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

In some embodiments, the consortium according to the invention comprises or consists of i) Agathobacter rectalis and ii) Anaerostipes caccae and/or Anaerobutyricum hallii and iii):
one or several bacterial strain(s) selected from the genera from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas; preferably from the genera Blautia, Coprococcus, Dorea, Erysipelatoclostridium, Faecalibacterium, Lachnospira and Ruminococcus; more preferably from the genera Dorea, Faecalibacterium and Ruminococcus, even more preferably from the genus Ruminococcus;
one or several bacterial strain(s) selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella; preferably from the genera Bifidobacterium, Blautia, Clostridium_Q, Clostridium_E, Desulfovibrio, Dorea, Eubacterium, Oliverpabstia and Rhiziobiaceae genus; more preferably from the genera Bifidobacterium, Blautia, Desulfovibrio, Dorea, Eubacterium and Rhiziobiaceae genus, even more preferably from the genus Bifidobacterium;
one or several bacterial strain(s) selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella; preferably from the genera Agathobacter, Bacteroides, Bariatricus, Collinsella, Enterococcus, Lacticaseibacillus/Lactobacillus, Peptostreptococcus, Streptococcus, and Sutterella; more preferably from the genera Agathobacter, Bacteroides, Bariatricus, Collinsella, Enterococcus, Lacticaseibacillus/Lactobacillus, Streptococcus and Sutterella, even more preferably from the genus Lactobacillus.
optionally one or several bacterial strain(s) selected from the genera Anaerotignum, Bacteroides, Phocaeicola and Veillonella; preferably the strains are from the genus Bacteroides or Phocaeicola;
one or several bacterial strain(s) selected from the genera Acutalibacter novel genus (in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides, Oliverbapstia, Parabacteroides, Phocaeicola and Prevotella; preferably from the genera Acutalibacter nov genus, Bacteroides, Parabacteroides, Phocaeicola and Prevotella; more preferably from the genera Acutalibacter nov genus, Bacteroides, Phocaeicola and Prevotella, even more preferably from the genera Bacteroides and Prevotella;
one or several bacterial strain(s) selected from the genera Blautia and Eubacterium; preferably from the genus Blautia; and
one or several bacterial strain(s) selected from the genera Dialister, Flavonifractor, Phascolarctobacterium and Veillonella; preferably from the genera Dialister, Flavonifractor, Phascolarctobacterium, Phascolarctobacterium_A and Veillonella, preferably from Dialister, Flavonifractor and Phascolarctobacterium and Phascolarctobacterium_A, more preferably from the genus Phascolarctobacterium.

In some embodiments, such consortium further comprises iv):
one or several bacterial strain(s) being able to convert acetate into butyrate selected from the genera Agathobacter, Anaerobutyricum, Anaerostipes, Anaerotignum, Desulfovibrio, Dysosmobacter, Faecalibacterium, Longicatena and Roseburia; preferably from the genera Agathobacter, Anaerobutyricum, Anaerostipes, Desulfovibrio, Faecalibacterium and Roseburia;
one or several bacterial strain(s) being able to convert lactate into butyrate selected from the genera Anaerobutyricum, Anaerostipes and Eubacterium; preferably from the genera Anaerobutyricum and Anaerostipes; and/or
one or several bacterial strain(s) being able to convert primary substrates into butyrate selected from the genera Acidaminococcus, Agathobacter, Anaerobutyricum, Anaerostipes, Anaerotignum, Bariatricus, Clostridium, Clostridium_E, Clostridium_Q, Coprococcus, Coprococcus_A, Copromonas, Eubacterium, Faecalibacterium, Flavonifractor, Longicatena, Peptoniphilus and Roseburia; preferably from the genera Acidaminococcus, Anaerobutyricum, Anaerostipes, Coprococcus, Coprococcus_A, Copromonas, Eubacterium, Faecalibacterium, Flavonifractor, Longicatena and Roseburia; more preferably from the genera Acidaminococcus, Anaerobutyricum, Anaerostipes, Copromonas, Eubacterium, Faecalibacterium, Flavonifractor, Longicatena and Roseburia.

Such consortium preferably does not comprise a bacterial strain of the genus Clostridium.

Preferably, such consortium does not comprise:
one or more bacterial strain(s) selected from the species Anaerotignum nov sp., Clostridium_E sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella; even more preferably wherein the consortium does not comprise a bacterial strain able to convert lactate into propionate; and/or
one or more bacterial strain(s) selected from the species Anaerotignum lactatifermentans (e.g., DSM 14214), Anaerotignum sp000436415, Bacteroides fragilis (e.g., ATCC 25285, DSM 2151, JCM 11019), Phocaeicola vulgatus (e.g., ATCC 8482, DSM 1447, JCM 5826) and Veillonella ratti (e.g., ATCC 17746, DSM 20736, JCM 6512); preferably from Bacteroides fragilis (e.g., ATCC 25285, DSM 2151, JCM 11019) and Phocaeicola vulgatus (e.g., ATCC 8482, DSM 1447, JCM 5826); preferably selected from the genera Anaerotignum, Bacteroides, Phocaeicola and Veillonella; even more preferably wherein the consortium does not comprise a bacterial strain able to convert primary substrates into propionate.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 3, 4 or 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
optionally one or several bacterial strain(s) selected from the species Acidaminococcus intestini (e.g., DSM 21505, CIP 108586), Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp000436415, Bariatricus comes (e.g., ATCC 27758), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Coprococcus eutactus (e.g., ATCC 27759), Coprococcus_A catus (e.g., ATCC 27761), Copromonas sp. (in particular having a 16SRNA sequence such as set forth in SEQ ID NO: 2, 3, 4 or any variant thereof having at least 97%, 98% or 99% sequence identity thereto), Eubacterium ramulus (e.g., ATCC 29099, DSM 15684, JCM 31355), Faecalibacterium prausnitzii (e.g., DSM 17677, ATCC 27768, ATCC 27766, JCM 31915), Flavonifractor plautii (e.g., ATCC 29863, DSM 4000), Longicatena sp. (in particular having a 16SRNA sequence such as set forth in SEQ ID NO: 11 and any variant thereof having at least 97%, 98% or 99% sequence identity thereto), Peptoniphilus vaginalis (e.g., DSM 101742), Roseburia hominis (e.g., DSM 16839) and Roseburia intestinalis (e.g., DSM 14610, JCM 31262); preferably from the species Acidaminococcus intestini, Anaerobutyricum/Eubacterium hallii, Anaerostipes caccae, Anaerostipes hadrus, Coprococcus eutactus, Coprococcus_A catus, Copromonas sp., Eubacterium ramulus, Faecalibacterium prausnitzii, Flavonifractor plautii, Longicatena sp., Roseburia hominis and Roseburia intestinalis; more preferably from the species Acidaminococcus intestini, Anaerobutyricum/Eubacterium hallii, Anaerostipes caccae, Anaerostipes hadrus, Eubacterium ramulus, Faecalibacterium prausnitzii, Flavonifractor plautii, Roseburia hominis and Roseburia intestinalis;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis;
one or several bacterial strain(s) selected from the species Acutalibacter nov genus (in particular having a 16SRNA sequence such as set forth in SEQ ID NO: 9 and any variant thereof having at least 97%, 98% or 99% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 and any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464); preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov genus, Bacteroides faecis, Bacteroides fragilis, Bacteroides thetaiotaomicron, Bacteroides xylanisolvens, Phocaeicola plebeius, Phocaeicola vulgatus and Prevotella copri;
one or several bacterial strain(s) selected from the species Intestinibacter bartlettii (e.g., DSM 16795), Terrisporobacter glycolicus (e.g., DSM 1288, ATCC 14880), Terrisporobacter mayombei (e.g., DSM 6539), Clostridium bifermentans (e.g., DSM 14991, JCM 1386), Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978), preferably Blautia hydrogenotrophica or Intestinibacter bartlettii;
optionally one or several bacterial strain(s) selected from the species Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp., Desulfovibrio piger (e.g., ATCC 29098, DSM 749), Dysosmobacter sp. (in particular having a 16SRNA sequence such as set forth in SEQ ID NO: 6 and any variant thereof having at least 97%, 98% or 99% sequence identity thereto), Faecalibacterium prausnitzii (e.g., ATCC 27768, ATCC 27766, DSM 17677, JCM 31915), Longicatena sp., Roseburia hominis (e.g., DSM 16839) and Roseburia intestinalis (e.g., DSM 14610, JCM 31262); preferably Agathobacter rectalis, Anaerobutyricum hallii, Anaerostipes caccae, Anaerostipes hadrus, Desulfovibirio piger, Faecalibacterium prausnitzii, Roseburia hominis and Roseburia intestinalis;
optionally one or several bacterial strain(s) selected from the species Anaerobutyricum/Eubacterium hallii (e.g., ATCC 27751, DSM 3353, JCM 31263), Anaerostipes caccae (e.g., DSM 14662, JCM 13470), Eubacterium callanderi (e.g., ATCC 49165, DSM 3662, JCM 10284) and Eubacterium limosum (e.g., ATCC 8486, DSM 20543, JCM 6421, JCM 9978); preferably from Anaerobutyricum hallii and Anaerostipes caccae;
optionally one or several bacterial strain(s) selected from the species Anaerotignum nov sp. (in particular having a 16SRNA sequence such as set forth in SEQ ID NO: 14 and any variant thereof having at least 97%, 98% or 99% sequence identity thereto), Clostridium_E sporosphaeroides (e.g., ATCC 25781, DSM 1294), Coprococcus_A catus (e.g., ATCC 27761), Frisingococcus sp. (in particular having a 16SRNA sequence such as set forth in SEQ ID NO: 15 and any variant thereof having at least 97%, 98% or 99% sequence identity thereto), Veillonella atypica (e.g., ATCC 17744, DSM 20739), Veillonella parvula subsp. Parvula (e.g., DSM 2008), Veillonella ratti (e.g., ATCC 17746, DSM 20736, JCM 6512), preferably from Anaerotignum nov sp., Coprococcus_A catus, Frisingococcus sp., Veillonella atypica and Veillonella parvula subsp. Parvula; and/or one or several bacterial strain(s) selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512), preferably from Dialister hominis, Dialister invisus, Flavonifractor plautii, Phascolarctobacterium faecium and Phascolarctobacterium_A succinatutens.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 4, 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
optionally one or several bacterial strain(s) selected from the species Acidaminococcus intestini (e.g., DSM 21505, CIP 108586), Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp000436415, Bariatricus comes (e.g., ATCC 27758), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Coprococcus eutactus (e.g., ATCC 27759), Coprococcus_A catus (e.g., ATCC 27761), Copromonas sp. (in particular having a 16SRNA sequence such as set forth in SEQ ID NO: 2, 4, 8, and any variant thereof having at least 97%, 98% or 99% sequence identity thereto), Eubacterium ramulus (e.g., ATCC 29099, DSM 15684, JCM 31355), Faecalibacterium prausnitzii (e.g., DSM 17677, ATCC 27768, ATCC 27766, JCM 31915), Flavonifractor plautii (e.g., ATCC 29863, DSM 4000), Longicatena sp. (in particular having a 16SRNA sequence such as set forth in SEQ ID NO: 11 and any variant thereof having at least 97%, 98% or 99% sequence identity thereto), Peptoniphilus vaginalis (e.g., DSM 101742), Roseburia hominis (e.g., DSM 16839) and Roseburia intestinalis (e.g., DSM 14610, JCM 31262); preferably from the species Acidaminococcus intestini, Anaerobutyricum/Eubacterium hallii, Anaerostipes caccae, Anaerostipes hadrus, Coprococcus eutactus, Coprococcus_A catus, Copromonas sp., Eubacterium ramulus, Faecalibacterium prausnitzii, Flavonifractor plautii, Longicatena sp., Roseburia hominis and Roseburia intestinalis; more preferably from the species Acidaminococcus intestini, Anaerobutyricum/Eubacterium hallii, Anaerostipes caccae, Anaerostipes hadrus, Eubacterium ramulus, Faecalibacterium prausnitzii, Flavonifractor plautii, Roseburia hominis and Roseburia intestinalis;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, f Enterococcus faecium, Enterococcus gallinarum, Lacticaseibacillus rhamnosus/Lactobacillus rhamnosus, Streptococcus anginosus and Sutterella wadsworthensis;
one or several bacterial strain(s) selected from the species Acutalibacter nov genus (in particular having a 16SRNA sequence such as set forth in SEQ ID NO: 9 and any variant thereof having at least 97%, 98% or 99% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 and any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464); preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov genus, Bacteroides faecis, Bacteroides fragilis, Bacteroides thetaiotaomicron, Bacteroides xylanisolvens, Phocaeicola plebeius, Phocaeicola vulgatus and Prevotella copri;
one or several bacterial strain(s) selected from the species Intestinibacter bartlettii (e.g., DSM 16795), Terrisporobacter glycolicus (e.g., DSM 1288, ATCC 14880), Terrisporobacter mayombei (e.g., DSM 6539), Clostridium bifermentans (e.g., DSM 14991, JCM 1386), Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978), preferably Blautia hydrogenotrophica or Intestinibacter bartlettii;
optionally one or several bacterial strain(s) selected from the species Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp., Desulfovibrio piger (e.g., ATCC 29098, DSM 749), Dysosmobacter sp. (in particular having a 16SRNA sequence such as set forth in SEQ ID NO: 6 and any variant thereof having at least 97%, 98% or 99% sequence identity thereto), Faecalibacterium prausnitzii (e.g., ATCC 27768, ATCC 27766, DSM 17677, JCM 31915), Longicatena sp., Roseburia hominis (e.g., DSM 16839) and Roseburia intestinalis (e.g., DSM 14610, JCM 31262); preferably Agathobacter rectalis, Anaerobutyricum hallii, Anaerostipes caccae, Anaerostipes hadrus, Desulfovibirio piger, Faecalibacterium prausnitzii, Roseburia hominis and Roseburia intestinalis;
optionally one or several bacterial strain(s) selected from the species Anaerobutyricum/Eubacterium hallii (e.g., ATCC 27751, DSM 3353, JCM 31263), Anaerostipes caccae (e.g., DSM 14662, JCM 13470), Eubacterium callanderi (e.g., ATCC 49165, DSM 3662, JCM 10284) and Eubacterium limosum (e.g., ATCC 8486, DSM 20543, JCM 6421, JCM 9978); preferably from Anaerobutyricum hallii and Anaerostipes caccae;
optionally one or several bacterial strain(s) selected from the species Anaerotignum nov sp. (in particular having a 16SRNA sequence such as set forth in SEQ ID NO: 14 and any variant thereof having at least 97%, 98% or 99% sequence identity thereto), Clostridium_E sporosphaeroides (e.g., ATCC 25781, DSM 1294), Coprococcus_A catus (e.g., ATCC 27761), Frisingococcus sp. (in particular having a 16SRNA sequence such as set forth in SEQ ID NO: 15 and any variant thereof having at least 97%, 98% or 99% sequence identity thereto), Veillonella atypica (e.g., ATCC 17744, DSM 20739), Veillonella parvula subsp. Parvula (e.g., DSM 2008), Veillonella ratti (e.g., ATCC 17746, DSM 20736, JCM 6512), preferably from Anaerotignum nov sp., Coprococcus_A catus, Frisingococcus sp., Veillonella atypica and Veillonella parvula subsp. Parvula; and/or
one or several bacterial strain(s) selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512), preferably from Dialister hominis, Dialister invisus, Flavonifractor plautii, Phascolarctobacterium faecium and Phascolarctobacterium_A succinatutens.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 4, 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
optionally one or several bacterial strain(s) selected from the species Acidaminococcus intestini (e.g., DSM 21505, CIP 108586), Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp000436415, Bariatricus comes (e.g., ATCC 27758), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Coprococcus eutactus (e.g., ATCC 27759), Coprococcus_A catus (e.g., ATCC 27761), Copromonas sp. (in particular having a 16SRNA sequence such as set forth in SEQ ID NO: 2, 4, 8, and any variant thereof having at least 97%, 98% or 99% sequence identity thereto), Eubacterium ramulus (e.g., ATCC 29099, DSM 15684, JCM 31355), Faecalibacterium prausnitzii (e.g., DSM 17677, ATCC 27768, ATCC 27766, JCM 31915), Flavonifractor plautii (e.g., ATCC 29863, DSM 4000), Longicatena sp. (in particular having a 16SRNA sequence such as set forth in SEQ ID NO: 11 and any variant thereof having at least 97%, 98% or 99% sequence identity thereto), Peptoniphilus vaginalis (e.g., DSM 101742), Roseburia hominis (e.g., DSM 16839) and Roseburia intestinalis (e.g., DSM 14610, JCM 31262); preferably from the species Acidaminococcus intestini, Anaerobutyricum/Eubacterium hallii, Anaerostipes caccae, Anaerostipes hadrus, Coprococcus eutactus, Coprococcus_A catus, Copromonas sp., Eubacterium ramulus, Faecalibacterium prausnitzii, Flavonifractor plautii, Longicatena sp., Roseburia hominis and Roseburia intestinalis; more preferably from the species Acidaminococcus intestini, Anaerobutyricum/Eubacterium hallii, Anaerostipes caccae, Anaerostipes hadrus, Eubacterium ramulus, Faecalibacterium prausnitzii, Flavonifractor plautii, Roseburia hominis and Roseburia intestinalis;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis;
one or several bacterial strain(s) selected from the species Acutalibacter nov genus (in particular having a 16SRNA sequence such as set forth in SEQ ID NO: 9 and any variant thereof having at least 97%, 98% or 99% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 and any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464); preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov genus, Bacteroides faecis, Bacteroides fragilis, Bacteroides thetaiotaomicron, Bacteroides xylanisolvens, Phocaeicola plebeius, Phocaeicola vulgatus and Prevotella copri;
one or several bacterial strain(s) selected from the species Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978), preferably Blautia hydrogenotrophica;
optionally one or several bacterial strain(s) selected from the species Agathobacter rectalis/Eubacterium rectale (e.g., DSM 17629), Agathobacter/Roseburia faecis (e.g., DSM 16840), Anaerobutyricum/Eubacterium hallii (e.g., ATCC 27751, DSM 3353, JCM 31263), Anaerostipes caccae (e.g., DSM 14662, JCM 13470), Anaerostipes hadrus (e.g., ATCC 29173, DSM 3319), Anaerotignum sp., Desulfovibrio piger (e.g., ATCC 29098, DSM 749), Dysosmobacter sp. (in particular having a 16SRNA sequence such as set forth in SEQ ID NO: 6 and any variant thereof having at least 97%, 98% or 99% sequence identity thereto), Faecalibacterium prausnitzii (e.g., ATCC 27768, ATCC 27766, DSM 17677, JCM 31915), Longicatena sp., Roseburia hominis (e.g., DSM 16839) and Roseburia intestinalis (e.g., DSM 14610, JCM 31262); preferably Agathobacter rectalis, Anaerobutyricum hallii, Anaerostipes caccae, Anaerostipes hadrus, Desulfovibirio piger, Faecalibacterium prausnitzii, Roseburia hominis and Roseburia intestinalis;
optionally one or several bacterial strain(s) selected from the species Anaerobutyricum/Eubacterium hallii (e.g., ATCC 27751, DSM 3353, JCM 31263), Anaerostipes caccae (e.g., DSM 14662, JCM 13470), Eubacterium callanderi (e.g., ATCC 49165, DSM 3662, JCM 10284) and Eubacterium limosum (e.g., ATCC 8486, DSM 20543, JCM 6421, JCM 9978); preferably from Anaerobutyricum hallii and Anaerostipes caccae;
optionally one or several bacterial strain(s) selected from the species Anaerotignum nov sp. (in particular having a 16SRNA sequence such as set forth in SEQ ID NO: 14 and any variant thereof having at least 97%, 98% or 99% sequence identity thereto), Clostridium_E sporosphaeroides (e.g., ATCC 25781, DSM 1294), Coprococcus_A catus (e.g., ATCC 27761), Frisingococcus sp. (in particular having a 16SRNA sequence such as set forth in SEQ ID NO: 15 and any variant thereof having at least 97%, 98% or 99% sequence identity thereto), Veillonella atypica (e.g., ATCC 17744, DSM 20739), Veillonella parvula subsp. Parvula (e.g., DSM 2008), Veillonella ratti (e.g., ATCC 17746, DSM 20736, JCM 6512), preferably from Anaerotignum nov sp., Coprococcus_A catus, Frisingococcus sp., Veillonella atypica and Veillonella parvula subsp. Parvula; and/or
one or several bacterial strain(s) selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512), preferably from Dialister hominis, Dialister invisus, Flavonifractor plautii, Phascolarctobacterium faecium and Phascolarctobacterium_A succinatutens.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 3, 4 or 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis; and
one or several bacterial strain(s) selected from the species Intestinibacter bartlettii (e.g., DSM 16795), Terrisporobacter glycolicus (e.g., DSM 1288, ATCC 14880), Terrisporobacter mayombei (e.g., DSM 6539), Clostridium bifermentans (e.g., DSM 14991, JCM 1386), Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978), preferably Intestinibacter bartlettii or Blautia hydrogenotrophica.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 4, 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, longum, Bifidobacterium Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis; and
one or several bacterial strain(s) selected from the species Intestinibacter bartlettii (e.g., DSM 16795), Terrisporobacter glycolicus (e.g., DSM 1288, ATCC 14880), Terrisporobacter mayombei (e.g., DSM 6539), Clostridium bifermentans (e.g., DSM 14991, JCM 1386), Eubacterium callanderi (e.g., ATCC 49165, DSM 3662, JCM 10284) and Eubacterium limosum (e.g., ATCC 8486, DSM 20543, JCM 6421, JCM 9978), preferably Intestinibacter bartlettii.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 4, 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; preferably from the species Bifidobacterium adolescentis, Bifidobacterium catenulatum, more Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis; and
one or several bacterial strain(s) selected from the species Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978), preferably Blautia hydrogenotrophica.

In some embodiments, such consortium further comprises iv):
one or several bacterial strain(s) be selected from the species Agathobacter/Rosebura feacis (e.g., DSM 16840), Anaerostipes hadrus (e.g., ATCC 29173, DSM 3319), Anaerotignum lactatifermentans (e.g., DSM 14214), Eubacterium ramulus (e.g., ATCC 29099, DSM 15684, JCM 31355), Faecalibacterium prausnitzii (e.g., ATCC 27768, ATCC 27766, DSM 17677, JCM 31915), Roseburia hominis (e.g., DSM 16839) and Roseburia intestinalis (e.g., DSM 14610, CIP 107878, JCM 31262);
one or several bacterial strain(s) selected from the Eubacterium callanderi, DSM 755, JCM 1380), Eubacterium limosum (e.g., ATCC 8486, DSM 20543, JCM 6421) and Eubacterium ramulus (e.g., ATCC 29099, DSM 15684, JCM 31355);
one or several bacterial strain(s) selected from the species Acidaminococcus intestine, Faecalibacterium prausnitzii, Flavonifractor plautii (e.g., ATCC 29863, DSM 4000), Flintibacter butyricum (e.g., DSM 27579), Longicatena nov. sp., Roseburia intestinalis.

In particular, such consortium does not comprise a bacterial strain of the genus Clostridium.

Preferably, such consortium does not comprise:
one or more bacterial strain(s) selected from the species Anaerotignum nov sp., Clostridium_E sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella; even more preferably wherein the consortium does not comprise a bacterial strain able to convert lactate into propionate; and/or
one or more bacterial strain(s) selected from the species Anaerotignum lactatifermentans (e.g., DSM 14214), Anaerotignum sp000436415, Bacteroides fragilis (e.g., ATCC 25285, DSM 2151, JCM 11019), Phocaeicola vulgatus (e.g., ATCC 8482, DSM 1447, JCM 5826) and Veillonella ratti (e.g., ATCC 17746, DSM 20736, JCM 6512); preferably from Bacteroides fragilis (e.g., ATCC 25285, DSM 2151, JCM 11019) and Phocaeicola vulgatus (e.g., ATCC 8482, DSM 1447, JCM 5826); preferably selected from the genera Anaerotignum, Bacteroides, Phocaeicola and Veillonella; even more preferably wherein the consortium does not comprise a bacterial strain able to convert primary substrates into propionate.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 3, 4 or 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, longum, Bifidobacterium Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis;
one or several bacterial strain(s) selected from the species Acutalibacter nov genus (in particular having a 16SRNA sequence such as set forth in SEQ ID NO: 9 and any variant thereof having at least 97%, 98% or 99% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 and any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464); preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov genus, Bacteroides faecis, Bacteroides fragilis, Bacteroides thetaiotaomicron, Bacteroides xylanisolvens, Phocaeicola plebeius, Phocaeicola vulgatus and Prevotella copri;
one or several bacterial strain(s) selected from the species Intestinibacter bartlettii (e.g., DSM 16795), Terrisporobacter glycolicus (e.g., DSM 1288, ATCC 14880), Terrisporobacter mayombei (e.g., DSM 6539), Clostridium bifermentans (e.g., DSM 14991, JCM 1386), Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978), preferably Intestinibacter bartlettii or Blautia hydrogenotrophica; and
one or several bacterial strain(s) selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512), preferably from Dialister hominis, Dialister invisus, Flavonifractor plautii, Phascolarctobacterium faecium and Phascolarctobacterium_A succinatutens.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 4, 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis;
one or several bacterial strain(s) selected from the species Acutalibacter nov genus (in particular having a 16SRNA sequence such as set forth in SEQ ID NO: 9 and any variant thereof having at least 97%, 98% or 99% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 and any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464); preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov genus, Bacteroides faecis, Bacteroides fragilis, Bacteroides thetaiotaomicron, Bacteroides xylanisolvens, Phocaeicola plebeius, Phocaeicola vulgatus and Prevotella copri;
one or several bacterial strain(s) selected from the species Intestinibacter bartlettii (e.g., DSM 16795), Terrisporobacter glycolicus (e.g., DSM 1288, ATCC 14880), Terrisporobacter mayombei (e.g., DSM 6539), Clostridium bifermentans (e.g., DSM 14991, JCM 1386Eubacterium callanderi (e.g., ATCC 49165, DSM 3662, JCM 10284) and Eubacterium limosum (e.g., ATCC 8486, DSM 20543, JCM 6421, JCM 9978), preferably Intestinibacter bartlettii; and
one or several bacterial strain(s) selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512), preferably from Dialister hominis, Dialister invisus, Flavonifractor plautii, Phascolarctobacterium faecium and Phascolarctobacterium_A succinatutens.

In a particular embodiment, the consortium according to the invention comprises or consists of i) Agathobacter rectalis and ii) Anaerostipes caccae and/or Anaerobutyricum hallii and iii):
one or several bacterial strain(s) selected from the species Anaerobutyricum/Eubacterium hallii (e.g., ATCC 27751, DSM 3353, JCM 31263), Bacteroides clarus (e.g., DSM 22519, JCM 16067), Blautia hydrogenotrophica (e.g., DSM 10507, JCM 14656), Collinsella aerofaciens (e.g., ATCC 25986, DSM 3979, JCM 10188), Coprococcus eutactus (e.g., ATCC 27759), Dorea formicigenerans (e.g., ATCC 27755, DSM 3992, JCM 31256), Dorea longicatena (e.g., DSM 13814, JCM 11232), Erysipelatoclostridium ramosum (e.g., ATCC 25582, DSM 1402, JCM 1298), Eubacterium ramulus (e.g., ATCC 29099, DSM 15684, JCM 31355), Faecalibacterium prausnitzii (e.g., DSM 17677, ATCC 27768, ATCC 27766, JCM 31915), Lachnospira (Eubacterium) eligens (e.g., ATCC 27750, DSM 3376), Longicatena sp. (in particular having a 16SRNA sequence such as set forth in SEQ ID NO: 11 and any variant thereof having at least 97%, 98% or 99% sequence identity thereto), Ruminococcus bromii (e.g., ATCC 27255) and Sellimonas intestinalis (e.g., JCM 30749); preferably from the species, Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii; more preferably from the species Dorea formicigenerans, Dorea longicatena, Faecalibacterium prausnitzii and Ruminococcus bromii;
one or several bacterial strain(s) selected from the species Acidaminococcus intestini (e.g., DSM 21505), Acutalibacter nov. genus (in particular having a 16SRNA sequence such as set forth in SEQ ID NO: 9 and any variant thereof having at least 97%, 98% or 99% sequence identity thereto), Bifidobacterium adolescentis (e.g., ATCC 15703, DSM 20083, JCM 1251), Bifidobacterium catenulatum (e.g., ATCC 27539, DSM 16992, JCM 1194), Bifidobacterium longum (e.g., ATCC 15707, DSM 20219, JCM 1217), Bifidobacterium pseudocatenulatum (e.g., ATCC 27919, DSM 20438, JCM 1200), Bifidobacterium ruminantium (e.g., ATCC 49390, DSM 6489), Blautia hydrogenotrophica (e.g., DSM 10507, JCM 14656), Clostridium perfringens (e.g., ATCC 13124, DSM 756), Clostridium_E sporosphaeroides (e.g., ATCC 25781, DSM 1294), Clostridium_Q symbiosum (e.g., ATCC 14940, DSM 934, JCM 1297), Collinsella aerofaciens (e.g., ATCC 25986, DSM 3979, JCM 10188), Copromonas sp. (in particular having a 16SRNA sequence such as set forth in SEQ ID NO: 2, 4, 8, and any variant thereof having at least 97%, 98% or 99% sequence identity thereto), Desulfovibrio piger (e.g., ATCC 29098, DSM 749), Dorea formicigenerans (e.g., ATCC 27755, DSM 3992, JCM 31256), Dorea longicatena (e.g., DSM 13814, JCM 11232), Dorea scindens, Dorea sp. 900066555, Enterocloster sp. (in particular having a 16SRNA sequence such as set forth in SEQ ID NO: 12 and any variant thereof having at least 97%, 98% or 99% sequence identity thereto), Escherichia coli (e.g., ATCC 11775, DSM 30083, JCM 1649), Eubacterium callanderi (e.g., ATCC 49165, DSM 3662, JCM 10284), Eubacterium limosum (e.g., ATCC 8486, DSM 20543, JCM 6421, JCM 9978), Hungatella effluvii (e.g., DSM 24995), Hungatella_A sp. (in particular having a 16SRNA sequence such as set forth in SEQ ID NO: 5 and any variant thereof having at least 97%, 98% or 99% sequence identity thereto), Oliverpabstia sp. (in particular having a 16SRNA sequence such as set forth in SEQ ID NO: 7 and any variant thereof having at least 97%, 98% or 99% sequence identity thereto), Peptoniphilus vaginalis (e.g., DSM 101742), Peptostreptococcus anaerobius (e.g., ATCC 27337, DSM 2949), Phocaeicola plebeius (e.g., DSM 17135, JCM 12973), Rhizobiaceae nov. genus, (in particular having a 16SRNA sequence such as set forth in SEQ ID NO: 13 and any variant thereof having at least 97%, 98% or 99% sequence identity thereto), Ruminoccocus bromii (e.g., ATCC 27255), Sellimonas intestinalis (e.g., JCM 30749) and Veillonella ratti (e.g., ATCC 17746, DSM 20736, JCM 6512); preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp. 900066555, Eubacterium callanderi, Eubacterium limosum, Oliverpabstia sp. And Rhizobiaceae nov. genus; more preferably from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum, and Rhizobiaceae nov. genus;
one or several bacterial strain(s) selected from the species Agathobacter rectalis/Eubacterium rectale (e.g., DSM 17629), Agathobacter/Roseburia faecis (e.g., DSM 16840), Bacteroides clarus (e.g., DSM 22519, JCM 16067), Bacteroides faecis (e.g., DSM 24798, JCM 16478), Bacteroides uniformis (e.g., DSM 6597, ATCC 8492, JCM5828), Bariatricus comes (e.g., ATCC 27758), Bifidobacterium adolescentis (e.g., ATCC 15703, DSM 20083, JCM 1251), Collinsella aerofaciens (e.g., ATCC 25986, DSM 3979, JCM 10188), Enterococcus faecalis (e.g., ATCC 29212, DSM 2570), Enterococcus faecium (e.g., ATCC 19434, DSM 10477, JCM 8727), Enterococcus gallinarum (e.g., ATCC 49573, JCM 8728), Lacticaseibacillus rhamnosus/Lactobacillus rhamnosus (e.g., ATCC 7469, DSM 20021, JCM1136), Longicatena sp. (in particular having a 16SRNA sequence such as set forth in SEQ ID NO: 11 and any variant thereof having at least 97%, 98% or 99% sequence identity thereto), Merdisoma Aerosacheriphilus (in particular having a 16SRNA sequence such as set forth in SEQ ID NO: 10 and any variant thereof having at least 97%, 98% or 99% sequence identity thereto), Peptostreptococcus stomatis (e.g., DSM 17678, JCM 15636), Roseburia hominis (e.g., DSM 16839), Streptococcus anginosus (e.g., ATCC 33397, DSM 20563, JCM 12993), Sutterella wadsworthensis (e.g., ATCC 51579, DSM 14016); preferably from the species Agathobacter rectalis/Eubacterium rectale, Agathobacter/Roseburia faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Peptostreptococcus stomatis, Streptococcus anginosus and Sutterella wadsworthensis; more preferably from the species Agathobacter rectalis/Eubacterium rectale, Bacteroides uniformis, Bariatricus comes, Collinsella aerofaciens, Enterococcus faecalis, Enterococcus faecium, Enterococcus gallinarum, Lacticaseibacillus rhamnosus/Lactobacillus rhamnosus, Streptococcus anginosus and Sutterella wadsworthensis;
one or several bacterial strain(s) selected from the species Acutalibacter nov genus (in particular having a 16SRNA sequence such as set forth in SEQ ID NO: 9 and any variant thereof having at least 97%, 98% or 99% sequence identity thereto), Bacteroides faecis (e.g., DSM 24798, JCM 16478), Bacteroides fragilis (e.g., ATCC 25285, DSM 2151, JCM 11019), Bacteroides thetaiotaomicron (e.g., ATCC 29148, DSM 2079, JCM 5827), Bacteroides xylanisolvens (e.g., DSM 18836, JCM 15633), Oliverpabstia sp. (in particular having a 16SRNA sequence such as set forth in SEQ ID NO: 7 and any variant thereof having at least 97%, 98% or 99% sequence identity thereto), Bacteroides faecis (e.g., DSM 24798, JCM 16478), Parabacteroides distasonis (e.g., ATCC 8503, DSM 20701), Phocaeicola dorei (e.g., DSM 17855, JCM 13471), Phocaeicola plebeius (e.g., DSM 17135, JCM 12973), Phocaeicola vulgatus (e.g., ATCC 8482, DSM 1447, JCM 5826) and Prevotella copri (e.g., DSM 18205, JCM 13464); preferably from Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri; more preferably from the species Acutalibacter nov genus, Bacteroides faecis, Bacteroides fragilis, Bacteroides thetaiotaomicron, Bacteroides xylanisolvens, Phocaeicola plebeius, Phocaeicola vulgatus and Prevotella copri;
one or several bacterial strain(s) selected from the species Blautia hydrogenotrophica (e.g., DSM 10507, JCM 14656), Eubacterium callanderi (e.g., ATCC 49165, DSM 3662, JCM 10284) and Eubacterium limosum (e.g., ATCC 8486, DSM 20543, JCM 6421, JCM 9978), preferably Blautia hydrogenotrophica; and one or several bacterial strain(s) selected from the species Dialister hominis (e.g., DSM 109768), Dialister invisus (e.g., DSM 15470, JCM 17566), Flavonifractor plautii (e.g., ATCC 29863, DSM 4000), Phascolarctobacterium faecium (e.g., DSM 14760), Phascolarctobacterium_A succinatutens (e.g., DSM 22533), Veillonella parvula subsp. Parvula (e.g., DSM 2008) and Veillonella ratti (e.g., ATCC 17746, DSM 20736, JCM 6512), preferably from Dialister hominis, Dialister invisus, Flavonifractor plautii, Phascolarctobacterium faecium and Phascolarctobacterium_A succinatutens.

In some embodiments, such consortium further comprises iv):
one or several bacterial strain(s) be selected from the species Agathobacter/Rosebura feacis (e.g., DSM 16840), Anaerostipes hadrus (e.g., ATCC 29173, DSM 3319), Anaerotignum lactatifermentans (e.g., DSM 14214), Eubacterium ramulus (e.g., ATCC 29099, DSM 15684, JCM 31355), Faecalibacterium prausnitzii (e.g., ATCC 27768, ATCC 27766, DSM 17677, JCM 31915), Roseburia hominis (e.g., DSM 16839) and Roseburia intestinalis (e.g., DSM 14610, CIP 107878, JCM 31262);
one or several bacterial strain(s) selected from the Eubacterium callanderi, (e.g., DSM 755, JCM 1380), Eubacterium limosum (e.g., ATCC 8486, DSM 20543, JCM 6421) and Eubacterium ramulus (e.g., ATCC 29099, DSM 15684, JCM 31355);
one or several bacterial strain(s) selected from the species Acidaminococcus intestine, Faecalibacterium prausnitzii, Flavonifractor plautii (e.g., ATCC 29863, DSM 4000), Flintibacter butyricum (e.g., DSM 27579), Longicatena nov. sp., Roseburia intestinalis.

In particular, such consortium does not comprise a bacterial strain of the genus Clostridium.

Preferably, such consortium does not comprise:
one or more bacterial strain(s) selected from the species Anaerotignum nov sp., Clostridium_E sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella; even more preferably wherein the consortium does not comprise a bacterial strain able to convert lactate into propionate; and/or one or more bacterial strain(s) selected from the species Anaerotignum lactatifermentans (e.g., DSM 14214), Anaerotignum sp000436415, Bacteroides fragilis (e.g., ATCC 25285, DSM 2151, JCM 11019), Phocaeicola vulgatus (e.g., ATCC 8482, DSM 1447, JCM 5826) and Veillonella ratti (e.g., ATCC 17746, DSM 20736, JCM 6512); preferably from Bacteroides fragilis (e.g., ATCC 25285, DSM 2151, JCM 11019) and Phocaeicola vulgatus (e.g., ATCC 8482, DSM 1447, JCM 5826); preferably selected from the genera Anaerotignum, Bacteroides, Phocaeicola and Veillonella; even more preferably wherein the consortium does not comprise a bacterial strain able to convert primary substrates into propionate.

In a particular embodiment, the consortium according to the invention comprises or consists of i) Agathobacter rectalis and ii) Anaerostipes caccae and/or Anaerobutyricum hallii and iii) a bacterium of the genera Ruminococcus; a bacterium of the genera Bifidobacterium; a bacterium of the genera Lactobacillus; a bacterium of the genera Blautia; a bacterium of the genera Phascolarctobacterium and a bacterium of the genera Bacteroides or Prevotella.

In particular, such consortium does not comprise a bacterial strain of the genus Clostridium.

Preferably, such consortium does not comprise:
one or more bacterial strain(s) selected from the species Anaerotignum nov sp., Clostridium_E sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella; even more preferably wherein the consortium does not comprise a bacterial strain able to convert lactate into propionate; and/or 1 one or more bacterial strain(s) selected from the species Anaerotignum lactatifermentans (e.g., DSM 14214), Anaerotignum sp000436415, Bacteroides fragilis (e.g., ATCC 25285, DSM 2151, JCM 11019), Phocaeicola vulgatus (e.g., ATCC 8482, DSM 1447, JCM 5826) and Veillonella ratti (e.g., ATCC 17746, DSM 20736, JCM 6512); preferably from Bacteroides fragilis (e.g., ATCC 25285, DSM 2151, JCM 11019) and Phocaeicola vulgatus (e.g., ATCC 8482, DSM 1447, JCM 5826); preferably selected from the genera Anaerotignum, Bacteroides, Phocaeicola and Veillonella; even more preferably wherein the consortium does not comprise a bacterial strain able to convert primary substrates into propionate.

In a particular embodiment, the consortium according to the invention comprises or consists of i) Agathobacter rectalis and ii) Anaerostipes caccae and/or Anaerobutyricum hallii, iii) a bacterium of the genus Ruminococcus; a bacterium of the genus Bifidobacterium; a bacterium of the genus Lactobacillus; a bacterium of the genus Phascolarctobacterium and a bacterium of the genus Bacteroides or Prevotella, and iv) a bacterium of the genus Blautia, Eubacterium, Paraclostridium, Terrisporobacter or Intestinibacter.

In a particular embodiment, the consortium according to the invention comprises or consists of i) Agathobacter rectalis and ii) Anaerostipes caccae and/or Anaerobutyricum hallii, iii) a bacterium of the genus Ruminococcus; a bacterium of the genus Bifidobacterium; a bacterium of the genus Lactobacillus; a bacterium of the genus Phascolarctobacterium and a bacterium of the genus Bacteroides or Prevotella, and iv) a bacterium of the genus Eubacterium, Paraclostridium, Terrisporobacter or Intestinibacter.

In a particular embodiment, the consortium according to the invention comprises or consists of i) Agathobacter rectalis and ii) Anaerostipes caccae and/or Anaerobutyricum hallii, iii) a bacterium of the genus Ruminococcus; a bacterium of the genus Bifidobacterium; a bacterium of the genus Lactobacillus; a bacterium of the genus Phascolarctobacterium and a bacterium of the genus Bacteroides or Prevotella, and iv) a bacterium of the genus Eubacterium.

In a particular embodiment, the consortium according to the invention comprises or consists of i) Agathobacter rectalis and ii) Anaerostipes caccae and/or Anaerobutyricum hallii, iii) a bacterium of the genus Ruminococcus; a bacterium of the genus Bifidobacterium; a bacterium of the genus Lactobacillus; a bacterium of the genus Phascolarctobacterium and a bacterium of the genus Bacteroides or Prevotella, and iv) a bacterium of the genus Paraclostridium.

In a particular embodiment, the consortium according to the invention comprises or consists of i) Agathobacter rectalis and ii) Anaerostipes caccae and/or Anaerobutyricum hallii, iii) a bacterium of the genus Ruminococcus; a bacterium of the genus Bifidobacterium; a bacterium of the genus Lactobacillus; a bacterium of the genus Phascolarctobacterium and a bacterium of the genus Bacteroides or Prevotella, and iv) a bacterium of the genus Terrisporobacter.

In a particular embodiment, the consortium according to the invention comprises or consists of i) Agathobacter rectalis and ii) Anaerostipes caccae and/or Anaerobutyricum hallii, iii) a bacterium of the genus Ruminococcus; a bacterium of the genus Bifidobacterium; a bacterium of the genus Lactobacillus; a bacterium of the genus Phascolarctobacterium and a bacterium of the genus Bacteroides or Prevotella, and iv) a bacterium of the genus Intestinibacter.

In some embodiments, the consortium according to the invention comprises or consists of i) Agathobacter rectalis, ii) Anaerostipes caccae and/or Anaerobutyricum hallii, iii) Lactobacillus rhamnosus and iv)
one or several bacterial strain(s) able to convert formate into acetate such as described here above; and/or
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Blautia hydrogenotrophica and iv)
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Blautia hydrogenotrophica and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Blautia hydrogenotrophica and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Ruminococcus bromii and iv)
one or several bacterial strain(s) able to convert primary substrates into lactate such as described here above; and/or
one or several bacterial strain(s) able to convert formate into acetate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Blautia hydrogenotrophica and iv)
one or several bacterial strain(s) able to convert primary substrates into lactate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert primary substrates into formate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Ruminococcus bromii and Blautia hydrogenotrophica and iv)
one or several bacterial strain(s) able to convert primary substrates into lactate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Ruminococcus bromii, Blautia hydrogenotrophica and Bifidobacterium adolescentis and iv)
one or several bacterial strain(s) able to convert primary substrates into lactate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Bifidobacterium adolescentis and iv)
one or several bacterial strain(s) able to convert primary substrates into formate such as described here above; and/or
one or several bacterial strain(s) able to convert primary substrates into lactate such as described here above; and/or
one or several bacterial strain(s) able to convert formate into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Paraclostridium bifermentans and iv)
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter othiniensis and iv)
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Blautia_A wexlerae (in particular having a 16SRNA sequence as set forth in SEQ ID NO: 17 and any variant thereof having at least 97%, 98% or 99% sequence identity thereto), and iv)
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter glycolicus and iv)
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter mayombei and iv)
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Intestinibacter bartlettii and iv)
one or several bacterial strain(s) able to convert primary substrates into formate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above. In some embodiments, the consortium according to the invention comprises or consists of i) Agathobacter rectalis, ii) Anaerostipes caccae and/or Anaerobutyricum hallii, iii) Lactobacillus rhamnosus and Blautia hydrogenotrophica and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Paraclostridium bifermentans and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter othiniensis and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Blautia_A wexlerae and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter glycolicus and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter mayombei and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Intestinibacter bartlettii and Ruminococcus bromii and:
one or several bacterial strain(s) able to convert primary substrates into acetate such as described here above; and/or
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Paraclostridium bifermentans and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter othiniensis and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Blautia_A wexlerae and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus Terrisporobacter glycolicus and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Terrisporobacter mayombei and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Eubacterium limosum and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Lactobacillus rhamnosus and Intestinibacter bartlettii and Ruminococcus bromii and Bifidobacterium adolescentis and iv)
one or several bacterial strain(s) able to convert primary substrates into succinate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae and/or Anaerobutyricum hallii, iii) Bacteroides xylanisolvens or Prevotella copri or a bacterium of the Acutalibacter genus, preferably having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto and iv):
one or several bacterial strain(s) able to convert primary substrates into lactate such as described here above; and/or
one or several bacterial strain(s) able to convert primary substrates into formate such as described here above; and/or
one or several bacterial strain(s) able to convert formate into acetate such as described here above; and/or
one or several bacterial strain(s) able to convert primary substrates into acetate such as described here above; and/or
one or several bacterial strain(s) able to convert succinate into propionate such as described here above.

In some embodiments, the consortium according to the invention comprises or consists of i) Agathobacter rectalis, ii) Anaerostipes caccae o and/or r Anaerobutyricum hallii, iii) Phascolarctobacterium faecium and iv)
one or several bacterial strain(s) able to convert primary substrates into lactate such as described here above; and/or
one or several bacterial strain(s) able to convert formate into acetate such as described here above; and/or
one or several bacterial strain(s) able to convert primary substrates into acetate such as described here above; and/or
one or several bacterial strain(s) able to convert primary substrates into succinate such as described here above; and/or
one or several bacterial strain(s) able to convert primary substrates into formate such as described here above.

In some embodiments, the consortium according to the invention comprises or consists of i) Agathobacter rectalis, ii) Anaerostipes caccae and/or Anaerobutyricum hallii, iii) Phascolarctobacterium faecium, iv) Bacteroides xylanisolvens or Prevotella copri or a bacterium of the Acutalibacter genus, preferably having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto and v):
one or several bacterial strain(s) able to convert primary substrates into lactate such as described here above; and/or
one or several bacterial strain(s) able to convert primary substrates into formate such as described here above; and/or
one or several bacterial strain(s) able to convert formate into acetate such as described here above; and/or
one or several bacterial strain(s) able to convert primary substrates into acetate such as described here above.

In a very particular embodiment, the consortium according to the invention comprises or consists of i) Agathobacter rectalis, ii) Anaerostipes caccae and/or Anaerobutyricum hallii and iii) Ruminococcus bromii; Bifidobacterium adolescentis; Lactobacillus rhamnosus; Blautia hydrogenotrophica; Phascolarctobacterium faecium and a bacterium selected from the group consisting of Bacteroides xylanisolvens, Prevotella copri, a bacterium of the Acutalibacter genus, preferably having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto.

In a very particular embodiment, the consortium according to the invention comprises or consists of i) Agathobacter rectalis, ii) Anaerostipes caccae and/or Anaerobutyricum hallii and iii) Ruminococcus bromii; Bifidobacterium adolescentis; Lactobacillus rhamnosus; Blautia hydrogenotrophica; Phascolarctobacterium faecium and a bacterium of the Acutalibacter genus, preferably having a DNA genome sequence as set forth in SEQ ID NO: 1 and/or having a 16RNA sequence as set forth in SEQ ID NO: 9.

In a very particular embodiment, the consortium according to the invention comprises or consists of i) Agathobacter rectalis, ii) Anaerostipes caccae or Anaerobutyricum hallii and iii) Ruminococcus bromii; Bifidobacterium adolescentis; Lactobacillus rhamnosus; Blautia hydrogenotrophica; Phascolarctobacterium faecium and a bacterium selected from the group consisting of Bacteroides xylanisolvens, Prevotella copri, a bacterium of the Acutalibacter genus, preferably having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto.

In a very particular embodiment, the consortium according to the invention comprises or consists of i) Agathobacter rectalis, ii) Anaerostipes caccae or Anaerobutyricum hallii and iii) Ruminococcus bromii; Bifidobacterium adolescentis; Lactobacillus rhamnosus; Blautia hydrogenotrophica; Phascolarctobacterium faecium and a bacterium of the Acutalibacter genus, preferably having a DNA genome sequence as set forth in SEQ ID NO: 1 and/or having a 16RNA sequence as set forth in SEQ ID NO: 9.

Preferably, the bacteria consortium of the invention comprises or essentially consists of:
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and Prevotella copri; or
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Blautia hydrogenotrophica; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Blautia hydrogenotrophica; Phascolarctobacterium faecium and Prevotella copri; or
Agathobacter rectalis; Anaerobutyricum hallii; Ruminococcus bromii; Bifidobacterium adolescentis; Lactobacillus rhamnosus; Blautia hydrogenotrophica; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Blautia hydrogenotrophica; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Blautia hydrogenotrophica; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Blautia hydrogenotrophica; Phascolarctobacterium faecium and a bacterium having a DNA genome sequence having at least 90%, preferably at least 95%, even more preferably at least 99% sequence identity with SEQ ID NO: 1.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and Bacteroides xylanisolvens, and a bacterium selected from the group consisting of Blautia hydrogenotrophica, Paraclostridium bifermentans, Terrisporobacter glycolicus, Terrisporobacter mayombei, Intestinibacter bartlettii, Eubacterium callanderi and Eubacterium limosum, preferably from the group consisting of Paraclostridium bifermentans, Terrisporobacter glycolicus, Terrisporobacter mayombei, Intestinibacter bartlettii, Eubacterium callanderi and Eubacterium limosum, preferably Intestinibacter bartlettii.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and Bacteroides xylanisolvens, and a bacterium selected from the group consisting of Blautia hydrogenotrophica, Terrisporobacter glycolicus, Blautia_A wexlerae, Blautia_A luti Terrisporobacter othiniensis, Terrisporobacter mayombei, Intestinibacter bartlettii, Eubacterium callanderi and Eubacterium limosum, preferably from the group consisting of Blautia hydrogenotrophica, Blautia_A wexlerae, Blautia_A luti, Terrisporobacter glycolicus, Terrisporobacter mayombei and Terrisporobacter othiniensis, even more preferably from the group consisting of Blautia hydrogenotrophica, Blautia_A wexlerae, Terrisporobacter and Terrisporobacter othiniensis.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and Bacteroides xylanisolvens, and a bacterium selected from the group consisting of Blautia hydrogenotrophica, Terrisporobacter glycolicus, Blautia_A wexlerae, Blautia_A luti Terrisporobacter othiniensis, Terrisporobacter mayombei, Intestinibacter bartlettii, Eubacterium callanderi and Eubacterium limosum, preferably from the group consisting of Blautia hydrogenotrophica, Blautia_A wexlerae, Blautia_A luti, Terrisporobacter glycolicus, Terrisporobacter mayombei and Terrisporobacter othiniensis, even more preferably from the group consisting of Blautia hydrogenotrophica, Blautia_A wexlerae, Terrisporobacter and Terrisporobacter othiniensis.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and Prevotella copri, and a bacterium selected from the group consisting of Blautia hydrogenotrophica, Paraclostridium bifermentans, Terrisporobacter glycolicus, Terrisporobacter mayombei, Intestinibacter bartlettii, Eubacterium callanderi and Eubacterium limosum, preferably from the group consisting of Paraclostridium bifermentans, Terrisporobacter glycolicus, Terrisporobacter mayombei, Intestinibacter bartlettii, Eubacterium callanderi and Eubacterium limosum, preferably Intestinibacter bartlettii.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and Prevotella copri, and a bacterium selected from the group consisting of Blautia hydrogenotrophica, Terrisporobacter glycolicus, Terrisporobacter mayombei, Intestinibacter bartlettii, Eubacterium callanderi and Eubacterium limosum, preferably from the group consisting of Blautia hydrogenotrophica, Blautia_A wexlerae, Blautia_A luti, Terrisporobacter glycolicus, Terrisporobacter mayombei and Terrisporobacter othiniensis, even more preferably from the group consisting of Blautia hydrogenotrophica, Blautia_A wexlerae, Terrisporobacter and Terrisporobacter othiniensis.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and Prevotella copri, and a bacterium selected from the group consisting of Blautia hydrogenotrophica, Terrisporobacter glycolicus, Blautia_A wexlerae, Blautia_A luti Terrisporobacter othiniensis, Terrisporobacter mayombei, Intestinibacter bartlettii, Eubacterium callanderi and Eubacterium limosum, preferably from the group consisting of Blautia hydrogenotrophica, Blautia_A wexlerae, Blautia_A luti, Terrisporobacter glycolicus, Terrisporobacter mayombei and Terrisporobacter othiniensis, even more preferably from the group consisting of Blautia hydrogenotrophica, Blautia_A wexlerae, Terrisporobacter and Terrisporobacter othiniensis.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and a bacterium Acutalibacter nov. genus having a DNA genome sequence having 90%, 95% or 99% sequence identity with SEQ ID NO: 1, and a bacterium selected from the group consisting of Blautia hydrogenotrophica, Paraclostridium bifermentans, Terrisporobacter glycolicus, Terrisporobacter mayombei, Intestinibacter bartlettii, Eubacterium callanderi and Eubacterium limosum, preferably from the group consisting of Paraclostridium bifermentans, Terrisporobacter glycolicus, Terrisporobacter mayombei, Intestinibacter bartlettii, Eubacterium callanderi and Eubacterium limosum, preferably Intestinibacter bartlettii.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and a bacterium Acutalibacter nov. genus having a DNA genome sequence having 90%, 95% or 99% sequence identity with SEQ ID NO: 1, and a bacterium selected from the group consisting of Blautia hydrogenotrophica, Terrisporobacter glycolicus, Blautia_A wexlerae, Blautia_A luti Terrisporobacter othiniensis, Terrisporobacter mayombei, Intestinibacter bartlettii, Eubacterium callanderi and Eubacterium limosum, preferably from the group consisting of Blautia hydrogenotrophica, Blautia_A wexlerae, Blautia_A luti, Terrisporobacter glycolicus, Terrisporobacter mayombei and Terrisporobacter othiniensis, even more preferably from the group consisting of Blautia hydrogenotrophica, Blautia_A wexlerae, Terrisporobacter and Terrisporobacter othiniensis.

In some aspects, the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and a bacterium Acutalibacter nov. genus having a DNA genome sequence having 90%, 95% or 99% sequence identity with SEQ ID NO: 1, and a bacterium selected from the group consisting of Blautia hydrogenotrophica, Terrisporobacter glycolicus, Blautia_A wexlerae, Blautia_A luti Terrisporobacter othiniensis, Terrisporobacter mayombei, Intestinibacter bartlettii, Eubacterium callanderi and Eubacterium limosum, preferably from the group consisting of Blautia hydrogenotrophica, Blautia_A wexlerae, Blautia_A luti, Terrisporobacter glycolicus, Terrisporobacter mayombei and Terrisporobacter othiniensis, even more preferably from the group consisting of Blautia hydrogenotrophica, Blautia_A wexlerae, Terrisporobacter and Terrisporobacter othiniensis.

In some, the bacteria consortium of the invention comprises or essentially consists of:
Agathobacter rectalis; Anaerostipes Caccae; Ruminococcus bromii; Bifidobacterium adolescentis; Lactobacillus rhamnosus; Paraclostridium bifermentans; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Paraclostridium bifermentans; Phascolarctobacterium faecium and Prevotella copri; or
Agathobacter rectalis; Anaerostipes Caccae; Ruminococcus bromii; Bifidobacterium adolescentis; Lactobacillus rhamnosus; Paraclostridium bifermentans; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Paraclostridium bifermentans; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Paraclostridium bifermentans; Phascolarctobacterium faecium and Prevotella copri; or
Agathobacter rectalis; Anaerobutyricum hallii; Ruminococcus bromii; Bifidobacterium adolescentis; Lactobacillus rhamnosus; Paraclostridium bifermentans; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Paraclostridium bifermentans; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Paraclostridium bifermentans; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Paraclostridium bifermentans; Phascolarctobacterium faecium and a bacterium having a DNA genome sequence having at least 90%, preferably at least 95%, even more preferably at least 99% sequence identity with SEQ ID NO: 1.

In some, the bacteria consortium of the invention comprises or essentially consists of:
Agathobacter rectalis; Anaerostipes Caccae; Ruminococcus bromii; Bifidobacterium adolescentis; Lactobacillus rhamnosus; Terrisporobacter othiniensis; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Terrisporobacter othiniensis; Phascolarctobacterium faecium and Prevotella copri; or
Agathobacter rectalis; Anaerostipes Caccae; Ruminococcus bromii; Bifidobacterium adolescentis; Lactobacillus rhamnosus; Terrisporobacter othiniensis; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Terrisporobacter othiniensis; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Terrisporobacter othiniensis; Phascolarctobacterium faecium and Prevotella copri; or
Agathobacter rectalis; Anaerobutyricum hallii; Ruminococcus bromii; Bifidobacterium adolescentis; Lactobacillus rhamnosus; Terrisporobacter othiniensis; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Terrisporobacter othiniensis; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Terrisporobacter othiniensis; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Terrisporobacter othiniensis; Phascolarctobacterium faecium and a bacterium having a DNA genome sequence having at least 90%, preferably at least 95%, even more preferably at least 99% sequence identity with SEQ ID NO: 1.

In some, the bacteria consortium of the invention comprises or essentially consists of:
Agathobacter rectalis; Anaerostipes Caccae; Ruminococcus bromii; Bifidobacterium adolescentis; Lactobacillus rhamnosus; Blautia_A wexlerae; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Blautia_A wexlerae; Phascolarctobacterium faecium and Prevotella copri; or
Agathobacter rectalis; Anaerostipes Caccae; Ruminococcus bromii; Bifidobacterium adolescentis; Lactobacillus rhamnosus; Blautia_A wexlerae; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Blautia_A wexlerae; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Blautia_A wexlerae; Phascolarctobacterium faecium and Prevotella copri; or
Agathobacter rectalis; Anaerobutyricum hallii; Ruminococcus bromii; Bifidobacterium adolescentis; Lactobacillus rhamnosus; Blautia_A wexlerae; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Blautia_A wexlerae; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Blautia_A wexlerae; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Blautia_A wexlerae; Phascolarctobacterium faecium and a bacterium having a DNA genome sequence having at least 90%, preferably at least 95%, even more preferably at least 99% sequence identity with SEQ ID NO: 1.

In some, the bacteria consortium of the invention comprises or essentially consists of:
Agathobacter rectalis; Anaerostipes Caccae; Ruminococcus bromii; Bifidobacterium adolescentis; Lactobacillus rhamnosus; Terrisporobacter glycolicus; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Terrisporobacter glycolicus; Phascolarctobacterium faecium and Prevotella copri; or
Agathobacter rectalis; Anaerostipes Caccae; Ruminococcus bromii; Bifidobacterium adolescentis;
Lactobacillus rhamnosus; Terrisporobacter glycolicus; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Terrisporobacter glycolicus; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Terrisporobacter glycolicus; Phascolarctobacterium faecium and Prevotella copri; or
Agathobacter rectalis; Anaerobutyricum hallii; Ruminococcus bromii; Bifidobacterium adolescentis; Lactobacillus rhamnosus; Terrisporobacter glycolicus; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Terrisporobacter glycolicus; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Terrisporobacter glycolicus; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Terrisporobacter glycolicus; Phascolarctobacterium faecium and a bacterium having a DNA genome sequence having at least 90%, preferably at least 95%, even more preferably at least 99% sequence identity with SEQ ID NO: 1.

In some, the bacteria consortium of the invention comprises or essentially consists of:
Agathobacter rectalis; Anaerostipes Caccae; Ruminococcus bromii; Bifidobacterium adolescentis; Lactobacillus rhamnosus; Terrisporobacter mayombei; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Terrisporobacter mayombei; Phascolarctobacterium faecium and Prevotella copri; or
Agathobacter rectalis; Anaerostipes Caccae; Ruminococcus bromii; Bifidobacterium adolescentis; Lactobacillus rhamnosus; Terrisporobacter mayombei; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Terrisporobacter mayombei; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Terrisporobacter mayombei; Phascolarctobacterium faecium and Prevotella copri; or
Agathobacter rectalis; Anaerobutyricum hallii; Ruminococcus bromii; Bifidobacterium adolescentis; Lactobacillus rhamnosus; Terrisporobacter mayombei; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Terrisporobacter mayombei; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Terrisporobacter mayombei; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Terrisporobacter mayombei; Phascolarctobacterium faecium and a bacterium having a DNA genome sequence having at least 90%, preferably at least 95%, even more preferably at least 99% sequence identity with SEQ ID NO: 1.

In some, the bacteria consortium of the invention comprises or essentially consists of:
Agathobacter rectalis; Anaerostipes Caccae; Ruminococcus bromii; Bifidobacterium adolescentis; Lactobacillus rhamnosus; Intestinibacter bartlettii; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Intestinibacter bartlettii; Phascolarctobacterium faecium and Prevotella copri; or
Agathobacter rectalis; Anaerostipes Caccae; Ruminococcus bromii; Bifidobacterium adolescentis; Lactobacillus rhamnosus; Intestinibacter bartlettii; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Intestinibacter bartlettii; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Intestinibacter bartlettii; Phascolarctobacterium faecium and Prevotella copri; or
Agathobacter rectalis; Anaerobutyricum hallii; Ruminococcus bromii; Bifidobacterium adolescentis; Lactobacillus rhamnosus; Intestinibacter bartlettii; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Intestinibacter bartlettii; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Intestinibacter bartlettii; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Intestinibacter bartlettii; Phascolarctobacterium faecium and a bacterium having a DNA genome sequence having at least 90%, preferably at least 95%, even more preferably at least 99% sequence identity with SEQ ID NO: 1.

In some, the bacteria consortium of the invention comprises or essentially consists of:
Agathobacter rectalis; Anaerostipes Caccae; Ruminococcus bromii; Bifidobacterium adolescentis; Lactobacillus rhamnosus; Eubacterium limosum; Phascolarctobacterium faecium and Bacteroides xylanisolvens; or
Agathobacter rectalis; Anaerostipes Caccae; Ruminococcus bromii; Bifidobacterium adolescentis; Lactobacillus rhamnosus; Eubacterium limosum; Phascolarctobacterium faecium and Prevotella copri; or
Agathobacter rectalis; Anaerostipes Caccae; Ruminococcus bromii; Bifidobacterium adolescentis; Lactobacillus rhamnosus; Eubacterium limosum; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerobutyricum hallii; Ruminococcus bromii; Bifidobacterium adolescentis; Lactobacillus rhamnosus; Eubacterium limosum; Phascolarctobacterium faecium and Bacteroides xylanisolvens; or
Agathobacter rectalis; Anaerobutyricum hallii; Ruminococcus bromii; Bifidobacterium adolescentis; Lactobacillus rhamnosus; Eubacterium limosum; Phascolarctobacterium faecium and Prevotella copri; or
Agathobacter rectalis; Anaerobutyricum hallii; Ruminococcus bromii; Bifidobacterium adolescentis; Lactobacillus rhamnosus; Eubacterium limosum; Phascolarctobacterium faecium and a bacterium having a DNA genome sequence having at least 90%, preferably at least 95%, even more preferably at least 99% sequence identity with SEQ ID NO: 1; or
Agathobacter rectalis; Anaerostipes Caccae; Anaerobutyricum hallii; Ruminococcus bromii; Bifidobacterium adolescentis; Lactobacillus rhamnosus; Eubacterium limosum; Phascolarctobacterium faecium and Bacteroides xylanisolvens;
Agathobacter rectalis; Anaerostipes Caccae; Anaerobutyricum hallii; Ruminococcus bromii; Bifidobacterium adolescentis; Lactobacillus rhamnosus; Eubacterium limosum; Phascolarctobacterium faecium and Prevotella copri; or
Agathobacter rectalis; Anaerostipes Caccae; Anaerobutyricum hallii; Ruminococcus bromii; Bifidobacterium adolescentis; Lactobacillus rhamnosus; Eubacterium limosum; Phascolarctobacterium faecium and a bacterium having a DNA genome sequence having at least 90%, preferably at least 95%, even more preferably at least 99% sequence identity with SEQ ID NO: 1.

Preferably, the consortium according to the invention comprises or consists of Agathobacter rectalis and Anaerostipes caccae; Ruminococcus bromii; Bifidobacterium adolescentis; Lactobacillus rhamnosus; Blautia hydrogenotrophica; Phascolarctobacterium faecium and Bacteroides xylanisolvens.

Alternatively, the consortium according to the invention comprises or consists of Agathobacter rectalis and Anaerostipes caccae; Ruminococcus bromii; Bifidobacterium adolescentis; Lactobacillus rhamnosus; Blautia hydrogenotrophica; Phascolarctobacterium faecium and Prevotella copri.

In particular, such consortia do not comprise a bacterial strain of the genus Clostridium.

Preferably, such consortia do not comprise:
one or more bacterial strain(s) selected from the species Anaerotignum nov sp., Clostridium_E sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella; even more preferably wherein the consortium does not comprise a bacterial strain able to convert lactate into propionate; and/or
one or more bacterial strain(s) selected from the species Anaerotignum lactatifermentans (e.g., DSM 14214), Anaerotignum sp000436415, Bacteroides fragilis (e.g., ATCC 25285, DSM 2151, JCM 11019), Phocaeicola vulgatus (e.g., ATCC 8482, DSM 1447, JCM 5826) and Veillonella ratti (e.g., ATCC 17746, DSM 20736, JCM 6512); preferably from Bacteroides fragilis (e.g., ATCC 25285, DSM 2151, JCM 11019) and Phocaeicola vulgatus (e.g., ATCC 8482, DSM 1447, JCM 5826); preferably selected from the genera Anaerotignum, Bacteroides, Phocaeicola and Veillonella; even more preferably wherein the consortium does not comprise a bacterial strain able to convert primary substrates into propionate.

In some aspects, Agathobacter rectalis can be replaced in the consortium by any bacterium or combination of bacteria able to perform pathways B2 and A4 such as described herein.

In some aspects, Anaerostipes caccae and/or Anaerobutyricum hallii can be replaced in the consortium by any bacterium or combination of bacteria able to perform pathways A3, B2 and B3 such as described herein.

In some embodiments, the consortium according to the invention does not comprise Eubacterium limosum and/or Faecalibacterium prausnitzii. Preferably, the composition according to the invention does not comprise Eubacterium limosum, Faecalibacterium prausnitzii nor a bacterium from the genus Clostridium. Even more preferably, the consortium according to the invention does not comprise Eubacterium limosum, Faecalibacterium prausnitzii and/or one or more bacterial strain(s) selected from the species Anaerotignum nov sp., Clostridium sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella. Most preferably, the consortium according to the invention does not comprise Eubacterium limosum, F. prausnitzii, a bacterium from the genus Clostridium nor a bacterial strain able to convert lactate into propionate.

In any of the embodiments described herein, the genus Blautia can be replaced by the genus Eubacterium, Paraclostridium, Terrisporobacter or Intestinibacter, especially when the genera Blautia is included in the consortium for its capacity to perform B1 and/or C2 pathways, preferably B1 pathway.

In any of the embodiments described herein, the genus Blautia can be replaced by the genus Eubacterium, especially when the genera Blautia is included in the consortium for its capacity to perform B1 and/or C2 pathways, preferably B1 pathway.

In any of the embodiments described herein, the genus Blautia can be replaced by the genus Paraclostridium, especially when the genera Blautia is included in the consortium for its capacity to perform B1 and/or C2 pathways, preferably B1 pathway.

In any of the embodiments described herein, the genus Blautia can be replaced by the genus Terrisporobacter, especially when the genera Blautia is included in the consortium for its capacity to perform B1 and/or C2 pathways, preferably B1 pathway.

In any of the embodiments described herein, the genus Blautia can be replaced by the genus Intestinibacter, especially when the genera Blautia is included in the consortium for its capacity to perform B1 and/or C2 pathways, preferably B1 pathway.

In any of the embodiments described herein, the specie Blautia hydrogenotrophica can be replaced by the species Eubacterium limosum, Paraclostridium bifermentans, Terrisporobacter glycolicus, Intestinibacter orbartlettii or Terrisporobacter mayombei, especially when Blautia hydrogenotrophica is included in the consortium for its capacity to perform B1 and/or C2 pathways, preferably B1 pathway.

Particularly, in any of the embodiments described herein, the specie Blautia hydrogenotrophica can be replaced by the species Eubacterium limosum Terrisporobacter glycolicus, Terrisporobacter othiniensis, Blautia_A wexlerae, Blautia_A luti, Intestinibacter orbartlettii or Terrisporobacter mayombei, especially when Blautia hydrogenotrophica is included in the consortium for its capacity to perform B1 and/or C2 pathways, preferably B1 pathway.

Particularly, in any of the embodiments described herein, the specie Blautia hydrogenotrophica can be replaced by the species Terrisporobacter othiniensis or Blautia_A wexlerae, especially when Blautia hydrogenotrophica is included in the consortium for its capacity to perform B1 and/or C2 pathways, preferably B1 pathway.

In any of the embodiments described herein, Blautia hydrogenotrophica can be replaced by Eubacterium limosum, especially when Blautia hydrogenotrophica is included in the consortium for its capacity to perform B1 and/or C2 pathways, preferably B1 pathway.

In any of the embodiments described herein, Blautia hydrogenotrophica can be replaced by Paraclostridium bifermentans, especially when Blautia hydrogenotrophica is included in the consortium for its capacity to perform B1 and/or C2 pathways, preferably B1 pathway.

In any of the embodiments described herein, Blautia hydrogenotrophica can be replaced by Terrisporobacter othiniensis, especially when Blautia hydrogenotrophica is included in the consortium for its capacity to perform B1 and/or C2 pathways, preferably B1 pathway.

In any of the embodiments described herein, Blautia hydrogenotrophica can be replaced by Blautia_A luti, especially when Blautia hydrogenotrophica is included in the consortium for its capacity to perform B1 and/or C2 pathways, preferably B1 pathway.

In any of the embodiments described herein, Blautia hydrogenotrophica can be replaced by Blautia_A wexlerae, especially when Blautia hydrogenotrophica is included in the consortium for its capacity to perform B1 and/or C2 pathways, preferably B1 pathway.

In any of the embodiments described herein, Blautia hydrogenotrophica can be replaced by Terrisporobacter glycolicus, especially when Blautia hydrogenotrophica is included in the consortium for its capacity to perform B1 and/or C2 pathways, preferably B1 pathway.

In any of the embodiments described herein, Blautia hydrogenotrophica can be replaced by Intestinibacter orbartlettii, especially when Blautia hydrogenotrophica is included in the consortium for its capacity to perform B1 and/or C2 pathways, preferably B1 pathway.

In any of the embodiments described herein, Blautia hydrogenotrophica can be replaced by Terrisporobacter mayombei, especially when Blautia hydrogenotrophica is included in the consortium for its capacity to perform B1 and/or C2 pathways, preferably B1 pathway.

In any of the embodiments described herein, Blautia_A wexlerae is preferably a bacterium having a 16SRNA sequence as set forth in SEQ ID NO: 17 and any variant thereof having at least 97%, 98% or 99% sequence identity thereto, preferably having a 16SRNA sequence as set forth in SEQ ID NO: 17.

### Aspects of the Invention Directed to a Formate-Module

In one aspect, the invention is directed to a composition comprising a bacterial formate-module. Bacteria of the formate module perform i) pathway B1, and/or pathway B1 and C2.

Preferably, the invention is directed to a composition comprising a bacterial formate-module ((i.e. one or several bacterial strain(s) able to convert formate to acetate) in particular such as described in the above section “Consortium of bacterial strains”; and
a lactate module (i.e. one or several bacterial strain(s) able to convert primary substrates into lactate and one or several bacterial strain(s) able to convert lactate into butyrate and/or propionate); in particular such as described in the above section “Consortium of bacterial strains” and/or
   a succinate module (i.e. one or several bacterial strain(s) able to convert primary substrates into succinate; and/or one or several bacterial strain(s) able to convert succinate into propionate, in particular such as described in the above section “Consortium of bacterial strains”.

In one aspect, the invention is directed to a composition comprising a bacteria consortium of anaerobic bacterial strains, wherein said consortium comprises a bacterium of the genus Paraclostridium, preferably of specie Paraclostridium bifermentans, and comprises no more than 15 different bacterial strains, and comprises one or several bacterial strain(s) able to convert primary substrates into formate, lactate, succinate, acetate, butyrate and/or propionate, wherein the primary substrates are selected from the group consisting of sugars, starches, fibers and proteins and any combination thereof.

Preferably, the composition a bacterium of the genus Paraclostridium, preferably of specie Paraclostridium bifermentans, comprises one or several bacterial strain(s) able to convert primary substrates into formate (A1); for example such bacterial strain(s) may be selected from the genera Anaerobutyricum, Bacteroides, Collinsella, Coprococcus, Dorea, Eryispelatoclostridium, Eubacterium, Faealibacterium, Lachnospira, Longicatena, Ruminococcus and Sellimonas or from the species Anaerobutyricum hallii, Bacteroides clarus, Collinsella aerofaciens, Coprococcus eutactus, formicigenerans, Dorea Dorea longicatena, Erysipelatoclostridium ramosum, Eubacterium ramulus, Faecalibacterium prausnitzii, Lachnospira eligens, Longicatena sp. (in particular having a 16SRNA sequence such as set forth in SEQ ID NO: 11 or any variant thereof having at least 97%, 98% or 99% sequence identity thereto), Ruminococcus bromii, Sellimonas intestinalis and any combination thereof. Particularly preferred is a (A1) converter of the genus Ruminococcus and of the species Ruminococcus bromii.

In some aspects, the composition comprises a formate module comprising a bacterium of the genus Paraclostridium, preferably of specie Paraclostridium bifermentans and:
a lactate module (i.e. one or several bacterial strain(s) able to convert primary substrates into lactate and one or several bacterial strain(s) able to convert lactate into butyrate and/or propionate); in particular such as described in section “Consortium of bacterial strains” and/or
a succinate module (i.e. one or several bacterial strain(s) able to convert primary substrates into succinate; and/or one or several bacterial strain(s) able to convert succinate into propionate, in particular such as described in section “Consortium of bacterial strains”.

In one aspect, the invention is directed to a composition comprising a bacteria consortium of anaerobic bacterial strains, wherein said consortium comprises a bacterium of the genus Paraclostridium, preferably of species Paraclostridium bifermentans, and comprises:
one or several bacterial strain(s) performing pathway A1, in particular of a bacterial genus or specie such as described hereabove;
one or several bacterial strain(s) performing pathway A2, in particular of a bacterial genus or specie such as described hereabove;
one or several bacterial strain(s) performing pathway A3, in particular of a bacterial genus or specie such as described hereabove;
one or several bacterial strain(s) performing pathway A4, in particular of a bacterial genus or specie such as described hereabove;
one or several bacterial strain(s) performing pathway A5, in particular of a bacterial genus or specie such as described hereabove;
one or several bacterial strain(s) performing pathway A6 in particular of a bacterial genus or specie such as described hereabove;
one or several bacterial strain(s) performing pathway B2 in particular of a bacterial genus or specie such as described hereabove;
one or several bacterial strain(s) performing pathway B3 in particular of a bacterial genus or specie such as described hereabove;
optionally one or several bacterial strain(s) performing pathway C1, in particular of a bacterial genus or specie such as described hereabove; and/or
optionally one or several bacterial strain(s) performing pathway C2, in particular a bacterium genus or specie such as described hereabove.

In one aspect, the invention is directed to a composition comprising a bacteria consortium of anaerobic bacterial strains, wherein said consortium comprises a bacterium of the genus Intestinibacter, preferably of species Intestinibacter bartlettii, and comprises no more than 15 different bacterial strains, and comprises one or several bacterial strain(s) able to convert primary substrates into formate, wherein the primary substrates are selected from the group consisting of sugars, starches, fibers and proteins and any combination thereof.

Preferably, the composition a bacterium of the genus Intestinibacter, preferably of species Intestinibacter bartlettii, comprises one or several bacterial strain(s) able to convert primary substrates into formate (A1); for example such bacterial strain(s) may be selected from the genera Anaerobutyricum, Bacteroides, Collinsella, Coprococcus, Dorea, Eryispelatoclostridium, Eubacterium, Faealibacterium, Lachnospira, Longicatena, Ruminococcus and Sellimonas or from the species Anaerobutyricum hallii, Bacteroides clarus, Collinsella aerofaciens, eutactus, Coprococcus Dorea formicigenerans, Dorea longicatena, Erysipelatoclostridium ramosum, Eubacterium ramulus, Faecalibacterium prausnitzii, Lachnospira eligens, Longicatena sp. (in particular having a 16SRNA sequence such as set forth in SEQ ID NO: 11 or any variant thereof having at least 97%, 98% or 99% sequence identity thereto), Ruminococcus bromii, Sellimonas intestinalis and any combination thereof. Particularly preferred is a (A1) converter of the genus Ruminococcus and of the species Ruminococcus bromii.

In some aspects, the composition comprises a formate module comprising a bacterium of the genus Intestinibacter, preferably of species Intestinibacter bartlettii, and:
a lactate module (i.e. one or several bacterial strain(s) able to convert primary substrates into lactate and one or several bacterial strain(s) able to convert lactate into butyrate and/or propionate); in particular such as described in the above section “Consortium of bacterial strains” and/or
a succinate module (i.e. one or several bacterial strain(s) able to convert primary substrates into succinate; and/or one or several bacterial strain(s) able to convert succinate into propionate, in particular such as described in section “Consortium of bacterial strains”.

In one aspect, the invention is directed to a composition comprising a bacteria consortium of anaerobic bacterial strains, wherein said consortium comprises a bacterium of the genus Intestinibacter, preferably of species Intestinibacter bartlettii, and comprises:
one or several bacterial strain(s) performing pathway A1, in particular of a bacterial genus or specie such as described hereabove;
one or several bacterial strain(s) performing pathway A2, in particular of a bacterial genus or specie such as described hereabove;
one or several bacterial strain(s) performing pathway A3, in particular of a bacterial genus or specie such as described hereabove;
one or several bacterial strain(s) performing pathway A4, in particular of a bacterial genus or specie such as described hereabove;
one or several bacterial strain(s) performing pathway A5, in particular of a bacterial genus or specie such as described hereabove;
one or several bacterial strain(s) performing pathway A6 in particular of a bacterial genus or specie such as described hereabove;
one or several bacterial strain(s) performing pathway B2 in particular of a bacterial genus or specie such as described hereabove;
one or several bacterial strain(s) performing pathway B3 in particular of a bacterial genus or specie such as described hereabove;
optionally one or several bacterial strain(s) performing pathway C1, in particular of a bacterial genus or specie such as described hereabove; and/or
optionally one or several bacterial strain(s) performing pathway C2, in particular a bacterium genus or specie such as described hereabove.

In one aspect, the invention is directed to a bacteria consortium of anaerobic bacterial strains, wherein said consortium comprises a bacterium of the genus Terrisprobacter, preferably of specie Terrisporobacter glycolicus or Terrisporobacter mayombei, and comprises no more than 15 different bacterial strains, and comprises one or several bacterial strain(s) able to convert primary substrates into formate, wherein the primary substrates are selected from the group consisting of sugars, starches, fibers and proteins and any combination thereof.

Preferably, the composition a bacterium of the genus Terrisprobacter, preferably of specie Terrisporobacter glycolicus comprises one or several bacterial strain(s) able to convert primary substrates into formate (A1); for example such bacterial strain(s) may be selected from the genera Anaerobutyricum, Bacteroides, Collinsella, Coprococcus, Dorea, Eryispelatoclostridium, Eubacterium, Faealibacterium, Lachnospira, Longicatena, Ruminococcus and Sellimonas or from the species Anaerobutyricum hallii, Bacteroides clarus, Collinsella aerofaciens, Coprococcus eutactus, formicigenerans, Dorea longicatena, Dorea Erysipelatoclostridium ramosum, Eubacterium ramulus, Faecalibacterium prausnitzii, Lachnospira eligens, Longicatena sp. (in particular having a 16SRNA sequence such as set forth in SEQ ID NO: 11 or any variant thereof having at least 97%, 98% or 99% sequence identity thereto), Ruminococcus bromii, Sellimonas intestinalis and any combination thereof. Particularly preferred is a (A1) converter of the genus Ruminococcus and of the species Ruminococcus bromii.

In some aspects, the composition comprises a formate module comprising a bacterium of the genus Terrisprobacter, preferably of specie Terrisporobacter glycolicus, and:
a lactate module (i.e. one or several bacterial strain(s) able to convert primary substrates into lactate and one or several bacterial strain(s) able to convert lactate into butyrate and/or propionate); in particular such as described in the above section “Consortium of bacterial strains” and/or
a succinate module (i.e. one or several bacterial strain(s) able to convert primary substrates into succinate; and/or one or several bacterial strain(s) able to convert succinate into propionate, in particular such as described in the above section “Consortium of bacterial strains”.

In one aspect, the invention is directed to a composition comprising a bacteria consortium of anaerobic bacterial strains, wherein said consortium comprises a bacterium of the genus Terrisprobacter, preferably of specie Terrisporobacter glycolicus and comprises:
one or several bacterial strain(s) performing pathway A1, in particular of a bacterial genus or specie such as described hereabove;
one or several bacterial strain(s) performing pathway A2, in particular of a bacterial genus or specie such as described hereabove;
one or several bacterial strain(s) performing pathway A3, in particular of a bacterial genus or specie such as described hereabove;
one or several bacterial strain(s) performing pathway A4, in particular of a bacterial genus or specie such as described hereabove;
one or several bacterial strain(s) performing pathway A5, in particular of a bacterial genus or specie such as described hereabove;
one or several bacterial strain(s) performing pathway A6 in particular of a bacterial genus or specie such as described hereabove;
one or several bacterial strain(s) performing pathway B2 in particular of a bacterial genus or specie such as described hereabove;
one or several bacterial strain(s) performing pathway B3 in particular of a bacterial genus or specie such as described hereabove;
optionally one or several bacterial strain(s) performing pathway C1, in particular of a bacterial genus or specie such as described hereabove; and/or
optionally one or several bacterial strain(s) performing pathway C2, in particular a bacterium genus or specie such as described hereabove.

Preferably, the composition a bacterium of the genus Terrisprobacter, preferably of specie Terrisporobacter mayombei comprises one or several bacterial strain(s) able to convert primary substrates into formate (A1); for example such bacterial strain(s) may be selected from the genera Anaerobutyricum, Bacteroides, Collinsella, Coprococcus, Dorea, Eryispelatoclostridium, Eubacterium, Faealibacterium, Lachnospira, Longicatena, Ruminococcus and Sellimonas or from the species Anaerobutyricum hallii, Bacteroides clarus, Collinsella aerofaciens, Coprococcus eutactus, Dorea formicigenerans, Dorea longicatena, Erysipelatoclostridium ramosum, Eubacterium ramulus, Faecalibacterium prausnitzii, Lachnospira eligens, Longicatena sp. (in particular having a 16SRNA sequence such as set forth in SEQ ID NO: 11 or any variant thereof having at least 97%, 98% or 99% sequence identity thereto), Ruminococcus bromii, Sellimonas intestinalis and any combination thereof. Particularly preferred is a (A1) converter of the genus Ruminococcus and of the species Ruminococcus bromii.

In some aspects, the composition comprises a formate module comprising a bacterium of the genus Terrisprobacter, preferably of specie Terrisporobacter mayombei, and:
a lactate module (i.e. one or several bacterial strain(s) able to convert primary substrates into lactate and one or several bacterial strain(s) able to convert lactate into butyrate and/or propionate); in particular such as described in the above section “Consortium of bacterial strains” and/or
a succinate module (i.e. one or several bacterial strain(s) able to convert primary substrates into succinate; and/or one or several bacterial strain(s) able to convert succinate into propionate, in particular such as described in the above section “Consortium of bacterial strains”.

In one aspect, the invention is directed to a composition comprising a bacteria consortium of anaerobic bacterial strains, wherein said consortium comprises a bacterium of the genus Terrisprobacter, preferably of specie Terrisporobacter mayombei and comprises:
one or several bacterial strain(s) performing pathway A1, in particular of a bacterial genus or specie such as described hereabove;
one or several bacterial strain(s) performing pathway A2, in particular of a bacterial genus or specie such as described hereabove;
one or several bacterial strain(s) performing pathway A3, in particular of a bacterial genus or specie such as described hereabove;
one or several bacterial strain(s) performing pathway A4, in particular of a bacterial genus or specie such as described hereabove;
one or several bacterial strain(s) performing pathway A5, in particular of a bacterial genus or specie such as described hereabove;
one or several bacterial strain(s) performing pathway A6 in particular of a bacterial genus or specie such as described hereabove;
one or several bacterial strain(s) performing pathway B2 in particular of a bacterial genus or specie such as described hereabove;
one or several bacterial strain(s) performing pathway B3 in particular of a bacterial genus or specie such as described hereabove;
1 optionally one or several bacterial strain(s) performing pathway C1, in particular of a bacterial genus or specie such as described hereabove; and/or
optionally one or several bacterial strain(s) performing pathway C2, in particular a bacterium genus or specie such as described hereabove.

Preferably, the composition a bacterium of the genus Terrisprobacter, preferably of specie Terrisporobacter othiniensis comprises one or several bacterial strain(s) able to convert primary substrates into formate (A1); for example such bacterial strain(s) may be selected from the genera Anaerobutyricum, Bacteroides, Collinsella, Coprococcus, Dorea, Eryispelatoclostridium, Eubacterium, Faealibacterium, Lachnospira, Longicatena, Ruminococcus and Sellimonas or from the species Anaerobutyricum hallii, Bacteroides clarus, Collinsella aerofaciens, Coprococcus eutactus, Dorea formicigenerans, Dorea longicatena, Erysipelatoclostridium ramosum, Eubacterium ramulus, Faecalibacterium prausnitzii, Lachnospira eligens, Longicatena sp. (in particular having a 16SRNA sequence such as set forth in SEQ ID NO: 11 or any variant thereof having at least 97%, 98% or 99% sequence identity thereto), Ruminococcus bromii, Sellimonas intestinalis and any combination thereof. Particularly preferred is a (A1) converter of the genus Ruminococcus and of the species Ruminococcus bromii.

In some aspects, the composition comprises a formate module comprising a bacterium of the genus Terrisprobacter, preferably of specie Terrisporobacter othiniensis, and:
a lactate module (i.e. one or several bacterial strain(s) able to convert primary substrates into lactate and one or several bacterial strain(s) able to convert lactate into butyrate and/or propionate); in particular such as described in the above section “Consortium of bacterial strains” and/or
a succinate module (i.e. one or several bacterial strain(s) able to convert primary substrates into succinate; and/or one or several bacterial strain(s) able to convert succinate into propionate, in particular such as described in the above section “Consortium of bacterial strains”.

In one aspect, the invention is directed to a composition comprising a bacteria consortium of anaerobic bacterial strains, wherein said consortium comprises a bacterium of the genus Terrisprobacter, preferably of specie Terrisporobacter othiniensis and comprises:
one or several bacterial strain(s) performing pathway A1, in particular of a bacterial genus or specie such as described hereabove;
one or several bacterial strain(s) performing pathway A2, in particular of a bacterial genus or specie such as described hereabove;
one or several bacterial strain(s) performing pathway A3, in particular of a bacterial genus or specie such as described hereabove;
one or several bacterial strain(s) performing pathway A4, in particular of a bacterial genus or specie such as described hereabove;
one or several bacterial strain(s) performing pathway A5, in particular of a bacterial genus or specie such as described hereabove;
one or several bacterial strain(s) performing pathway A6 in particular of a bacterial genus or specie such as described hereabove;
one or several bacterial strain(s) performing pathway B2 in particular of a bacterial genus or specie such as described hereabove;
one or several bacterial strain(s) performing pathway B3 in particular of a bacterial genus or specie such as described hereabove;
optionally one or several bacterial strain(s) performing pathway C1, in particular of a bacterial genus or specie such as described hereabove; and/or
optionally one or several bacterial strain(s) performing pathway C2, in particular a bacterium genus or specie such as described hereabove.

Preferably, the composition a bacterium of the genus Blautia, preferably of specie Blautia_A luti, Blautia hydrogenotrophica or Blautia_A wexlerae, comprises one or several bacterial strain(s) able to convert primary substrates into formate (A1); for example such bacterial strain(s) may be selected from the genera Anaerobutyricum, Bacteroides, Collinsella, Coprococcus, Dorea, Eryispelatoclostridium, Eubacterium, Faealibacterium, Lachnospira, Longicatena, Ruminococcus and Sellimonas or from the species Anaerobutyricum hallii, Bacteroides clarus, Collinsella aerofaciens, Coprococcus eutactus, Dorea formicigenerans, Dorea longicatena, Erysipelatoclostridium ramosum, Eubacterium ramulus, Faecalibacterium prausnitzii, Lachnospira eligens, Longicatena sp. (in particular having a 16SRNA sequence such as set forth in SEQ ID NO: 11 or any variant thereof having at least 97%, 98% or 99% sequence identity thereto), Ruminococcus bromii, Sellimonas intestinalis and any combination thereof. Particularly preferred is a (A1) converter of the genus Ruminococcus and of the species Ruminococcus bromii.

In some aspects, the composition comprises a formate module comprising a bacterium of the genus Blautia, preferably of specie Blautia_A luti, Blautia hydrogenotrophica or Blautia_A wexlerae, and:
a lactate module (i.e. one or several bacterial strain(s) able to convert primary substrates into lactate and one or several bacterial strain(s) able to convert lactate into butyrate and/or propionate); in particular such as described in the above section “Consortium of bacterial strains” and/or
a succinate module (i.e. one or several bacterial strain(s) able to convert primary substrates into succinate; and/or one or several bacterial strain(s) able to convert succinate into propionate, in particular such as described in the above section “Consortium of bacterial strains”.

In one aspect, the invention is directed to a composition comprising a bacteria consortium of anaerobic bacterial strains, wherein said consortium comprises a bacterium of the genus Blautia, preferably of specie Blautia_A luti, Blautia hydrogenotrophica or Blautia_A wexlerae, and comprises:
one or several bacterial strain(s) performing pathway A1, in particular of a bacterial genus or specie such as described hereabove;
one or several bacterial strain(s) performing pathway A2, in particular of a bacterial genus or specie such as described hereabove;
one or several bacterial strain(s) performing pathway A3, in particular of a bacterial genus or specie such as described hereabove;
one or several bacterial strain(s) performing pathway A4, in particular of a bacterial genus or specie such as described hereabove;
one or several bacterial strain(s) performing pathway A5, in particular of a bacterial genus or specie such as described hereabove;
one or several bacterial strain(s) performing pathway A6 in particular of a bacterial genus or specie such as described hereabove;
one or several bacterial strain(s) performing pathway B2 in particular of a bacterial genus or specie such as described hereabove;
one or several bacterial strain(s) performing pathway B3 in particular of a bacterial genus or specie such as described hereabove;
optionally one or several bacterial strain(s) performing pathway C1, in particular of a bacterial genus or specie such as described hereabove; and/or
optionally one or several bacterial strain(s) performing pathway C2, in particular a bacterium genus or specie such as described hereabove.

The formate module may in particular include the following combinations of genera: Intestinibacter and Anaerobutyricum; Intestinibacter and Bacteroides; Intestinibacter and Collinsella; Intestinibacter and Coprococcus, Intestinibacter and Dorea, Intestinibacter and Eryispelatoclostridium, Intestinibacter and Eubacterium, Intestinibacter and Faealibacterium, Intestinibacter and Lachnospira, Intestinibacter and Longicatena, Intestinibacter and Ruminococcus; Intestinibacter and Sellimonas; Terrisporobacter and Anaerobutyricum; Terrisporobacter and Bacteroides; Terrisporobacter and Collinsella; Terrisporobacter and Coprococcus, Terrisporobacter and Dorea, Terrisporobacter and Eryispelatoclostridium, Terrisporobacter and Eubacterium, Terrisporobacter and Faealibacterium, Terrisporobacter and Lachnospira, Terrisporobacter and Longicatena, Terrisporobacter and Ruminococcus; Terrisporobacter and Sellimonas; Paraclostridium and Anaerobutyricum; Paraclostridium and Bacteroides; Paraclostridium and Collinsella; Paraclostridium and Coprococcus, Paraclostridium and Dorea, Paraclostridium and Eryispelatoclostridium, Paraclostridium and Eubacterium, Paraclostridium and Faealibacterium, Paraclostridium and Lachnospira, Paraclostridium and Longicatena, Paraclostridium and Ruminococcus; Paraclostridium and Sellimonas. Preferred are the combinations of Intestinibacter and Ruminococcus, Terrisporobacter and Ruminococcus, Paraclostridium and Ruminococcus.

In some aspects, the composition comprises a formate module comprising combinations of genera such as described above and:
a lactate module (i.e. one or several bacterial strain(s) able to convert primary substrates into lactate and one or several bacterial strain(s) able to convert lactate into butyrate and/or propionate); in particular such as described in the above section “Consortium of bacterial strains” and/or
a succinate module (i.e. one or several bacterial strain(s) able to convert primary substrates into succinate; and/or one or several bacterial strain(s) able to convert succinate into propionate, in particular such as described in the above section “Consortium of bacterial strains”.

### Compositions

In a further aspect, the present invention relates to a composition comprising any of the above-described consortia.

The composition of the invention may be a pharmaceutical composition, a food composition or a food supplement.

In preferred embodiments, the composition of the invention is a pharmaceutical composition comprising a consortium of bacterial strains such as disclosed herein and optionally a pharmaceutically acceptable carrier or excipient.

Typically, the composition according to the invention comprises at least 10⁴, at least 10⁵, at least 10⁶, preferably at least 10⁷ bacterial cells per ml or g for each bacterial strain and each of the bacterial strains has a viability over 10%, 20%, 30%, 40%, 50%, preferably over 70%.

As used herein, the term “pharmaceutically acceptable” refer to molecular entities and compositions that do not produce an adverse, allergic or other untoward reaction when administered to a mammal, especially a human, as appropriate. A pharmaceutically acceptable excipient refers to a non-toxic solid, semi-solid or liquid filler, diluent, encapsulating material or formulation auxiliary of any type. The pharmaceutically acceptable excipients that can be used in the composition according to the invention are well known to the skilled person and may vary according to the disease to be treated and the administration route.

The composition of the invention can be administered by any method suitable for depositing in the gastrointestinal tract, preferably the small intestine and/or the colon, of the subject to be treated.

Preferably, the composition of the invention is formulated either as a rectally administrated form or an orally ingestible form.

In an embodiment, the pharmaceutical composition is to be administered by oral route. For oral administration, the pharmaceutical composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants may also be necessary in the tablets to facilitate break-up of the tablet. Disintegrants include starches, clays, celluloses, algins, gums and crosslinked polymers. Moreover, lubricants and glidants may also be included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. Well-known thickening agents may also be added to compositions such as corn starch, agar, natural or synthetic gums, resins, methylcellulose, sodium carboxymethylcellulose, guar, xanthan and the like. Preservatives may also be included in the composition, including methylparaben, propylparaben, benzyl alcohol and ethylene diamine tetraacetate salts.

Preferably, for oral administration, the composition is in a gastro-resistant oral form allowing the active compounds contained in the composition, to pass the stomach and be released into the intestine. The material that can be used in enteric coatings includes, for example, alginic acid, cellulose acetate phthalate, plastics, waxes, shellac and fatty acids (e.g., stearic acid or palmitic acid).

In another embodiment, the pharmaceutical composition is to be administrated by rectal route. Suitable rectal-route forms include, but are not limited to, suppository and enema. In particular, the active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Compositions according to the invention may be formulated to release the active ingredients substantially immediately upon administration or at any predetermined time or time period after administration.

In others embodiments, the composition may be a food composition or a food supplement.

By “food composition” is meant any composition comprising food ingredients such as macronutrients, micronutrients, vitamins and/or minerals. The food composition may be intended for human or animal consumption and may be a liquid, paste or solid. Examples of food compositions include, but are not limited to dairy products such as cheese, butter, cream, yoghurt, fermented milk, ice cream, cooked products such as bread, biscuits and cakes, fruit products such as fruit juice, fruit compote or fruit paste, soy food products, starch-based food products, edible oil compositions, spreads, breakfast cereals, infant formula, food bars (e.g. cereal bars, breakfast bars, energy bars, nutrition bars), chewing gum, beverages, drinking supplements (powders to be added to a beverage).

As used herein, the term “food supplement” refers to any composition which is formulated and administered separately from other foods to complement the nutritional intakes of a subject, i.e., a human or an animal. This supplement may be in any suitable form well known to those skilled in the art, preferably in the form of dietetic food or oral supplementation.

In some embodiments, the composition according to the invention comprises a component selected from the group consisting of cryoprotecting media, in particular comprising glycerol; culture or dispersing media, in particular comprising peptone, yeast extract, monosaccharides, disaccharides, arabinogalactan, fructo-oligosaccharides, fibers, glycerol, soluble starch, resistant starch, xylan, minerals, co-factors, vitamins and reducing agents; aqueous gels; prebiotics and polymeric supports; and any combination thereof.

In some embodiments, the composition of the invention may particularly comprise one or several prebiotics. As used here, a “prebiotic” refers to an ingredient that can induce specific changes in both the composition and/or activity of the composition of bacterial strains and/or gastrointestinal microbiome that may confer benefits to the host. Preferably, the prebiotic can be degraded by the consortium of bacterial strains, and may increase its shelf life after administration to a patient. Examples of prebiotics include, but are not limited to, complex carbohydrates, polyphenols, amino acids, peptides, minerals, or other nutritional components promoting the survival of the bacterial strains of the consortium.

In some embodiments, the composition according to the invention is free of, or essentially free of succinate, formate and/or lactate.

In particular, the composition according to the invention comprises succinate in an amount of less than 5 mM, formate in an amount of less than 5 mM, and/or lactate in an amount of less than 5 mM.

In some embodiments, the composition according to the invention comprises propionate, acetate and/or butyrate.

In particular, the composition according to the invention comprises acetate in an amount of at least 10 mM, propionate in an amount of at least 2 mM, and/or butyrate in an amount of at least 2 mM.

In some embodiments, the composition of the invention comprises glycerol, in particular so as to enhance butyrate production.

### Uses of the Compositions

In a further aspect, the present invention also relates to the use of the consortium or composition as a medicament, especially in the treatment of a disorder or disease, in particular caused or resulted in intestinal dysbiosis.

As used herein, the term “medicament” refers to any substance or composition with curative or preventive properties against a disorder or disease.

Then invention also concerns a composition of the invention for use for treating a disease or a disorder, for improving the general health of a subject and/or for modifying the composition of the microbiome. It also relates to a composition for use for the manufacture of a medicament for treating a disease or disorder, for improving the general health of a subject and/or for modifying the composition of the microbiome. It also relates to the use of a composition as described herein for treating an intestinal dysbiosis or a disorder or a disease caused by or related to an intestinal dysbiosis.

In other aspects, the invention concerns a method for treating an intestinal dysbiosis or a disease or disorder caused by or related to an intestinal dysbiosis in a subject, wherein the method comprises administering a consortium of bacterial strains or composition of the invention to said subject.

The invention also concerns the use of a consortium of bacterial strains or composition of the invention for the manufacture of a medicament for the treatment of an intestinal dysbiosis or of a disease or disorder caused by or related to an intestinal dysbiosis.

Then, the invention also relates to a method for treating an intestinal dysbiosis or a disorder or a disease caused by an intestinal dysbiosis, for improving the general health of a subject and/or for modifying the composition of the microbiome, comprising administering a therapeutically effective amount of a composition of the invention to a subject in need thereof.

The term “dysbiosis” is known in the art and denotes the alteration of the microbiota in comparison to an healthy state. The microbiota state may be characterized by determining key markers, intermediate metabolites and end metabolites. Preferably, a healthy microbiota is characterized by the absence of intermediate metabolites. Accordingly, a state characterized by accumulation of intermediate metabolites is referred to as dysbiosis.

Preferably, a subject suffers from intestinal dysbiosis when succinate is present in the intestine in an amount of at least 5 mM, 10 mM, 25 mM or 50 mM and/or formate is present in the intestine in an amount of at least 5 mM, 10 mM, 25 mM or 50 mM, and/or lactate is present in the intestine in an amount of at least 5 mM, 10 mM, 25 mM or 50 mM.

As used herein, the term “subject” or “patient” refers to an animal such as dogs, cats, horses, cows, pigs, sheep and non-human primates or non-mammals such as poultry, preferably a mammal, more preferably a human, including adult and child.

The subject to be treated with the composition of the invention is an animal, preferably a mammal. In an embodiment, the subject is a domestic or farmed animal such as dogs, cats, cows, sheep, horses or rodents. In a preferred embodiment, the subject is a human, including adult, child, newborns and human at the prenatal stage. As used herein, the terms “subject”, “individual” and “patient” are interchangeable.

The term “treatment” refers to any act intended to ameliorate the health status of patients or subjects such as therapy, prevention, prophylaxis and retardation of a disease. It designates both a curative treatment and/or a prophylactic treatment of a disease. A curative treatment is defined as a treatment resulting in a cure or a treatment alleviating, improving and/or eliminating, reducing and/or stabilizing the symptoms of a disease or the suffering that it causes directly or indirectly. A prophylactic treatment comprises both a treatment resulting in the prevention of a disease and a treatment reducing and/or delaying the incidence of a disease or the risk of its occurrence. In certain embodiments, such term refers to the improvement or eradication of a disease, a disorder or symptoms associated with it. The term “treatment” includes the prevention of diseases described herein and the delay of progression of diseases described herein.

A “therapeutically effective amount” is an amount which, when administered to a subject, is sufficient to treat the targeted disease or disorder, or to produce the desired therapeutic effect. This amount may vary according to the disease and its severity, the physiological data and characteristics of the patient or subject to be treated (e.g., age, size, and weight), and the routes of administration.

Preferably, an “effective therapeutic amount” comprises 10³ to 10¹⁴ CFU (colony forming units), preferably 10⁶ to 10⁹ CFU of bacteria per ml or μg of the pharmaceutical composition.

In general, it is desirable to provide the subject with a dosage of the above composition or consortia in the range of from about 10³ to 10⁹ CFU/kg, preferably 10⁶ to 10⁹ CFU/kg (body weight of the subject), although a lower or higher dosage may be administered.

Alternatively, it can be desirable to provide the subject with a dosage of the composition of the invention in the range of from about 50 μg to 1 mg/kg, preferably 50 μg to 500 μg/kg, more preferably 50 μg to 250 μg/kg; even more preferably 50 μg to 100 μg/kg (body weight of the subject), although a lower or higher dosage may be administered.

In embodiments, a therapeutically efficient amount is preferably defined as the amount necessary for having an impact on intestinal inflammation or any symptom of the disease such as diarrhea, fever or pain.

The composition of the invention may be administered as a single dose or in multiple doses. In particular, depending on the subject's age or physiological condition, daily doses may be divided to facilitate administration, for example with one administration in the morning and another in the evening.

In some embodiments, the composition is to be administered regularly, preferably between every day and every month, more preferably between every day and every two weeks, more preferably between every day and every week. In some particular embodiments, the composition is to be administered every day.

The duration of treatment with the composition of the invention may be comprised between 1 day and several years, preferably between 1 day and one year, more preferably between 1 day and 6 months.

The pharmaceutical compositions may find use in a number of indications such as prophylaxis, treatment, prevention or delay of progression of a disease related to intestinal microbiome disbalance or associated with microbiota dysbiosis. It is generally accepted that dysbiosis originates from an ecological disbalance (e.g., based on trophism), characterized by disproportionate amounts or absence of bacteria strains in the microbiome of the patient which are essential for the establishment and/or maintenance of a healthy microbiome.

In some embodiments, the consortia and compositions disclosed herein are useful to treat an intestinal dysbiosis of a disease or disorder caused by or related to an intestinal dysbiosis.

In some embodiments, the disease or disorder is a dysbiosis, preferably an intestinal dysbiosis. Preferably, the intestinal dysbiosis is selected from the group consisting of an intestinal dysbiosis following antibiotics treatment, an intestinal dysbiosis following infection by vancomycin resistant enterococci, an intestinal dysbiosis following infection by carbapenem resistant enterococci, and an intestinal dysbiosis following post-infectious diarrhea.

Alternatively, the disease or disorder caused by or related to an intestinal dysbiosis is selected from the group consisting of inflammatory bowel disease, including ulcerative colitis and Crohn's disease; rheumatoid arthritis; multiple sclerosis; graft versus host disease; solid and liquid cancer, gastrointestinal cancer, colorectal cancer and acute myeloid leukemia. Alternatively, the disease or disorder caused by or related to an intestinal dysbiosis is selected from the group consisting of inflammatory bowel disease, ulcerative colitis, Crohn's disease; rheumatoid arthritis; multiple sclerosis; graft versus host disease; gastrointestinal cancer, colorectal cancer and acute myeloid leukemia.

In particular, the composition of the invention can be used to treat pathologies involving bacteria of the human microbiome, preferably the intestinal microbiome, such as inflammatory or auto-immune diseases, cancers, infections or brain disorders. Inflammatory bowel diseases have been in the focus of microbiota-based therapies for some time now (Caruso et al., Host-microbiota interactions in inflammatory bowel disease. Nat Rev Immunol 20, 411-426 (2020)). Indeed, some bacteria of the microbiome, without triggering any infection, can secrete molecules that will induce and/or enhance inflammatory or auto-immune diseases (De Luca, et al., The microbiome in autoimmune diseases, Clin. Exp. Immunol. 195 (1) p. 74-85 (2019)) or cancer development (Zitvogel et al., Cancer and the gut microbiota: an unexpected link, Sci. Transl. Med. 7, 271 (2015)). Recently, diseases such as rheumatoid arthritis (Maeda et al., Host-microbiota interactions in rheumatoid arthritis, Exp Mol Med, 51, 1-6 (2019)) and multiple sclerosis (Correale et al., The role of the gut microbiota in multiple sclerosis, Nat Rev Neurol 18, 544-558 (2022)) have increasingly come into focus in microbiome research. Therefore, disease or disorder to be treated by the composition according to the invention may, in particular, be a dysbiosis i) following antibiotics treatment, ii) following exacerbated immune response, or iii) associated with cancer.

In some embodiments, the disorder may be selected from the group of dysbiosis following antibiotics treatment, infection by vancomycin resistant enterococci (VRE), infection by carbapenem resistant enterococci (CRE), post-infectious diarrhea; inflammatory bowel disease (IBD), including ulcerative colitis (UC) and Crohn's disease (CD); rheumatoid arthritis (RA); multiple sclerosis (MS); graft versus host disease (GvHD); solid and liquid cancer, in particular gastrointestinal cancer, colorectal cancer (CRC), acute myeloid leukemia (AML); gastritis, colitis, gastroenteritis, gingivitis, nosocomial infection and Clostridium difficile infection (CDI). In a particular embodiment, the disease or disorder to be treated is selected from dysbiosis following antibiotics treatment, infection by vancomycin resistant enterococci, infection by carbapenem resistant enterococci; inflammatory bowel disease, including ulcerative colitis and Crohn's disease; rheumatoid arthritis; multiple sclerosis; graft versus host disease; solid and liquid cancer, in particular gastrointestinal cancer, colorectal cancer and acute myeloid leukemia.

The composition according to the invention may be used in combination with another therapy or treatment for intestinal dysbiosis or a disease or disorder related to intestinal dysbiosis.

For example, the composition or consortium of the invention may be combined with a dietary intervention, a treatment with a drug or a medicament candidate, or a prebiotic administration. The term “drug” includes synthesized pharmaceuticals but also biopharmaceuticals or biological medical products, such as vaccines, blood components, somatic cells, gene therapies, tissues, recombinant therapeutic proteins, etc. In particular, the term “drug” includes living medicines and preferably formulations comprising bacterial cells selected or engineered to possess therapeutic properties.

In some embodiments, the composition or consortium according to the invention is used in combination with an antibiotic agent, a prebiotic agent, an anticancer agent, selective nutrition, an agent to modulate transit time and/or for cleansing an in-situ microbiome space.

### Methods of Production

The invention also concerns a method for producing the consortium or composition such as disclosed herein in an appropriate culture medium.

The terms “dispersing medium”, “cultivation medium” and “culture medium” are used interchangeably herein and refer to a liquid or solid medium, preferably a liquid medium, in which one or several bacterial strains can be inoculated and/or cultivated. The composition of the culture medium depends on the nutritional requirements of the cultivated bacteria. This composition can be easily adjusted by the skilled person based on his general knowledge.

Typically, culture media include at least one carbon source (glycerol, glucose, galactose, maltose, lactose, sucrose, fructose, cellobiose), fibers (preferably pectin, arabinogalactan, beta-glucan, soluble starch, resistant starch, fructo-oligosaccharides, galacto-oligosaccharides, xylan, arabinoxylans, cellulose), proteins (preferably yeast extract, casein, skimmed milk, peptone), co-factors (short chain fatty acids, hemin, FeSO4), vitamins (preferably biotin, cobalamin, 4-aminobenzoic acid, folic acid, pyridoxamine hydrochloride), minerals (preferably sodium bicarbonate, potassium phosphate dibasic, potassium phosphate monobasic, sodium chloride, ammonium sulfate, magnesium sulfate, calcium chloride) and reducing agents (preferably cysteine, titanium (III)-citrate, yeast extract, sodium thioglycolate, dithiothreitol, sodium sulphide, hydrogen sulphite, ascorbate). Culture media can also comprise compound(s) preventing microbial contamination, e.g., antibiotics selected to be inefficient on desired bacteria.

The skilled person can easily adjust the culture medium to the nutritional requirements of the bacteria to be cultivated.

A broad range of solid or liquid culture media are known and may be used in the context of the present invention. Suitable media include liquid media and solid supports. Liquid media generally comprise water and may thus also be termed aqueous media. Solid media may comprise a polymeric support such as agar. Preferably, the culture medium is a liquid culture medium.

In a particular embodiment, the culture media comprises amicase, yeast extract, mineral solution, potassium phosphate, sodium chloride, ammonium sulfate, magnesium sulfate, calcium chloride nutriose, inulin, soluble potato starch, cellobiose, hemin, resazurin, vitamin, cystein HCl and/or NaHCO₃.

In particular embodiments, the producing method of the invention is performed in a reactor. By “reactor” is meant a conventional tank or any apparatus or system for fermentation and/or bioconversion, typically selected from bioreactors, biofilters, rotary biological contactors, and other gaseous and/or liquid phase bioreactors. The apparatus which can be used according to the invention can be used continuously or in batch loads, for example for batch or fed-batch fermentation. Batch cultivation such as in an anaerobic batch or fed-batch fermentation process is known to be particularly suitable for large-scale production of bacteria and bacteria consortia.

The invention thus also concerns a reactor or bioreactor comprising a consortium or composition as defined herein.

Preferably, the method of production is conducted under anaerobic conditions.

In some embodiments, the method according to the invention, the bacterial strains are cultured/multiplied by co-cultivation in an anaerobic batch fermentation process or in an anaerobic fed-batch fermentation process.

The term “batch fermentation” is known and denotes a fermentation process in a bioreactor, wherein during the fermentation process no material is removed from nor added to the bioreactor. In this text, the term “batch fermentation” in particular denotes a fermentation process, wherein there is no removal of a culture suspension cultivated in the bioreactor with the exception of insignificant amounts required for analytical testing, and wherein there is no addition of fresh culture medium into the bioreactor. Furthermore, a flow of gaseous compounds into and out of the bioreactor during the fermentation process, for example inflow of inert gas to maintain anaerobic cultivating conditions or such as outflow of metabolic exhaust gas, are not considered as material added or removed from the bioreactor.

The term “fed-batch fermentation” is known and denotes a fermentation process in a bioreactor, wherein during the fermentation process no material, in particular no-culture suspension is removed from the bioreactor, except for insignificant amounts required for analytical testing and except for gaseous compounds. However, in a fed-batch fermentation process, material is added to the bioreactor during the fermentation process, in particular fresh culture medium is added. The added culture medium may be the same or different culture medium as the culture medium in the bioreactor at the beginning of the fed-batch fermentation process.

In the method of the invention, the consortium may be amplified from an inoculum. Preferably, the bacterial strains are in the form of an inoculum from a prior continuous anaerobic co-cultivation process.

As used herein, the term “inoculum” refers to a sample containing viable bacteria, intended to be introduced into an environment favorable to its multiplication, preferably a suitable culture medium, in order to produce a greater quantity of said viable bacteria or to produce a compound produced by said bacteria, for example such as butyrate. The culture is preferably conducted in an industrial scale, i.e., in particular above 200 ml, above 300 ml or above 500 ml and more preferably in a volume of at least 1 L, at least 10 L, at least 30 L, at least 50 L, at least 100 L, at least 250 L or at least 500 L.

The terms “continuous culture”, “continuous cultivation” and “continuous co-cultivation” are known and refer to a cultivation of bacterial strains in a bioreactor comprising a liquid culture medium wherein during the cultivation process, materials are added and removed. In particular, the term “continuous culture” refers to a cultivation process wherein fresh medium replaces an equal volume of effluent of culture-suspension at a constant flow rate during the cultivation process.

The method according to the invention may further comprise the harvest or collection of the consortium, in particular after multiplication.

The method according to the invention may further comprise one or more post-treatment step. The term “post treatment” preferably refers to a further processing step or downstream treatment, such as for example a preservation treatment. The post-treatment can be cryopreservation or lyophilization.

In some embodiments, the method of the invention comprises a cryopreservation step and comprising:
mixing the harvested culture-suspension with a cryoprotective solution, in particular in order to obtain a 1:1 (v/v) mixture of culture-suspension and cryopreservant, preferably glycerol, or
centrifuging the harvested culture-suspension and resuspending an obtained pellet in a mixture of the cryoprotective solution and the dispersing medium, in particular in a 1:1 (v/v) mixture of cryopreservant, preferably a mixture of glycerol and dispersing medium, and
shock freezing with liquid N2 or gradually freeze to a storage temperature of at least −20° C., in particular at −20° C. to −80° C.

In another embodiment, the post-treatment is lyophilization comprising the steps of:
centrifuging the harvested culture-suspension and washing the obtained pellet with a buffer solution;
resuspending the pellet in a lyophilization solution and lyophilizing; and
subsequently, storing at a temperature of 4° C. or lower, or at room temperature.

In a very particular embodiment, the method of manufacturing the consortium or composition as defined herein, comprises the following steps:
I. providing a sample of the consortium of bacterial strains as an inoculum from a prior continuous anaerobic co-cultivation process;
II. adding the inoculum to a dispersing medium in a bioreactor thereby forming a culture-suspension of the bacterial strains;
III. multiplying the bacterial strains in the culture suspension by co-cultivation in an anaerobic batch fermentation process or in an anaerobic fed-batch fermentation process;
IV. optionally harvesting the consortium of bacterial strains; and
V. optionally, subjecting the harvested consortium to one or more post-treatment steps such as cryopreservation or lyophilization.

In some embodiments, such method further comprises a step of harvesting the consortium of bacterial strains.

In some embodiments, such method additionally comprises a step of subjecting the harvested consortium to one or more post-treatment steps such as cryopreservation or lyophilization, in particular as described hereabove.

### Kits

Compositions of the disclosure can be packaged as a kit. Then, the invention also provides a kit comprising the consortium of the invention in particular for the preparation of a composition such as disclosed herein.

In some embodiments, the kit may additionally comprise instructions for cultivation/multiplication of the consortium according to the invention, in particular when the consortium is provided in the kit as an inoculum.

The kit may additionally comprise instructions for preparing the composition such as disclosed herein.

The kit may alternatively comprise a composition comprising the consortium of the invention, in particular a pharmaceutical composition.

In some embodiments, the kit may include a composition as an already prepared dosage form ready for administration or, alternatively, can include a composition comprising the microbial consortium as described as a solid composition (e.g., in a lyophilized form) that can be reconstituted with a solvent to provide a liquid dosage form or as an inoculum for cultivation purposes.

In some embodiments, the kit is for use in treating, preventing, ameliorating, reducing or delaying the onset of an intestinal dysbiosis or of a disease or disorder related to an intestinal dysbiosis, such as a disease or disorder selected from the group consisting of Crohn's disease, inflammatory bowel disease, gastritis, colitis, ulcerative colitis, irritable bowel syndrome, cancer including gastro-intestinal cancer or colorectal cancer (CRC), ulcers such as gastric ulcer or duodenal ulcer, intestinal infections such as caused by viruses or bacteria, auto-immune disease, gastroenteritis, Guillain-Barre syndrome, graft versus host disease (GvHD), gingivitis, nosocomial infection, Clostridium difficile infection (CDI), infection by vancomycin resistant enterococci VRE) and post-infectious diarrhea; preferably selected from the group consisting of inflammatory bowel diseases (IBD), ulcerative colitis (UC) and Crohn's disease (CD).

In some embodiments, the kit of the invention may additionally comprise instructions for using the kit in treating an intestinal dysbiosis or of a disease or disorder related to an intestinal dysbiosis, such as described herein.

All the references cited in this description are incorporated by reference in the present application. Others features and advantages of the invention will become clearer in the following examples which are given for purposes of illustration and not by way of limitation.

### BRIEF DESCRIPTION OF THE FIGURES

The invention will be better understood when consideration is given to the figures and the following detailed description thereof.

FIG. 1: Simplification of the carbohydrate fermentation backbone of the human intestinal microbiome. Shown is a schematic illustration of the key functions of the intestinal microbiome and shows the pathways that are performed by bacteria of the intestinal microbiome. (A1) corresponds to the conversion of primary substrates and production of formate; (A2) corresponds to the conversion of primary substrates and production of acetate; (A3) corresponds to the conversion of primary substrates and production of butyrate; (A4) corresponds to the conversion of primary substrates and production of lactate; (A5) corresponds to the conversion of primary substrates and production of propionate; (A6) corresponds to the conversion of primary substrates and production of succinate; (B1) corresponds to the conversion of formate and production of acetate; (B2) corresponds to the conversion of acetate and production of butyrate; (B3) corresponds to the conversion of lactate and production of butyrate; (B4) corresponds to the conversion of lactate and production of propionate; (B5) corresponds to the conversion of succinate and production of propionate; (C1) corresponds to the conversion of primary substrates to lactate while reducing oxygen; (C2) corresponds to the conversion of H2, CO2 and formate to acetate. Modifications with regard to strains able to perform (B2), (B3) and (B4) metabolic functions can be targeted to favor conversion of intermediate metabolites, in particular lactate into butyrate.

FIG. 2a: Metabolite concentrations during continuous fermentations of the consortium C21 and two closely related consortia (C22, C23), each consortium in duplicates (mean values). All six fermentations contained only the end-metabolites acetate, propionate and butyrate and none of the intermediate metabolites succinate, lactate and formate after day 3. This is a strong indication that the trophic network for carbohydrate metabolism is functioning as hypothesized.

FIG. 2b: Stability of consortia C21 to C23 as measured by taking the coefficient of variation (CV) of 3 consecutive timepoints (sd/mean*100). A consortium is defined as stable (i.e., having reached equilibrium, or stable state) when the CV is below 10%. Based on this criterion, C21 was deemed stable starting at day 6 except for minor variations above 10% while C22 was deemed stable after 14 days. C23 took about 13 days to remain under 10% CV for all metabolites (each consortium in duplicates).

FIG. 3: Nanopore 16S sequencing allows for relative quantification of all strains present. All inoculated strains were present in every consortium until day 15. The numbers refer to B. xylanisolvens (1), Ruminococcus bromii (2), Agathobacter rectalis (3), Phascolarctobacterium faecium (4), Blautia hydrogenotrophica (5), Anaerostipes caccae (6), Lacticaseibacillus rhamnosus (7), and Bifidobacterium adolescentis (8).

FIG. 4: A consortium having selected butyrate producers able to perform (B2), (B3) functions such as Agathobacter rectalis and Anaerostipes caccae shifts intermediate metabolite consumption towards desirable end metabolites. Shown is the concentration of intermediate and end metabolites (SCFAs) in the effluent (y-axis, [mM]) in a continuous fermentation in a bioreactor over time (x-axis, [days]) by consortium C2 (Ruminococcus bromii, Faecalibacterium prausnitzii, Lactobacillus rhamnosus, Bifidobacterium adolescentis, Anaerotignum Eubacterium limosum, Collinsella aerofaciens, lactatifermentans, Phascolarctobacterium faecium, Blautia hydrogenotrophica) in comparison to C21 (having Agathobacter rectalis instead of F. prausnitzii and Angerostipes caccae instead of E. limosum). Consortium C21 showed significantly higher butyrate production than consortium C2 throughout the fermentation. At the same time, propionate production of C21 remained inferior to the one measured for C2.

FIG. 5: The effect of A. rectalis and A. caccae replacing F. prausnitzii and E. limosum exceeds the one induced by removal of (B4)-Function with regard to butyrate production. Shown is the concentration of intermediate and end metabolites in the effluent (y-axis, [mM]) of a continuous fermentation over time (x-axis, [days]) by consortium C2 in comparison to C15 (having A. rectalis instead of F. prausnitzii and A. caccae instead of E. limosum, but maintaining Anaerotignum lactatifermentans as (B4)-metabolizer). Consortium C15 showed higher butyrate production than C2 even though A. lactatifermentans as a representative of functional group B4 was maintained in the inoculum.

FIGS. 6a and 6b: Anaerobutyricum hallii shows a metabolic profile similar to the one of Anaerostipes caccae when grown for 48 hours on YCFA and YCFA supplemented with lactate. In particular, the metabolic profile of A. hallii and A. caccae differs significantly from the one of E. limosum when grown on LT-supplemented medium. While A. hallii and A. caccae produce almost exclusively butyrate, E. limosum is shown to be a major acetate producer.

FIGS. 7a and 7b: Agathobacter novel genus is a bacterial strain which could not be assigned to a genus described in the art. The strain is disclosed herein by means of the full genome sequence (i.e., SEQ ID No. 1), by 16SRNA sequence (i.e., SEQ ID NO:9), by gram stain, and by metabolic profile (total carbon converted into each of the major metabolites succinate, lactate, formate, acetate, propionate, butyrate and ethanol when grown for 48 hours on YCFA).

FIG. 8A,B: All C21 strains fully engraft in germ-free mice. Abundance of the consortium strains in the cecum content of germ-free mice 15 or 16 days after gavage reveals complete engraftment of C21 consortium. 8A:

Relative abundance of strains in cecum content measured by 16S sequencing (Nanopore, each line represents a mouse). 8B: Cell concentrations of Bifidobacterium adolescentis (SKRVN; measured by qPCR because the method used for Nanopore sequencing does not amplify Bifidobacterium sp.) and Ruminococcus bromii (SWYUN), P. faecium (SDVAP) and L. rhamnosus (SKFKT) for reference.

FIG. 9: The relative abundances of strains at equilibrium in the continuous co-cultivation production process (gavage sample) are strongly preserved when the fresh C21 product is administered to germ free mice (single application). The Figure shows composition in mouse feces measured at day 7 after administration of fresh C21. The fecal composition matches the reactor composition despite different conditions and substrates, and remains stable until day 10 (end of the study, not shown) indicating that the compositional equilibrium reached by the consortium during production (continuous fermentation) is representative of the equilibrium of the consortium in germ-free mice. The listed strains are SWYUN=R. bromii; SKFKT=L. rhamnosus; SKRVN=B. adolescentis; SDVAP=P. faecium, STEDK=A. caccae; SUMTT=B. xylanisolvens; SWUPP=A. rectalis, SIBFJ=B. hydrogenotrophica.

FIG. 10A, B: C21 induces reduction of cecum weight as opposed to placebo, recapitulating a part of the physiological effects of FMT. Cecal weight was reduced in germ free (GF) mice colonized with C21 after 14 days as opposed to placebo control. This suggests that administration of C21 contributed to partially restoring a normal intestinal morphology in colonized germ-free mice.

FIG. 11: Short chain fatty acids (SCFA) concentrations in cecal content of mice increase after gavage of C21 test product fresh from bioreactor (single application), compared to placebo. Circles show the posterior mean estimates for the concentrations of SCFA and the lines the 95% highest probability density intervals. NEG control: PBS (placebo) treated germ-free mice; POS control: SPF mice. The metabolic activity in the cecum of germ-free mice 14 days after administration of C21, especially the concentration of the SCFA acetate, butyrate and propionate was higher than in the placebo-treated mice, suggesting that C21 can initiate fermentation activity in the cecum, recapitulating the effect of FMT.

FIG. 12: C21 reduces the dominance of Enterococcus (GvHD risk model). There is an Enterococcus dominance in patients at risk of developing GvHD. Based on this model, germ-free mice were administered both Enterococcus and C21. Each circle, square, triangle, star shows the total bacterial abundance (closed symbols) or the absolute Enterococcus abundance (open symbols). Triangles and squares: mice were first colonized with enterococcus and subsequently treated with C21 (triangles) or FMT (squares); Circles and stars: vice versa, mice were first colonized with C21 (circles) or FMT (stars) and subsequently colonized with enterococcus. The goal of the study was to verify whether C21 can compete with Enterococcus in a comparable manner to FMT (that is curative in Enterococcus overgrowth associated with aGvHD). Full engraftment of consortium C21 in germ-free mice was observed in both cases, pre-colonization of mice with Enterococcus or challenge after colonization. C21 partially recapitulates the effect of FMT on overall bacterial abundance and reduced relative abundance of Enterococcus.

FIG. 13: A,B. B. hydrogenotrophica and E. callanderi display a similar metabolic profile when grown in single strain culture.

FIG. 14: Consortia with B. hydrogenotrophica or E. callanderi as a formate to acetate converter both show pronounced butyrate production in in vitro fermentation.

FIG. 15: Consortia with B. hydrogenotrophica, Blautia_A wexlerae (C36) and Terrisporobacter glycolicum (C34) as a formate to acetate converter show pronounced butyrate production in in vitro fermentation.

FIG. 16: Purposeful selection of the acetate to butyrate converters (B2 function) and lactate to butyrate converters (B3 function) can synergistically interact to maximize butyrate production. In particular, the total butyrate concentration after 48 hrs of a selected B2 converter (A. rectalis=SWUPP) in combination with a selected B3 converter (A. caccae=STEDK; A. hallii=SSJFA), total butyrate concentration exceeds the sum of butyrate accumulation in single strain cultures of A. rectalis, A. caccae, A. hallii under the same conditions.

### EXAMPLES

### Example 1: Rationale, Metabolic Pathways and Functional Groups

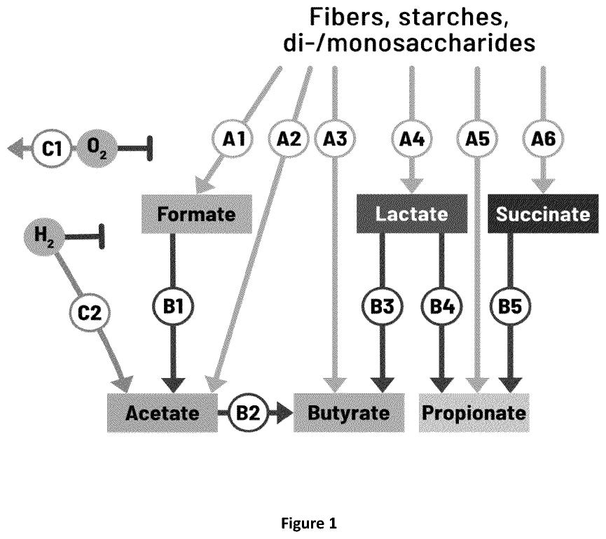

The combination of more bacteria strains performing the metabolic pathways as disclosed herein was chosen to (see FIG. 1):
degrade the main energy sources in the gut including fibers and intermediate metabolites (groups A1-A6);
protect anaerobiosis by reducing the eventual 02 through respiration (C1) and reduce H2 concentration (C2);
produce the main end metabolites found in the intestine (B1-B5);
prevent the enrichment of intermediate metabolites (formate, lactate, succinate) independently of the composition of the recipient's microbiome.

To assign the respective A or B reactions, consumption and/or production of fatty acids was measured using high-pressure liquid chromatography with refractive index (HPLC-RI). Literature data on hydrogen utilization or oxygen tolerability were used to assign C-reactions to the strains whereby, typically, oxygen reduction was assigned to strains reported to grow under aerobic or microaerophilic conditions and hydrogen consumption to acetogenic or methanogenic gut bacteria.

The inventors were able to define several different consortia that reflect the key functions of bacteria of an intestinal microbiome.

### Example 2: Identification of Bacterial Strains

Bacterial strains were isolated from healthy donors using anaerobic Hungate culturing techniques (Bryant, 1972) and characterized for growth and metabolite production on YCFA or M2GSC medium and modifications thereof: 1) Yeast extract, casitone, fatty acids medium (YCFA); and 2) glucose, soluble starch, cellobiose medium (M2GSC); 3-5) variants of M2GSC, where the ‘GSC’ carbon sources were exchanged by the intermediate metabolites lactate (M2LT), formate (M2FO), and succinate (M2SU), respectively. Cultivation was strictly anaerobic in Hungate tubes, at 37° C. and pH 6.5-7. Functions A1-A6 (i.e., pathways A1-A6) were assigned to the strains based on media 1 and 2, and the reactions B1-B5 based on growth in media 3-5. Growth was assessed after 48 h on media 1 and 2 and after 48 h and 7 days on media 3-5 to account for typically slow utilization rates for intermediate metabolites. The YCFA and M2GSC media were prepared as described in literature (Duncan et al., Roseburia intestinalis sp. Nov., a novel saccharolytic, butyrate-producing bacterium from human faeces, International Journal of Systematic and Evolutionary Microbiology 52, 1615-1620 (2002); Miyazaki et al., Degradation and utilization of xylans by the rumen anaerobe Prevotella bryantii (formerly P. ruminicola subsp. Brevis) B14. Anaerobe 3, 373-381 (1997)). M2SU, M2LT, and M2FO contained 30 mM of sodium succinate (SU), DL-lactate (LT), or formate (FO), respectively. Liquid media were boiled, flushed with 100% O2-free CO2, dispensed into CO2-flushed Hungate tubes, sealed and autoclaved before use.

The inventors were able to select particular bacterial strains performing the different metabolic pathways disclosed herein.

### Example 3: Analysis of Metabolic Activity Using HPLC_RI

The concentrations of SCFAs (propionate, acetate, butyrate), intermediate metabolites (formate, lactate, succinate) and ethanol were measured using HPLC-RI. Samples were centrifuged (14,000 g, 10 min, 4° C.) filtered into 1.5 mL short thread vials with crimp caps (VWR International GmbH, Schlieren, Switzerland) using 0.2 μm regenerated cellulose membrane filters (Phenomenex Inc., Aschaffenburg, Germany). Analyses were performed with a VWR Hitachi Chromaster 5450 RI-Detector using a Rezex ROA-Organic Acid (4%) precolumn connected to a Rezex ROA-Organic Acid (8%) column, equipped with a Security Guard Carbo-H cartridge (4×3.0 mm). Volumes of 40 μL were injected into the HPLC with a flow rate of 0.6 mL/min at a constant column temperature of 80° C. using a mixture of H2SO4 (10 mM) and Na-azide (0.05 g/L), as eluent. Concentrations were determined using external standards by comparing the retention time. Peaks were integrated using the EZChromElite software (Version V3.3.2.SP2, Hitachi High Tech Science Corporation). The limit of detection was defined as >0.8 mM for succinate, lactate, and acetate or >0.5 mM for all other SCFAs.

Characterization of metabolites production and/or degradation was assessed for each of the selected bacterial strains (data not shown).

### Example 4: Preparation of Single Strains for Inoculation

In order to establish the exemplary consortia, one strain from each functional group (i.e., performing the pathways described herein) was selected and combined for co-cultivation. For the inoculation of bioreactors, strain pre-cultures of the selected strains were grown. Therefore, 0.1 mL of cryopreserved isolates (1 ml of 48 h old culture mixed with 1 ml of respective fresh medium containing 60% of glycerol, stored at −80° C.) were inoculated in Hungate tubes containing 8 mL of the respective preferred growth medium of the strain. Optical density measurements confirmed growth.

Strain viability was assessed by flow cytometry and inoculation voluminal were chosen to achieve equal live cell numbers for each strain (data not shown). As an exception, in order to improve the competitiveness of slow-growers and prevent growth inhibition through inhibitory concentrations of intermediate metabolites, the slow-growing secondary degraders (B. hydrogenotrophica, A. caccae, P. faecium and A. rectalis) were added in a 10-fold volume.

### Example 4: Co-Culture in Fermentation Medium

The medium used for co-cultivation of the consortium was composed as presented in Table 1:

TABLE 1
Composition of PBMF010

| PBMF010 | 1 L | | Supplier |
|---|---|---|---|
| Amicase | 5 | g | Sigma-Aldrich |
| Yeast extract | 2.5 | g | Merck |
| Mineral solution I | 150 | mL | |
| Mineral solution II | 150 | mL | |
| Nutriose FB 06 | 2 | g | Roquette |
| Inulin (Fibrulose F97) | 1 | g | Coscura |
| Soluble potato starch | 1.5 | g | Sigma-Aldrich |
| Cellobiose | 1.5 | g | Sigma-Aldrich |
| Hemin stock solution (0.5 mg/mL) | 200 | μL | |
| Resazurin stock solution (1 mg/mL) | 1000 | μL | |
| Vitamin solution (1 mg/mL) | 1000 | μL | |
| Cystein HCl | 1 | g | Sigma-Aldrich |
| NaHCO3 | 4 | g | Sigma-Aldrich |
| ddH2O | Fill up to 1 | L | |

It does not include meat extract or rumen fluid. The carbon sources consist of cellobiose (1.5 g/L), Fibrulose F97 (1 g/L, Cosucra-Group Warcoing SA, Warcoing, Belgium), soluble potato starch (1.5 g/L) and Nutriose® (2 g/L, Roquette Frères, Lestrem, France). For Hungate tube and serum flask experiments, the medium was buffered 3× to prevent rapid acidification of the culture.

### Example 5: Bioreactor Cultivation

C21, C22, C23 consortia were each fermented in duplicates leading to a total of 6 fermentations. Controlled fermentations were carried out in 0.5 L Sartorius qPlus microbial version bioreactors (Sartorius AG, Göttingen, Germany, in duplicates). To maintain anaerobiosis, bioreactors were continuously flushed with CO₂. Medium feed, temperature, pH, stirring speed, and base consumption were monitored throughout the fermentation using the supplier's software EVE or DCU (Sartorius AG). pH was maintained by the automatic addition of 2.5 M NaOH.

The bioreactors were operated for a total of 17 days. The fermentations were started with a standard fed-batch process of an initial 24 h batch phase with 250 mL, followed by an addition of 250 mL of fermentation medium and another 24 h batch fermentation. The reactors were subsequently switched to continuous mode for the remainder of the fermentation.

Fed-batch fermentation bioreactors were inoculated at 0.66% v/v of the initial medium volume corresponding to 0.33% inoculation on the final volume of the fed-batch fermentation. Fermentation conditions were chosen to reflect conditions of the human colon and prevent a possible washout of slow growing strains, at 37° C., pH=6.0, stirring (120 rpm), and a mean retention time of 50 h (D=0.02 h-1). The bioreactors were initialized with fed-batch mode for the first 48 h. In fed-batch mode, the first half of the fermentation medium was added at t=0 and the second half was added at t=24 h of fermentation.

After 48 h, bioreactors were set to continuous mode. Bioreactor effluent samples were taken daily unless otherwise noted, and strain composition was measured using qPCR and/or Nanopore sequencing and metabolites by HPLC-RI.

All consortia were able to be produced in such conditions.

### Example 6: Metabolic Activity of the Consortia C21-C23

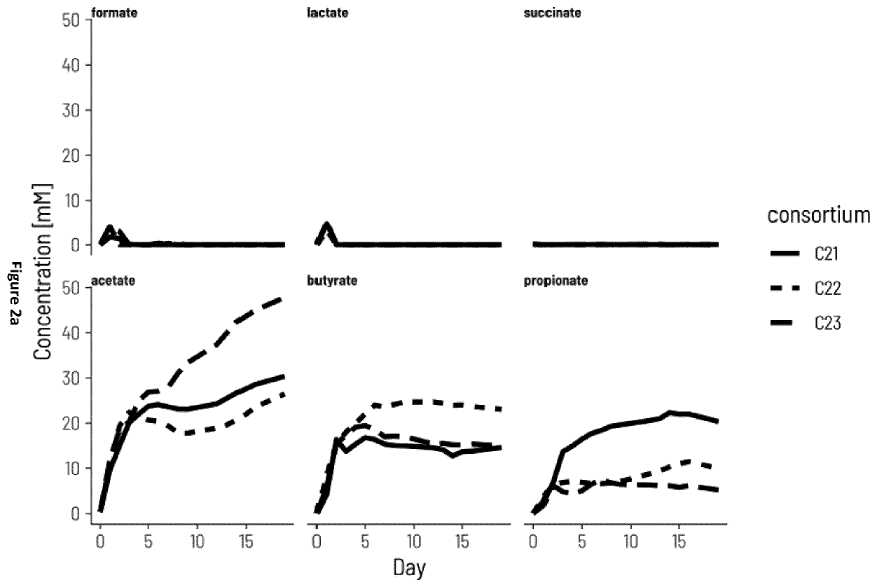

Growth was monitored by base consumption and optical density. Metabolites were monitored using HPLC-RI as described above. FIG. 2a shows metabolite concentrations during continuous fermentations of C21, C22 and C23, each in duplicates. The x-axis indicates cultivation time [days], the y-axis concentration in [mM]. All 6 fermentations contained only the end-metabolites acetate, propionate and butyrate and none of the intermediate metabolites succinate, lactate and formate after day 3. This indicates that the trophic network for carbohydrate metabolism is functioning as hypothesized. FIG. 2b shows stability of consortia C21 to C23 as measured by taking the coefficient of variation (CV) of 3 consecutive timepoints (sd/mean*100). A consortium is defined as stable (i.e. having reached equilibrium, or stable state) preferably when the CV is below 10%. Based on this criterion, C21 was deemed stable starting at day 6 except for minor variations above 10% while C22 was deemed stable after 14 days. C23 took about 13 days to remain under 10% CV for all metabolites.

C21-23 could therefore be cultivated in a bioreactor and showed the desired properties based on key metabolic pathways of the intestinal microbiome defined in FIG. 1, i.e. degradation of fibers and proteins into exclusively end-metabolites, a clear indication that the desired interactions and metabolic activities defined in (A1), (A2), (A4), (A6), (B1), (B2), (B3) and (B5) were established in a continuously operated bioreactor.

### Example 7: Strain Abundance Quantification

To assess the presence of all 8 bacterial strains of exemplary consortia C21, C22, C23 in the bioreactor over time, qPCR quantification of the single strains of each consortium was performed, using 16S metagenomic sequencing on Nanopore with the Nanopore 16S Barcoding Kit according to the manufacturer's protocol. Absolute abundances were calculated by performing qPCR on Lb. rhamnosus and subsequently multiplying the measured cell concentration with the relative ratios of all other strains. Because the primers in the 16S Barcoding Kit did not amplify Bifidobacterium sp.61, additionally B. adolescentis was quantified with qPCR directly.

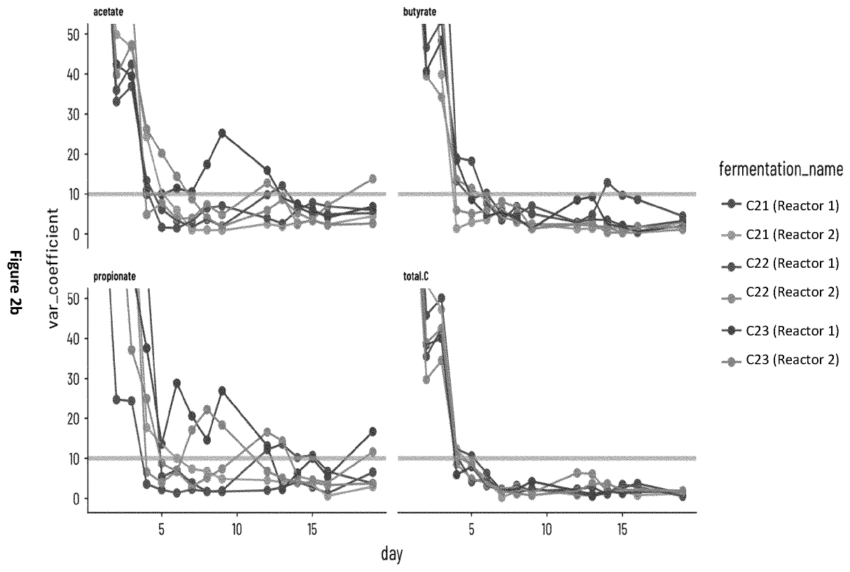

FIG. 3 shows the results of Nanopore 16S sequencing on day 15 of co-cultivation of the exemplary consortium C21. To assess the presence of all 8 bacterial strains in the bioreactor over time, relative abundance was determined by 16S metagenomic sequencing on Nanopore with the Nanopore 16S Barcoding Kit according to the manufacturer's protocol. The results (5 replicates) are shown. The numbers refer to B. xylanisolvens (1), Ruminococcus bromii (2), Agathobacter rectalis (3), Phascolarctobacterium faecium (4), Blautia hydrogenotrophica (5), Anaerostipes caccae (6), Lacticaseibacillus rhamnosus (7), and Bifidobacterium adolescentis (8).

All inoculated strains were present in every consortium until day 15, showing the stability of the cultivated strains. The same holds true for exemplary consortia C22 and C23 (data not shown).

### Example 8: Production of In Vitro Assembled Consortia Using Batch Fermentation

In addition to the continuous fermentations, all consortia (C21-C23) were cultured in batch fermentations in serum flasks. These fermentations were inoculated with the same strain mixes as for the continuous fermentations described above in order to assess the degree of predictability of the continuous fermentations from batch fermentations in serum flasks. The freshly prepared strain mix of each consortium was inoculated in 9 ml 3× buffered PBMF010 medium into serum flasks in triplicates. Medium was produced under gas atmosphere and reduced with cystein, headspace was filled with 100% CO₂ at ATM pressure. The serum flasks were incubated at 37° C. for 2 days and shaken. At 24 hours and 48 hours, the OD and pH were measured and samples were taken for metabolite analysis by HPLC.

The metabolic composition over time showed a slightly higher propionate production of C21 as compared to C22-23 (data not shown).

### Example 9: Viability of Consortium

To quantify the total amount of viable cells in the bioreactor, effluent was analyzed using a double-staining assay with the two nucleic acid dyes SYBR Green (SG) and propidium iodide (PI). Absolute counts were determined with a Millipore Guava Easycyte HT Flow Cytometer.

The inventors show that the consortium of cultivated bacterial strain has a good viability.

### Comparative Example 10: Butyrate Production of a Consortium According to the Invention is More Pronounced than in Consortia Known in the Art

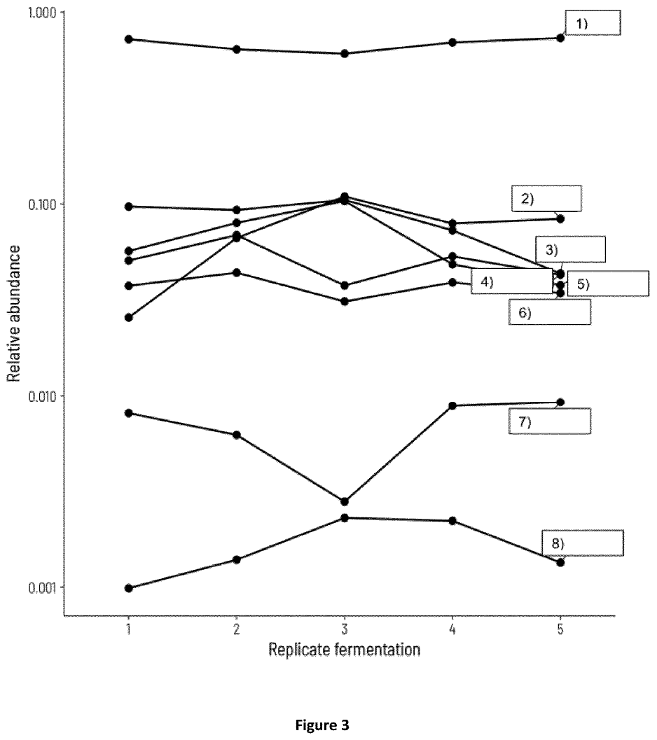

Composition comprising specific consortia of living bacteria strains selected such that a carbohydrate degradation metabolic network of the human intestinal microbiome is established which is stable over time and avoids accumulation of intermediate metabolites while reliably producing end metabolites has been described in the art (see WO2018/189284 and WO2020/079026). A consortium according to the invention displays enhanced butyrate production. This is shown in FIG. 4, where the known consortium C2 (in the cited publications referred to as “PB002”) was cultivated continuously and its metabolic profile was compared to the one of C21.

The bacterial strains used in each composition of example 10 and 11 are displayed in the following Table 2:

TABLE 2
Composition of the consortia and related metabolic pathways

| | Consortia | | | | |
|---|---|---|---|---|---|
| Pathways | C2 | C15 | C21 | C22 | C23 |
| A1, A2 | R. bromii | R. bromii | R. bromii | R. bromii | R. bromii |
| A1, A2, A4 | B. adolescentis/ | B. adolescentis/ | B. adolescentis | B. adolescentis | B. adolescentis |
| | C. aerofaciens | C. aerofaciens | | | |
| A4, C1 | Lb. rhamnosus | Lb. rhamnosus | Lb. rhamnosus | Lb. rhamnosus | Lb. rhamnosus |
| A6 | n/a | n/a | B. | P. copri | nov. genus |
| | | | xylanisolvens | | |
| B1, C2 | B. hydrogeno- | B. luti | B. hydrogeno- | B. hydrogeno- | B. hydrogeno- |
| | trophica | | trophica | trophica | trophica |
| B2 | F. prausnitzii | A. rectalis | A. rectalis | A. rectalis | A. rectalis |
| B3 | E. limosum | A. caccae | A. caccae | A. caccae | A. caccae |
| B4 | A. | A. | n/a | n/a | n/a |
| | lactatifermentans | lactatifermentans | | | |
| B5 | P. faecium | P. faecium | P. faecium | P. faecium | P. faecium |

C2 and C21 were composed and co-cultured as described above (Examples 3 to 5). In contrast to C21,22,23 cultures, C2 was fermented on a medium, composed as described above (table 1) but having resistant pea starch N-735 (2 g/L; Roquette Frères, Lestrem, France) instead of Nutriose. The metabolite activity was measured as described in Example 6. FIG. 4 shows that a consortium having selected butyrate producers able to perform (B2), (B3) functions according to the invention shifts intermediate metabolite consumption towards desirable end metabolites, i.e. butyrate. Shown is the concentration of intermediate and end metabolites (SCFAs) in the effluent (y-axis, [mM]) in a continuous fermentation in a bioreactor over time (x-axis, [days]) by consortium C2 in comparison to C21, having Agathobacter rectalis instead of Faecalibacterium prausnitzii and Anaerostipes caccae instead of Eubacterium limosum. Consortium C21 showed significantly higher butyrate production than Consortium C2 throughout the fermentation. At the same time, propionate production of C21 remained inferior to the one measured for C2.

Without wishing to be bound to theory, the inventors believe that the enhanced butyrate production is due to A. rectalis (B2) and A. caccae (B3) being particularly strong butyrate producers when co-cultivated in the context of the inventive consortium. The effect may further be amplified by the absence of any bacterial strain performing pathway (B4) (lactate to propionate) in C21. Lactate to propionate conversion function is assumed by a strain of Anderotignum lactatifermentans in comparative consortium C2.

### Comparative Example 11: The Enhanced Butyrate Production is Due to the Selection of Representatives of the (B2) and (B3) Metabolic Function

In order to get a better understanding of the reasons for the butyrate shift, butyrate production in C2 and C15 was compared. C15 is a modified version of C2 as displayed in the following table, with A. rectalis instead of F. prausnitzii and A. caccae instead of E. limosum, but maintaining A. lactatifermentans as (B4)-metabolizer) and C. aerofaciens of pathways (A1), (A2), (A4).

The continuous fermentation of C2 and C15, was conducted in a volume of 500 mL, each time. Seven mL of inoculum prepared as described above were added to 500 mL fresh medium. Two reactors were inoculated with C15 and one reactor was inoculated with C2, respectively. Incubation was performed in batch fermentation for 24+24 hours, as described above, followed by continuous fermentation (flow rate of 50 mL/h, constant volume of 500 mL). The fermentation of the two C15 replicates and C2 was conducted for 14 days. Every day, metabolite concentration was measured. OD, pH and RedOx were monitored daily. In addition, nanopore whole genome sequencing was carried out with C2 and C15 to analyze the presence and abundance of each strain at the end of the fermentation (14 days). After the fermentation was switched to continuous mode (Day 3), no accumulation of the intermediate formate, lactate and succinate metabolites was measured in any of the consortia C15 and C2.

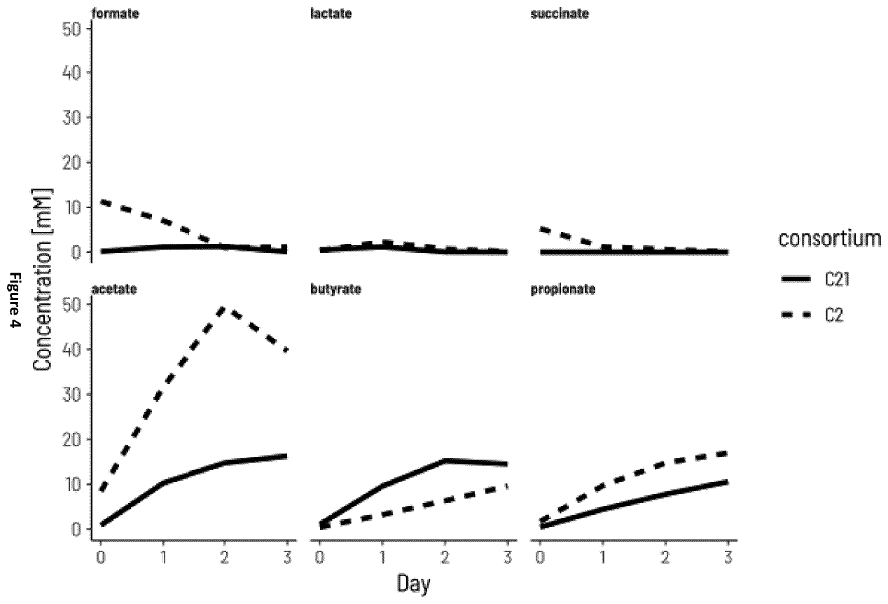

FIG. 5 shows the concentration of intermediate and end metabolites in the effluent (y-axis, [mM]) over time (x-axis, [days]) by consortium C2 in comparison to C15. Consortium C15 showed higher butyrate production than C2 even though A. lactatifermentans as a representative of functional group B4 (conversion of lactate to propionate) was maintained in the inoculum. The effect of A. rectalis and A. caccae replacing F. prausnitzii and E. limosum exceeds the one induced by removal of (B4)-Function with regard to butyrate production.

Hence the selection of strains assuming (B2) and (B3) metabolic functions appears to be decisive for the butyrate production.

### Example 12: Interchangeability of Anaerostipes Caccae and Anaerobutyricum hallii (B2)

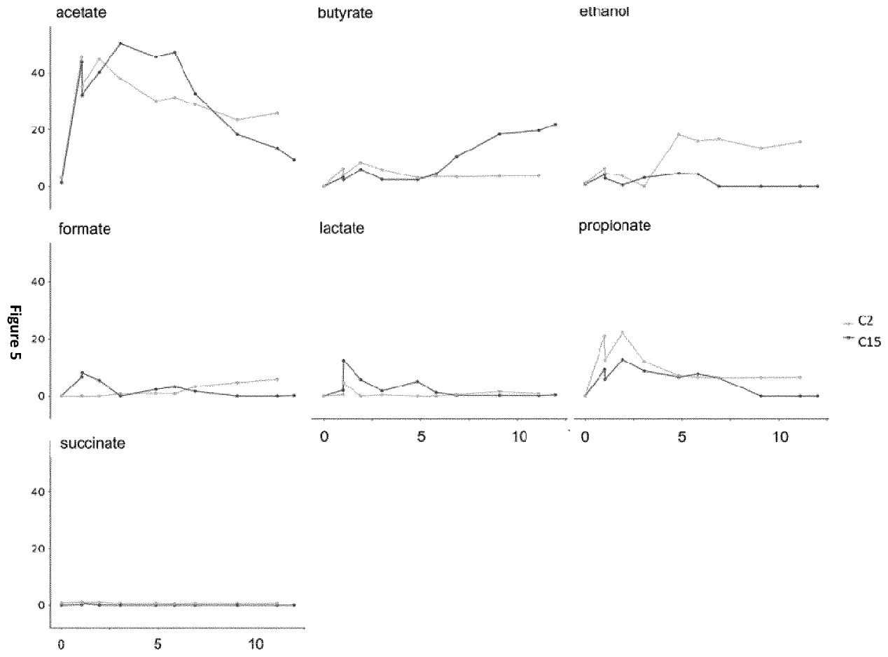

When assayed in single strain culture as described above, Anaerostipes caccae and Anaerobutyricum hallii display a similar metabolic profile. When grown in single strain culture on YCFA medium, it is characterized by pronounced acetate degradation and butyrate production (FIG. 6a). When grown on lactate supplemented media, it is characterized by moderate lactate and acetate conversion and strong butyrate production (FIG. 6b). In contrast, E. limosum which was promoted as a lactate to propionate converter in the past (PB002 consortium in WO2018/189284 and WO2020/079026), is in fact characterized by a major acetate production on both media.

Hence, A. hallii is a valid alternative to A. caccae for assuming the B2-function, conversion of lactate into butyrate and, in any case, exceeds the metabolic activity of previously suggested E. limosum in this respect.

### Example 13: Novel Genus of Acutalibacter

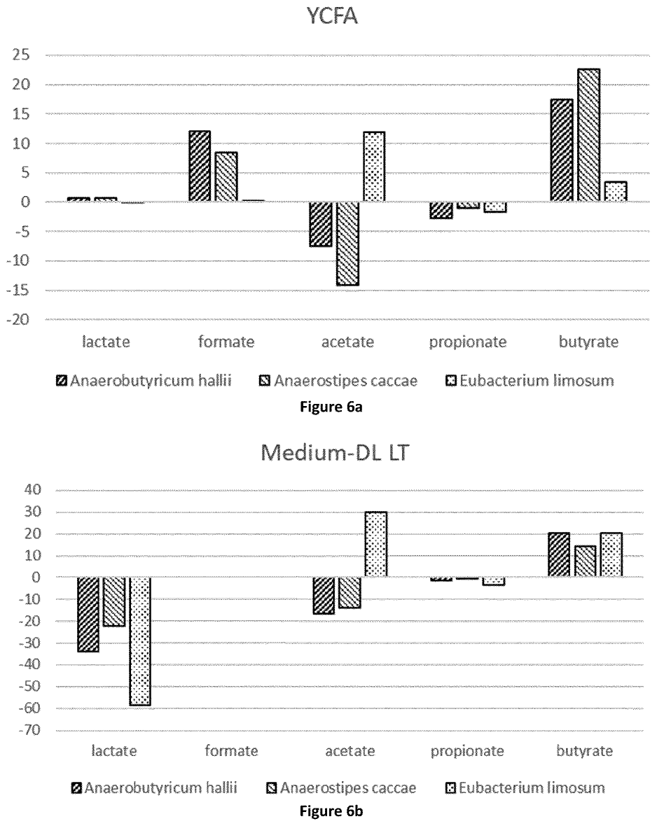

The bacterial strain(s) being able to convert primary substrates into succinate and which is included in consortium C23 as described herein is of a novel genus of Acutalibacter, which could not be assigned to a strain deposited in a reference database (such as ATCC, DMZ). The strain is disclosed herein by means of full genome sequence (as set forth in SEQ ID NO: 1) or 16SRNA sequence (as set forth in SEQ ID NO:9), by Gram stain (FIG. 7a) and by metabolic profile (percentage of total carbon converted into the metabolite of interest after at least 24 hrs growth in single strain culture on YCFA, see FIG. 7b).

### Example 14: Engraftment of C21 Consortium in Germ Free Mice

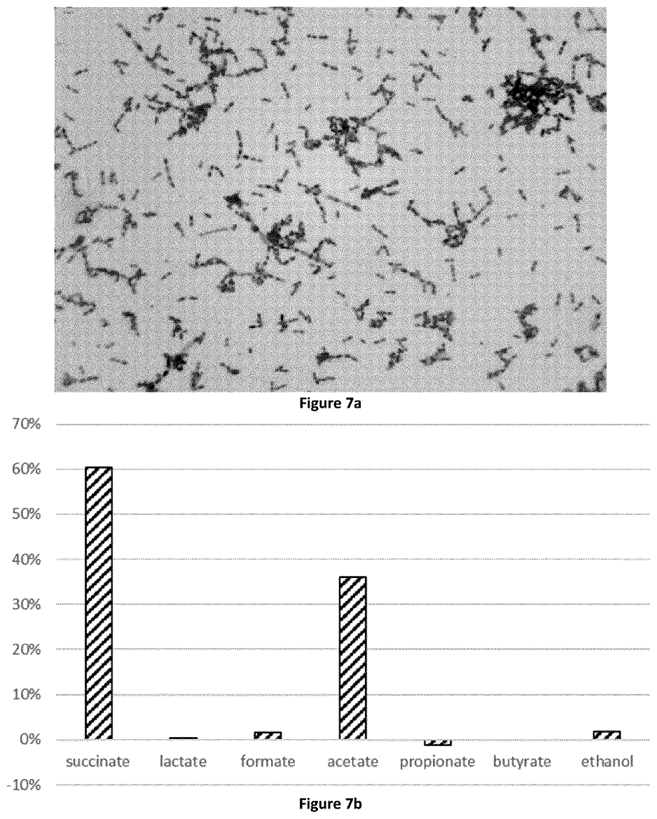

Engraftment of C21 (B. adolescentis, R. bromine, B. xylanisolvens, L. Rhamnosus, B. hydrogenotrophica, A. caccae, P. faecium, A. rectalis, co-cultured at 37° C., pH 6.0, fresh from Bioreactor) in wild-type C3H/HeN germ-free male mice was tested. 200 μL of C21V1 at 5.55·109 live cells/mL, representing 1.11·108 cells per administration was administered orally (gavage, single administration) to 8 mice of the test group. The complete consortium C21 (all strains) engrafted in 8 mice (presence in cecum at euthanasia, day 15 or 16). The presence of all strains was verified with either 16S amplicon sequencing (Nanopore) or qPCR for B. adolescents (SKRVN) and Ruminococcus bromii (SWYUN), P. faecium (SDVAP) and L. rhamnosus (SKFKT) for reference (FIG. 8A,B). All C21V1 strains were detected at the time of euthanasia in cecal samples of C21-treated mice. This suggests that all strains that are comprised in C21 engrafted in the mouse gastrointestinal (GI) tract. The strain abundances in the feces on day 6 and 14 were comparable (data not shown).

### Example 15: Comparison of Strain Abundances in the Fermentation from which the Test Item was Derived Versus in Fecal Samples from C21

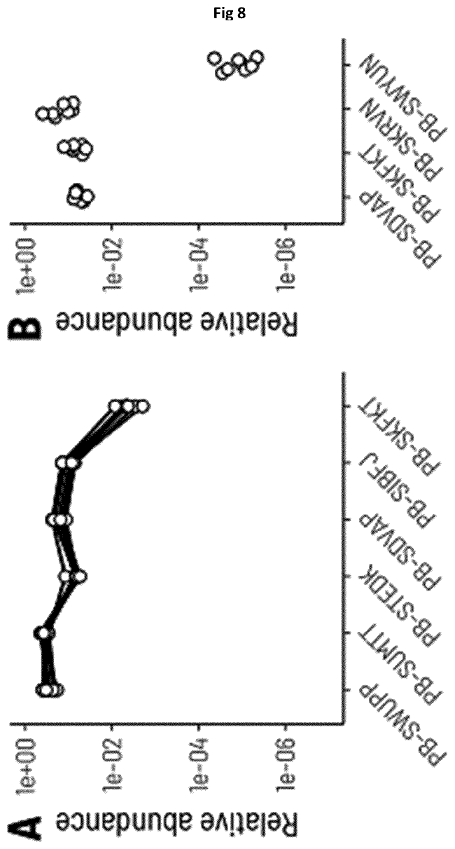

The equilibrium strain composition in mice (see example 14) was highly similar to the strain community composition in the bioreactor used for test item production. Bacteroides xylanisolvens (SUMTT) was the most abundant species in both mouse and fermentation samples with medians of respectively 67.3% and 62.2%. The following 4 strains had medians at similar orders of magnitudes in both sample types: Agathobacter rectalis (SWUPP, mouse: 10.5%; fermentation: 8.0%), Phascolarctobacterium faecium (SDVAP, mouse: 7.2%; fermentation: 2.7%), Blautia hydrogenotrophica (SIBFJ, mouse: 5.2%; fermentation: 6.8%), and Anaerostipes caccae (STEDK, mouse: 2.4%; fermentation: 3.6%). Lacticaseibacillus rhamnosus (SKFKT) was less abundant in both mouse and fermentation samples with medians of 0.4% and 0.8%, respectively. Bifidobacterium adolescentis had a higher relative abundance In mice than in fermentation samples (SKRVN, 6.4% and 0.26%, respectively). Ruminococcus bromii was below detection limit in the fecal samples (SWYUN, FIG. 9). The lines represent the minimum and maximum relative abundances in mouse fecal samples and in continuous fermentation from which the test item was derived. The bioreactor was sampled at the administration day, and on day 4 and 13 after administration, respectively. The points represent the median.

### Example 16: Comparative Treatment Studies/Cecum Weight

Comparative Treatment studies were performed as follows: Experimental group 1: C21 administered to 8 males (C21 gavage as described in Example 14); Control group 1: Placebo (autoclaved water, gavage on day 1), 8 males; Control group 2: Fecal microbiota transfer (FMT on day 1 by gavage) from specific pathogen free (SPF) mice, 8 males; Control group 3: SPF mice used for FMT preparation, 2 males. Half the mice were euthanized on day 15 and the other half on day 16 after gavage.

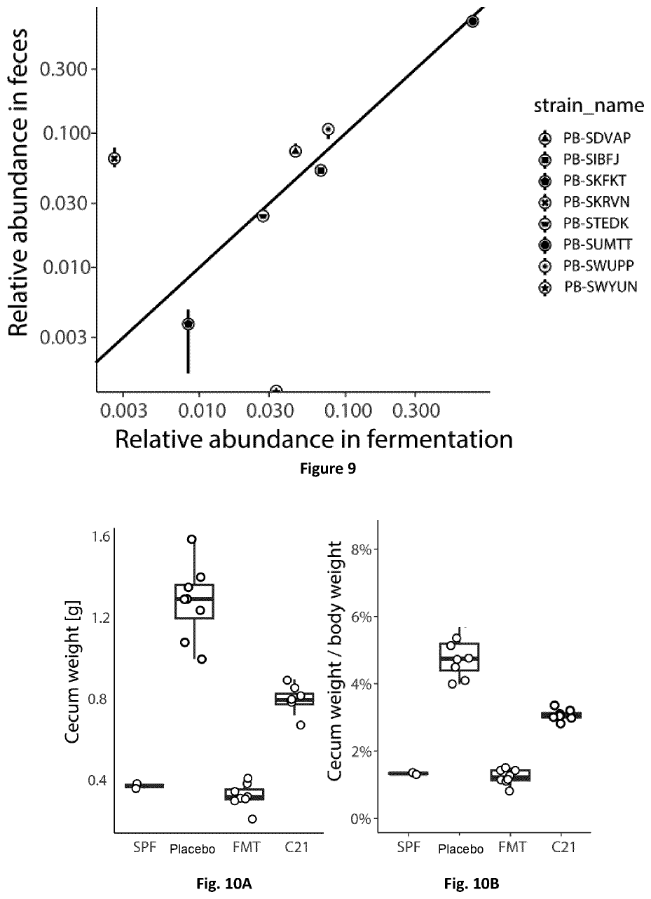

Cecum weights were assessed upon euthanasia and differed between treatments. C21-treated mice had a lower cecum weight than placebo-treated mice, but a higher cecum weight than mice that received FMT (FIG. 10A). There were no significant differences in cecum weight between FMT-treated mice and SPF mice. As body weights at euthanasia were indistinguishable between the groups, normalization of cecum weight to body weight led to equivalent results (FIG. 10B). For each of these parameters, the variation within groups was very small. It is well established in the literature that germ-free mice display a higher cecum weight than their SPF counterpart, in line with a reduction of cecum weight upon bacterial colonization (e.g. Darnaud et al., Nature Communications, 2021; El Aidy et al., Mucosal Immunology, 2012). Data from this current study suggest that germ-free mice administered an FMT from SPF mice had a similar cecum weight to that of SPF mice. 15 days after administration of C21 in germ-free mice, the cecum weight was significantly reduced compared to the placebo group, but remained higher than the one of SPF mice. This suggests that administration of C21 contributed to partially restoring a normal intestinal morphology in colonized germ-free mice.

### Example 17: Comparative Treatment Studies/SCFA Concentrations

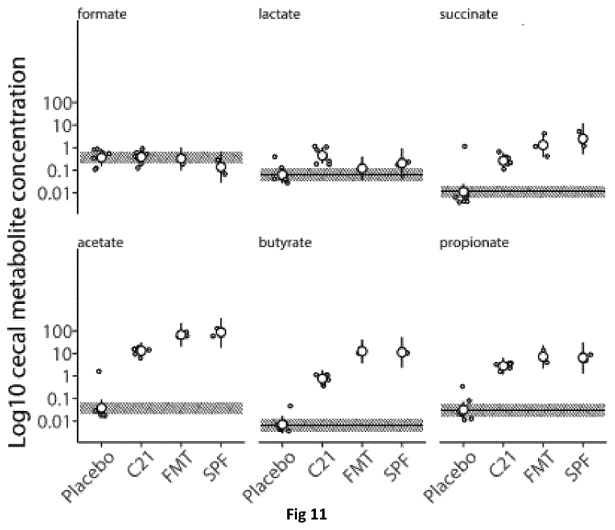

Short chain fatty acids (SCFA) concentrations in cecal content of the mice treated according to Examples 14 and 16 (purified and derivatized samples) was determined by LC-MS (concentration in [umol/g]; FIG. 11) and confirmed full engraftment and an SCFA production similar to FMT. Specifically, the metabolites formate (FO), lactate (LT), succinate (SU), acetate (AC), butyrate (BU), and propionate (PR) were measured. Placebo-treated mice (negative control) had cecal SCFA (AC, BU, PR) concentrations that were 3-4 orders of magnitude lower than SPF mice (positive control group). In C21 and FMT treated mice, acetate, butyrate, and propionate concentrations were significantly higher than in placebo treated mice. The concentrations in the FMT-treated mice did not differ significantly from the SPF mice. Concentrations in C21 mice were typically lower than FMT and SPF. In addition, C21 treated mice had concentrations of lactate that were significantly higher than the placebo group and FMT group. Overall, these data suggest that both C21 and FMT initiate fermentation activity in the cecum.

### Example 18: Concentration of Lipocalin 2 (Lcn2) in Feces

Lipocalin 2 (Lcn2) was quantified in the C21 and FMT treated groups, as well as the SPF mice of Examples 14 and 16 on day-1 (to define baseline levels), 7 and 14, using ELISA (“R&D mouse Lcn2 ELISA duo-set”, R&D Systems, Reference DY1857). Fecal Lcn2 concentrations did not change between day 3 and day 7 in the FMT-treated mice. This suggests that the onset of higher Lcn2 production in FMT-treated mice (indicative of innate immune response) occurred prior to the sample collection on day 3. We did not detect a significant difference in the Lcn2 concentration in feces to that in the cecum, suggesting that the concentration measured in feces is representative of that in the cecum. Cecal Lcn2 concentrations ([ng/g], data not shown) in the FMT-treated group were, however, significantly higher than those in the two SPF mice (negative control). This suggests that the increased Lcn2 concentrations in the FMT-treated mice is not merely a normalization of the Lcn2 production to “normal” higher levels, but indeed an active response. Lcn2 levels in feces were elevated in FMT-treated mice at days 7 and 14 compared to C21-treated and placebo-treated mice, and compared to Lcn2 levels at the start of the experiment. C21-treated and placebo-treated mice did not have elevated Lcn2 levels compared to the start of the experiment.

### Example 19: Competition Between C21 and Enterococcus faecium for Colonizing the Gut of Germ-Free BALB/c Mice

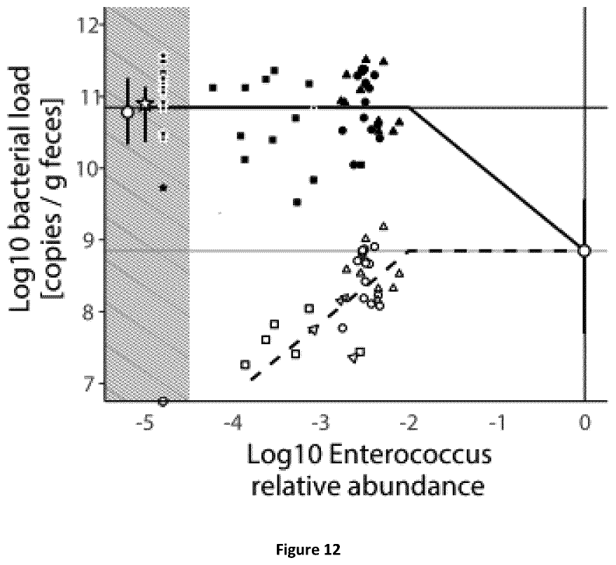

In patients undergoing hematopoietic stem cell transplantation (HSCT), Enterococcus sp. Are often present at subdominant levels prior to conditioning and can bloom after HSCT, likely contributing to acute graft versus host disease. Enterococcus blooms are driven by competition for substrate and growth inhibition of other microbiome strains from excessive lactate/formate production. We assessed whether the relative abundance of Enterococcus faecium (TX9) in gnotobiotic BALB/c mice mono-colonized with E. faecium (TX9) would decrease upon the preemptive or curative administration of C21. C21 and TX9 were administered to a mix of male and female mice (3-7 mice per group, single administration) with one week interval between each gavage. C21: 200 μL volume were administered either first on day 1 (5.38·109 live cells/mL, 1.08·108 cells per administration) or after enterococcus colonisation on day 8 (4.19·109 live cells/mL, 8.38·108 cells per administration). TX9: Single administration, 200 μL volume, either at day 1 or at day 8; ca. 108 cells per administration. All C21 strains engrafted in the cecal content of the mice (not shown). The administration of C21 reduces the dominance of Enterococcus in a gnotobiotic mouse model (FIG. 11). The solid symbols show the total bacterial load and the open symbols show the estimated Enterococcus load (stars and squares: FMT, circles and triangles: C21; The squares and triangles represent the experiment where mice were colonized with TX9 first, followed by FMT/C21 treatment; the stars and circles, conversely, represent preemptive treatment with TX9 second). The open point at x=0 (100% Enterococcus abundance) shows the bacterial load in mice mono-colonized with Enterococcus, and the open points at x=−5 show the bacterial loads in C21 (circle) and FMT (star) treated mice prior to Enterococcus administration. The solid black line shows the expected total bacterial load increase that is expected if Enterococcus absolute abundance is maintained up to a maximum bacterial load, and the dashed line shows the corresponding Enterococcus relative abundance (FIG. 12).

### Example 20: B. hydrogenotrophica can be Replaced by E. Callanderi for an Even More Pronounced Butyrate Production

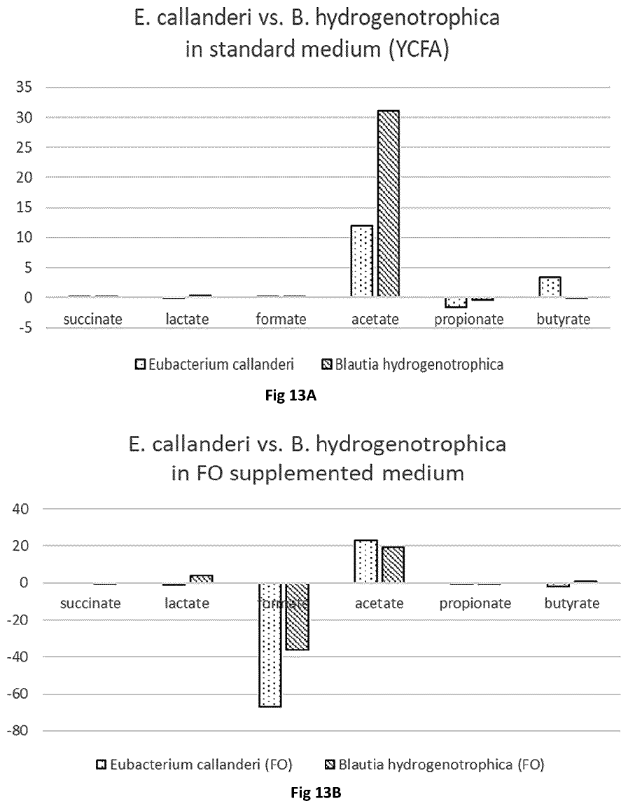

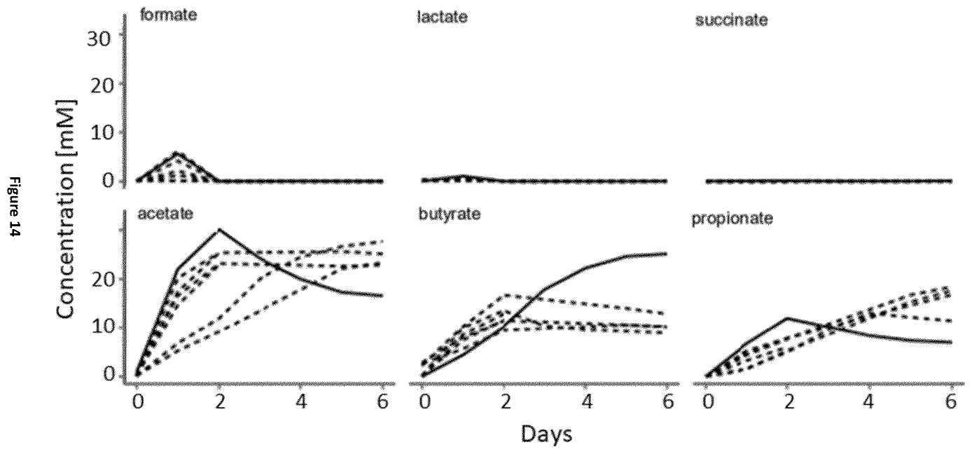

Inventors found that an equally good or even better representative of metabolic group B1 (formate to acetate, under consumption of H2) in comparison to B. hydrogenotrophica is E. callanderi. When grown in single strain culture as described above, E. callanderi and B. hydrogenotrophica display a similar metabolic profile. On YCFA medium, the profile is characterized by pronounced acetate production, accompanied by some butyrate production by E. callanderi (FIG. 13A). When grown on formate supplemented medium, it is characterized by strong formate conversion and acetate production (FIG. 13B). A similar result is obtained when consortia having the respective B1-representative are co-cultivated (FIG. 14). Specifically, the following consortia were compared:

| Function | C21 representative | C14 representative |
|---|---|---|
| A1 (primary to FO) | R. bromii | R. bromii |
| A2 (primary to AA) | B. adolescentis | B. adolescentis |
| A4( primary to LA) | L. rhamnosus | L. rhamnosus + Collinsella |
| | | aerofaciens |
| A5 (primary to PA) | n/a | Anaerotignum |
| | | lactatifermentans |
| A6 (primary to SU) | B. xylanisolvens | n/a |
| B1 (FO to AA) | B. hydrogenotrophica | E. limosum |
| B2 (AA to BA) | A. rectalis | A. rectalis |
| B3 (LA to BA) | A. caccae | A. caccae |
| B5 (SU to PA) | P. faecium | P. faecium |

The dashed lines represent C21 consortium fermentations, the solid line C14 shows C14 consortium fermentation. E. limosum ensures robust formate degradation, acetate production and even a more pronounced butyrate level after three days of in vitro fermentation compared to C21. Without wishing to be bound by theory, inventors hypothesize that butyrate production is higher and propionate production diminished in C14 (a) due to the lack of the primary substrate>SU (SU>PA) pathway, but also (b) due to the ability of E. limosum to convert propionate and produce butyrate under standard conditions (FIG. 13A). Hence, E. callanderi is a valid alternative to B. hydrogenotrophica for assuming the B1-function, and might in some constellations even be the preferred choice.

More alternative B1 performers (formate to acetate) could be identified in single strain experiments and their performance verified via readouts of fermentations of full consortia. Specifically, strains of the species Intestinibacter bartlettii, Terrisporobacter othiniensis and Blautia_A wexlerae could have been successfully identified as consuming substantially all formate present within 48 hours in single strains cultures when grown on FO-supplemented standard medium (M2,M2GSC) under standard conditions (data not shown). The result could have been confirmed in co-cultivation experiments of full consortia having a respective B1-representative. Specifically, the following consortia were compared:

| Function | C21 | C34 | C36 |
|---|---|---|---|
| A1 (primary to FO) | R. bromii | R. bromii | R. bromii |
| A2 (primary to AA) | B. adolescentis | B. adolescentis | B. adolescentis |
| A4( primary to LA) | L. rhamnosus | L. rhamnosus | L. rhamnosus |
| A6 (primary to SU) | B. xylanisolvens | B. xylanisolvens | B. xylanisolvens |
| B1 (FO to AA) | B. hydrogenotrophica | Terrisporobacter | Blautia_A |
| | | othiniensis | wexlerae |
| B2 (AA to BA) | A. rectalis | A. rectalis | A. rectalis |
| B3 (LA to BA) | A. caccae | A. caccae | A. caccae |
| B5 (SU to PA) | P. faecium | P. faecium | P. faecium |

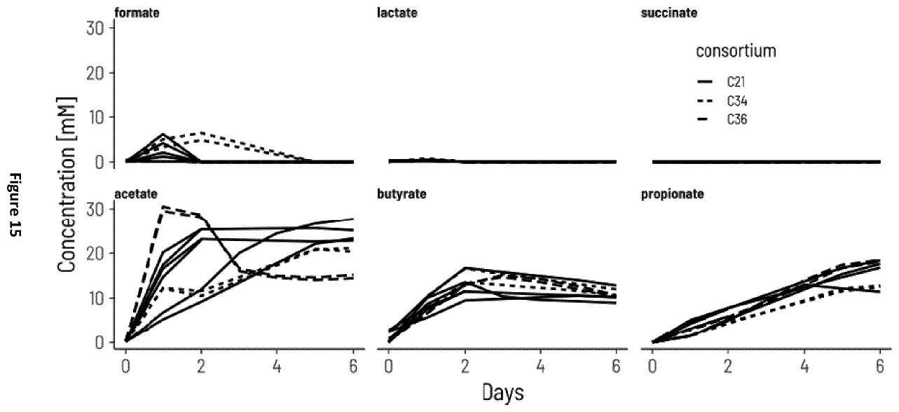

The consortia were cultivated in standard medium in serum flasks and bioreactors and sampled over 6 days in intervals of 24 hours. Metabolite concentrations were determined by HPLC-RI. The solid lines represent metabolite concentration in C21 fermentations, the dotted line in C34 fermentation, the dashed line in C36 fermentation (FIG. 15). Terrisporobacter othiniensis (C34) and Blautia_A wexlerae (C36) in their respective consortia ensure robust formate degradation, acetate production and similarly pronounced butyrate levels after six days of in vitro fermentation compared to C21. Blautia_A wexlerae (C36) strain is further disclosed by means of 16S RNA sequence (SEQ ID NO: 17).

### Example 21: Agathobacter rectalis and Anaerostipes caccae when Co-Cultivated Together Produce More Butyrate than Each of them Grown in Single Strain Culture

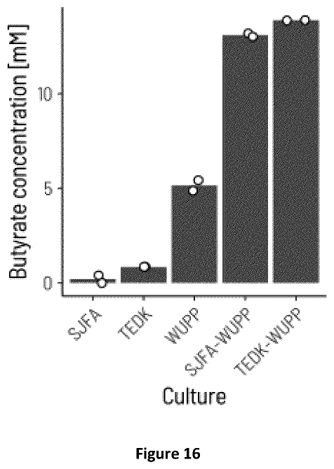

Agathobacter rectalis (SWUPP) and Anaerostipes caccae (STEDK) were reactivated and inoculated in 8 mL bYCFA_SS medium (YCFA medium with soluble starch as a carbon source, 3 g/L) to test for their butyrate producing ability as mono- and co-cultures. Fresh 24 h-old cultures of each strain were inoculated in duplicates at a ratio of 1:100 (v/v) in bYCFA_SS in Hungate tubes. In addition, co-cultures were premixed 1:1 (v/v) (PB-SWUPP+PB-STEDK) in two separate anaerobic Hungate tubes and were then inoculated in the same way as the monocultures. The Hungate tubes with the inoculated cultures were incubated at 37° C. for 48 h. After 48 h the cultures were analyzed by measuring the OD600 nm and the metabolic profile of the supernatant with HPLC-RI). The results HPLC-RI measurement are displayed in FIG. 16. The combination of A. rectalis (SWUPP) and A. caccae (STEDK) according to the present invention is more than an accumulation of two butyrate producers. A. rectalis and A. caccae synergistically interact to drive conversion of available substrate towards butyrate. The same applies to Anaerobutyricum hallii PB-(SSJFA) and A. rectalis (PB-SWUPP) in single culture vs co-culture. The metabolic profile of the co-culture goes beyond the sum of butyrate production by each strain taken in isolation.

1. A composition comprising a bacteria consortium of anaerobic bacterial strains, wherein said consortium comprises i) Agathobacter rectalis and ii) Anaerostipes caccae and/or Anaerobutyricum hallii and comprises no more than 15 different bacterial strains, for use in the treatment of an intestinal dysbiosis or of a disease or disorder caused by or related to an intestinal dysbiosis.

2. The composition for use according to claim 1, wherein the intestinal dysbiosis is selected from the group consisting of an intestinal dysbiosis following antibiotics treatment, an intestinal dysbiosis following infection by vancomycin resistant enterococci, an intestinal dysbiosis following infection by carbapenem resistant enterococci, and an intestinal dysbiosis following post-infectious diarrhea.

3. The composition for use according to claim 1, wherein the disease or disorder caused by or related to an intestinal dysbiosis is selected from the group consisting of inflammatory bowel disease, including ulcerative colitis and Crohn's disease; rheumatoid arthritis; multiple sclerosis; graft versus host disease; solid and liquid cancer, in particular gastrointestinal cancer, colorectal cancer and acute myeloid leukemia.

4. The composition for use according to any one of claims 1-3, wherein the consortium comprises i) Agathobacter rectalis and ii) Anaerostipes caccae and/or Anaerobutyricum hallii and iii) one or several bacterial strain(s) able to convert primary substrates into formate, lactate, succinate, acetate, butyrate and/or propionate, wherein the primary substrates are selected from the group consisting of sugars, starches, fibers and proteins and any combination thereof.

5. The composition for use according to claim 4, wherein the consortium comprises one or several bacterial strain(s) able to convert primary substrates into lactate.

6. The composition for use according to claim 4 or 5, wherein the consortium comprises:
one or several bacterial strain(s) able to convert primary substrates into formate;
one or several bacterial strain(s) able to convert formate into acetate; and/or
one or several bacterial strain(s) able to convert primary substrates into acetate.

7. The composition for use according to any one of claims 4-6, wherein the consortium comprises:
one or several bacterial strain(s) able to convert primary substrates into succinate; and/or
one or several bacterial strain(s) able to convert succinate into propionate.

8. The composition for use according to any one of claims 4-7, wherein:
the strain able to convert primary substrates into formate is able to transform at least 20% of the total carbon transformed into the combined metabolites formate, lactate, succinate; acetate, propionate, butyrate and ethanol into formate when grown at least 48 hours in single culture on standard medium;
the strain able to convert primary substrates into acetate is able to transform at least 30% of the total carbon transformed into the combined metabolites formate, lactate, succinate; acetate, propionate, butyrate and ethanol into acetate when grown at least for at least 48 hours in single culture on standard medium;
the strain able to convert primary substrates into butyrate is able to transform at least 30% of the total carbon transformed into the combined metabolites formate, lactate, succinate; acetate, propionate, butyrate and ethanol into butyrate when grown for at least 48 hours in single culture on standard medium;
the strain able to convert primary substrates into lactate is able to transform at least 30% of the total carbon transformed into the combined metabolites formate, lactate, succinate; acetate, propionate, butyrate and ethanol into lactate when grown for at least 48 hours in single culture on standard medium;
the strain able to convert primary substrates into lactate is able to transform at least 30% of the total carbon transformed into the combined metabolites into propionate when grown for at least 48 hours in single culture on standard medium;
the strain able to convert primary substrates into lactate is able to transform at least 30% of the total carbon transformed into the combined metabolites formate, lactate, succinate; acetate, propionate, butyrate and ethanol into propionate when grown for at least 48 hours in single culture on standard medium;
the strain able to convert primary substrates into lactate is able to transform at least 30% of the total carbon transformed into the combined metabolites formate, lactate, succinate; acetate, propionate, butyrate and ethanol into succinate when grown for at least 48 hours in single culture on standard medium;
the strain able to convert formate into acetate is able to degrade at least 20% of formate and transform at least 20% of the total carbon transformed into the combined metabolites formate, lactate, succinate; acetate, propionate, butyrate and ethanol into acetate when grown for at least 48 hours in single culture on standard medium comprising or supplemented with formate;
the strain able to convert acetate into butyrate is able to degrade at least 20% of acetate and transform at least 20% of the total carbon transformed into the combined metabolites formate, lactate, succinate; acetate, propionate, butyrate and ethanol into butyrate when grown for at least 48 hours in single culture on standard medium comprising or supplemented with acetate;
the strain able to convert lactate into butyrate is able to degrade at least 20% of lactate and transform at least 20% of the total carbon transformed into the combined metabolites formate, lactate, succinate; acetate, propionate, butyrate and ethanol into butyrate when grown for at least 48 hours in single culture on standard medium comprising or supplemented with lactate;
the strain able to convert lactate into propionate is able to degrade at least 20% of lactate and transform at least 20% of the total carbon transformed into the combined metabolites formate, lactate, succinate; acetate, propionate, butyrate and ethanol into propionate when grown for at least 48 hours in single culture on standard medium comprising or supplemented with lactate; and/or
the strain able to convert succinate into propionate is able to degrade at least 20% of formate and transform at least 20% of the total carbon transformed into the combined metabolites formate, lactate, succinate;
acetate, propionate, butyrate and ethanol into propionate when grown for at least 48 hours in single culture on standard medium comprising or supplemented with succinate.

9. The composition for use according to any one of claims 1-8, wherein the composition does not comprise one or more bacterial strain(s) selected from the species Anaerotignum nov. sp., Clostridium_E sporosphaeroides, Coprococcus_A catus, Frisingococcus sp., Veillonella atypica, Veillonella parvula subsp. Parvula and Veillonella ratti; preferably selected from the genera Anaerotignum, Clostridium_E, Coprococcus_A, Frisingococcus, and Veillonella; even more preferably wherein the composition does not comprise a bacterial strain able to convert lactate into propionate.

10. The composition for use according to any one of claims 3-9,
wherein the one or several bacterial strain(s) able to convert primary substrates into formate is/are selected from the genera Anaerobutyricum, Bacteroides, Blautia, Collinsella, Coprococcus, Dorea, Erysipelatoclostridium, Escherichia, Eubacterium, Faecalibacterium, Lachnospira, Longicatena, Ruminococcus, and Sellimonas;
wherein the one or several bacterial strain(s) being able to convert primary substrates into acetate is/are selected from the genera Acidaminococcus, Acutalibacter, Bifidobacterium, Blautia, Clostridium, Clostridium_E, Clostridium_Q, Collinsella, Copromonas, Desulfovibrio, Dorea, Enterocloster, Escherichia, Eubacterium, Hungatella, Hungatella_A, Oliverbapstia, Peptoniphilus, Peptostreptococcus, Phocaeicola, Rhiziobiaceae genus, Ruminococcus, Sellimonas and Veillonella;
wherein the one or several bacterial strain(s) being able to convert primary substrates into lactate is/are selected from the genera Agathobacter, Bacteroides, Bariatricus, Bifidobacterium, Collinsella, Enterococcus, Lacticaseibacillus/Lactobacillus, Longicatena, Merdisoma, Peptostreptococcus, Roseburia, Streptococcus and Sutterella;
wherein the one or several bacterial strain(s) being able to convert primary substrates into succinate is/are selected from the genera Acutalibacter novel genus, Bacteroides, Oliverbapstia, Parabacteroides, Phocaeicola and Prevotella;
wherein the one or several bacterial strain(s) being able to convert formate into acetate is/are selected from the genera Blautia, Eubacterium; Terrisporobacter and Intestinibacter; and/or
wherein the one or several bacterial strain(s) being able to convert succinate into propionate is/are selected from the genera Dialister, Flavonifractor, Phascolarctobacterium Phascolarctobacterium_A and Veillonella.

11. The composition for use according to any one of claims 3-9,
wherein the one or several bacterial strain(s) able to convert primary substrates into formate is/are selected from the genera Blautia, Coprococcus, Dorea, Erysipelatoclostridium, Faecalibacterium, Lachnospira and Ruminococcus;
wherein the one or several bacterial strain(s) being able to convert primary substrates into acetate is/are selected from the genera Bifidobacterium, Blautia, Clostridium_Q, Clostridium_E, Desulfovibrio, Dorea, Eubacterium, Oliverpabstia and Rhiziobiaceae genus;
wherein the one or several bacterial strain(s) being able to convert primary substrates into lactate is/are selected from the genera Agathobacter, Bacteroides, Bariatricus, Collinsella, Enterococcus, Lacticaseibacillus/Lactobacillus, Peptostreptococcus, Streptococcus, and Sutterella;
wherein the one or several bacterial strain(s) being able to convert primary substrates into succinate is/are selected from the genera Acutalibacter nov genus, Bacteroides, Parabacteroides, Phocaeicola and Prevotella;
wherein the one or several bacterial strain(s) being able to convert formate into acetate is/are selected from the genera Blautia, Eubacterium, Terrisporobacter and Intestinibacter; and/or
wherein the one or several bacterial strain(s) being able to convert succinate into propionate is/are selected from the genera Dialister, Flavonifractor, Phascolarctobacterium and Phascolarctobacterium_A.

12. The composition for use according to any one of claims 3-9,
wherein the one or several bacterial strain(s) able to convert primary substrates into formate is from the genus Ruminococcus;
wherein the one or several bacterial strain(s) being able to convert primary substrates into acetate is from the genus Bifidobacterium;
wherein the one or several bacterial strain(s) being able to convert primary substrates into lactate is from the genus Lactobacillus;
wherein the one or several bacterial strain(s) being able to convert primary substrates into succinate is from the genus Bacteroides or Prevotella;
wherein the one or several bacterial strain(s) being able to convert formate into acetate is from the genus Blautia or Terrisporobacter, preferably from the genus Blautia and/or
wherein the one or several bacterial strain(s) being able to convert succinate into propionate is from the genus Phascolarctobacterium.

13. The composition for use according to any one of claims 3-9,
wherein the one or several bacterial strain(s) able to convert primary substrates into formate is/are selected from the species Bacteroides clarus, Blautia hydrogenotrophica, Collinsella aerofaciens, Coprococcus eutactus, Dorea formicigenerans, Dorea longicatena, Erysipelatoclostridium ramosum, Eubacterium ramulus, Faecalibacterium prausnitzii, Lachnospira eligens, Longicatena sp., Ruminococcus bromii and Sellimonas intestinalis;
wherein the one or several bacterial strain(s) being able to convert primary substrates into acetate is/are selected from the species Acidaminococcus intestini, Acutalibacter nov. genus having a 16SRNA sequence as set forth in SEQ ID NO: 9 and any variant thereof having at least 97%, 98% or 99% sequence identity thereto, Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium perfringens, Clostridium_E sporosphaeroides, Clostridium_Q symbiosum, Collinsella aerofaciens, Copromonas sp, Desulfovibrio piger, Dorea formicigenerans, Dorea scindens, Dorea sp. 900066555, Enterocloster sp., Escherichia coli, Eubacterium callanderi, Eubacterium limosum, Hungatella effluvii, Hungatella_A sp., Oliverpabstia sp., Peptoniphilus vaginalis, Peptostreptococcus anaerobius, Phocaeicola plebeius, Rhizobiaceae nov. genus, Ruminoccocus bromii, Sellimonas intestinalis and Veillonella ratti;
wherein the one or several bacterial strain(s) being able to convert primary substrates into lactate is/are selected from the species Agathobacter/Roseburia faecis, Bacteroides clarus, Bacteroides faecis, Bacteroides uniformis, Bariatricus comes, Bifidobacterium adolescentis, Collinsella aerofaciens, Enterococcus faecalis, Enterococcus faecium, Enterococcus gallinarum, Lacticaseibacillus rhamnosus/Lactobacillus rhamnosus, Longicatena sp., Merdisoma Aerosacheriphilus, Peptostreptococcus stomatis, Roseburia hominis, Streptococcus anginosus and Sutterella wadsworthensis;
wherein the one or several bacterial strain(s) being able to convert primary substrates into succinate is/are selected from the species Acutalibacter nov. genus having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto), Bacteroides faecis, Bacteroides fragilis, Bacteroides thetaiotaomicron, Bacteroides xylanisolvens, Oliverpabstia sp., Bacteroides faecis, Parabacteroides distasonis, Phocaeicola dorei, Phocaeicola plebeius, Phocaeicola vulgatus and Prevotella copri;
wherein the one or several bacterial strain(s) being able to convert formate into acetate is/are selected from the species Blautia hydrogenotrophica, Blautia_A luti, Blautia_A wexlerae in particular having a 16SRNA sequence as set forth in SEQ ID NO: 17 or a variant of at least 90%, 95%, or 99% identity thereto, Terrisporobacter othiniensis, Terrisporobacter glycolicus, Terrisporobacter mayombei, Intestinibacter bartlettii, Eubacterium callanderi and Eubacterium limosum; and/or
wherein the one or several bacterial strain(s) being able to convert succinate into propionate is/are selected from the species Dialister hominis, Dialister invisus, Flavonifractor plautii, Phascolarctobacterium faecium, Phascolarctobacterium_A succinatutens, Veillonella parvula subsp. Parvula and Veillonella ratti.

14. The composition for use according to any one of claims 3-9,
wherein the one or several bacterial strain(s) able to convert primary substrates into formate is/are selected from the species Blautia hydrogenotrophica, Coprococcus eutactus, Dorea formicigenerans, Dorea longicatena, Erysipelatoclostridium ramosum, Faecalibacterium prausnitzii, Lachnospira eligens and Ruminococcus bromii;
wherein the one or several bacterial strain(s) being able to convert primary substrates into acetate is/are selected from the species Bifidobacterium adolescentis, Bifidobacterium catenulatum, Bifidobacterium longum, Bifidobacterium pseudocatenulatum, Bifidobacterium ruminantium, Blautia hydrogenotrophica, Clostridium_Q symbiosum, Clostridium_E sporosphaeroides, Desulfovibrio piger, Dorea formicigenerans, Dorea longicatena, Dorea scindens, Dorea sp., Eubacterium callanderi, Eubacterium limosum, Oliverpabstia sp. And Rhizobiaceae nov. genus;
wherein the one or several bacterial strain(s) being able to convert primary substrates into lactate is/are selected from the species Agathobacter faecis, Bacteroides clarus, Bacteroides uniformis, Bariatricus comes, Collinsella aerofaciens, Enterococcus faecalis, Enterococcus faecium, Enterococcus gallinarum, Lacticaseibacillus rhamnosus, Peptostreptococcus stomatis, Streptococcus anginosus and Sutterella wadsworthensis;
wherein the one or several bacterial strain(s) being able to convert primary substrates into succinate is/are selected from the species Acutalibacter nov genus, Bacteroides faecis, Bacteroides fragilis, Bacteroides thetaiotaomicron, Bacteroides xylanisolvens, Parabacteroides distasonis, Phocaeicola dorei, Phocaeicola plebeius, Phocaeicola vulgatus and Prevotella copri;
wherein the one or several bacterial strain(s) being able to convert formate into acetate is/are selected from the species Blautia hydrogenotrophica, Blautia_A luti, Blautia_A wexlerae in particular having a 16SRNA sequence as set forth in SEQ ID NO: 17 or a variant of at least 90%, 95%, or 99% identity thereto, Terrisporobacter othiniensis, Terrisporobacter glycolicus, Terrisporobacter mayombei, Intestinibacter bartlettii, Eubacterium callanderi and Eubacterium limosum; and/or
wherein the one or several bacterial strain(s) being able to convert succinate into propionate is/are selected from the species Dialister hominis, Dialister invisus, Flavonifractor plautii, Phascolarctobacterium faecium and Phascolarctobacterium_A succinatutens.

15. The composition for use according to any one of claims 3-9,
wherein the one or several bacterial strain(s) able to convert primary substrates into formate is/are selected from the species Dorea formicigenerans, Dorea longicatena, Faecalibacterium prausnitzii and Ruminococcus bromii;
wherein the one or several bacterial strain(s) being able to convert primary substrates into acetate is/are selected from the species Bifidobacterium adolescentis, Bifidobacterium catenulatum, Bifidobacterium longum, Bifidobacterium pseudocatenulatum, Bifidobacterium ruminantium, Blautia hydrogenotrophica, Desulfovibrio piger, Dorea longicatena, Eubacterium callanderi, Eubacterium limosum and Rhizobiaceae nov. genus,
wherein the one or several bacterial strain(s) being able to convert primary substrates into lactate is/are selected from the species Bacteroides uniformis, Bariatricus comes, Collinsella aerofaciens, Enterococcus faecalis, Enterococcus faecium, Enterococcus gallinarum, Lacticaseibacillus rhamnosus, Streptococcus anginosus and Sutterella wadsworthensis;
wherein the one or several bacterial strain(s) being able to convert primary substrates into succinate is/are selected from the species Acutalibacter nov. genus, in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto, Prevotella copri and Bacteroides xylanisolvens;
wherein the one or several bacterial strain(s) being able to convert formate into acetate is/are selected from the species Blautia hydrogenotrophica, Blautia_A luti, Blautia_A wexlerae in particular having a 16SRNA sequence as set forth in SEQ ID NO: 17 or a variant of at least 90%, 95%, or 99% identity thereto, Terrisporobacter othiniensis, Terrisporobacter glycolicus and Terrisporobacter mayombei; and/or
wherein the one or several bacterial strain(s) being able to convert succinate into propionate is/are selected from the species Dialister hominis, Dialister invisus, Flavonifractor plautii, Phascolarctobacterium faecium and Phascolarctobacterium_A succinatutens.

16. The composition for use according to any one of claims 3-9,
wherein the one or several bacterial strain(s) able to convert primary substrates into formate is from the specie Ruminococcus bromii;
wherein the one or several bacterial strain(s) being able to convert primary substrates into acetate is/are selected from the species Bifidobacterium adolescentis, Bifidobacterium catenulatum, Bifidobacterium longum, Bifidobacterium pseudocatenulatum and Bifidobacterium ruminantium
wherein the one or several bacterial strain(s) being able to convert primary substrates into lactate is from the species Lactobacillus rhamnosus;
wherein the one or several bacterial strain(s) being able to convert primary substrates into succinate is/are selected from the species Acutalibacter nov. genus, Bacteroides faecis, Bacteroides fragilis, Bacteroides thetaiotaomicron, Bacteroides xylanisolvens, and Prevotella copri;
wherein the one or several bacterial strain(s) being able to convert formate into acetate is/are selected from the species Blautia hydrogenotrophica, Blautia_A luti, Blautia_A wexlerae, Terrisporobacter othiniensis, Terrisporobacter glycolicus, Terrisporobacter mayombei, and/or
wherein the one or several bacterial strain(s) being able to convert succinate into propionate is from the species Phascolarctobacterium faecium.

17. The composition for use according to any one of claims 3-9,
wherein the one or several bacterial strain(s) able to convert primary substrates into formate is Ruminococcus bromii;
wherein the one or several bacterial strain(s) being able to convert primary substrates into acetate Bifidobacterium adolescentis,
wherein the one or several bacterial strain(s) being able to convert primary substrates into lactate is Lactobacillus rhamnosus;
wherein the one or several bacterial strain(s) being able to convert primary substrates into succinate is/are selected from the species Acutalibacter nov. genus, in particular having a DNA genome sequence as set forth in SEQ ID NO: 1 or a variant of at least 90%, 95%, or 99% identity thereto and/or having a 16RNA sequence as set forth in SEQ ID NO: 9 or a variant having at least 95%, preferably at least 97%, more preferably 99%, 99.5%, 99.7% or 99.9% sequence identity thereto, Prevotella copri and Bacteroides xylanisolvens;
wherein the one or several bacterial strain(s) being able to convert formate into acetate is Blautia hydrogenotrophica; and/or
wherein the one or several bacterial strain(s) being able to convert succinate into propionate is Phascolarctobacterium faecium.

18. The composition for use according to any one of the preceding claims, wherein the bacteria consortium comprises or essentially consists of Agathobacter rectalis; Anaerostipes caccae and/or Anaerobutyricum hallii; Ruminococcus bromii; Bifidobacterium adolescentis; Lactobacillus rhamnosus; Phascolarctobacterium faecium and:
a bacterium selected from the group consisting of Bacteroides xylanisolvens, Prevotella copri and a bacterium Acutalibacter nov. genus having a DNA genome sequence having 90%, 95% or 99% sequence identity with SEQ ID NO: 1; and
a bacterium selected from the group consisting of Blautia hydrogenotrophica, Blautia_A luti, Blautia_A wexlerae, Terrisporobacter othiniensis, Terrisporobacter glycolicus, Terrisporobacter mayombei, Intestinibacter bartlettii, Eubacterium callanderi and Eubacterium limosum, preferably from the group consisting of Terrisporobacter glycolicus, Terrisporobacter othiniensis, Blautia_A luti and Blautia_A wexlerae in particular having a 16SRNA sequence as set forth in SEQ ID NO: 17 or a variant of at least 90%, 95%, or 99% identity thereto.

19. The composition for use according to any one of the preceding claims, wherein the bacteria consortium comprises or essentially consists of:
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and Bacteroides xylanisolvens;
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and Prevotella copri; or
Agathobacter rectalis; Anaerostipes Caccae; Ruminococcus bromii; Bifidobacterium adolescentis; Lactobacillus rhamnosus; Blautia hydrogenotrophica; Phascolarctobacterium faecium and a bacterium having a DNA genome sequence of SEQ ID NO: 1,
and wherein Blautia hydrogenotrophica can be replaced by a bacterium selected from the group consisting of Blautia_A luti, Blautia_A wexlerae, Terrisporobacter othiniensis, Terrisporobacter glycolicus, Terrisporobacter mayombei, Intestinibacter bartlettii, Eubacterium callanderi and Eubacterium limosum, preferably from the group consisting of Blautia_A luti, Blautia_A wexlerae, Terrisporobacter othiniensis, Terrisporobacter glycolicus, Terrisporobacter mayombei, even more preferably from the group consisting of Terrisporobacter othiniensis, Blautia_A luti, Blautia_A wexlerae, in the bacteria consortium.

20. The composition for use according to any one of the preceding claims, wherein the bacteria consortium comprises or consists of:
Agathobacter rectalis; Anaerostipes caccae; Ruminococcus bromii; Bifidobacterium adolescentis; Lactobacillus rhamnosus; Blautia hydrogenotrophica; Phascolarctobacterium faecium and Bacteroides xylanisolvens;
Agathobacter rectalis; Anaerostipes caccae; Ruminococcus bromii; Bifidobacterium adolescentis; Lactobacillus rhamnosus; Blautia hydrogenotrophica; Phascolarctobacterium faecium and Prevotella copri;
Agathobacter rectalis; Anaerostipes caccae; Ruminococcus bromii; Bifidobacterium adolescentis; Lactobacillus rhamnosus; Blautia hydrogenotrophica; Phascolarctobacterium faecium and a bacterium having a DNA genome sequence of SEQ ID NO: 1;
Agathobacter rectalis; Anaerostipes caccae; Ruminococcus bromii; Bifidobacterium adolescentis; Lactobacillus rhamnosus; Terrisporobacter othiniensis, Phascolarctobacterium faecium and Bacteroides xylanisolvens; or
Agathobacter rectalis; Anaerostipes caccae; Ruminococcus bromii; Bifidobacterium adolescentis; Lactobacillus rhamnosus; Blautia_A wexlerae, Phascolarctobacterium faecium and Bacteroides xylanisolvens.

21. The composition for use according to any one of the preceding claims, wherein said composition comprises between 5 and 15 different bacterial strains, preferably between 6 and 10 different bacterial strains.

22. The composition for use according to any one of the preceding claims, wherein the composition further comprises:
propionate, acetate and/or butyrate;
a pharmaceutical excipient or carrier; and/or
a component selected from the group consisting of cryoprotecting media, glycerol, culture or dispersing media, in particular comprising peptone, yeast extract, monosaccharides, disaccharides, arabinogalactan, fructo-oligosaccharides, fibers, glycerol, soluble starch, resistant starch, xylan, minerals, co-factors, vitamins and reducing agents; aqueous gels; prebiotics and polymeric supports; and any combination thereof.

23. A composition as defined in any one of claims 1-22.

24. A bioreactor comprising the composition of claim 23.

25. A method for treating an intestinal dysbiosis or a disease or disorder caused by or related to an intestinal dysbiosis in a subject, wherein the method comprises administering a composition according to claim 23 to said subject.

26. Use of a composition according to claim 23 for the manufacture of a medicament for the treatment of an intestinal dysbiosis or of a disease or disorder caused by or related to an intestinal dysbiosis.
